SEQUENCE LISTING (79 sequences)

SEQ ID NO: 1 - DNA, 597 bp, synthetic construct
  source 1..597: mol_type = other DNA
ctgactgaatacaagcctactgtcaggttggctacaagagacgacgttcctagagccgtgagaactctggctgcagccttcgccgactaccccgccacgagacacaccgttgacccagatcggcatattgagagagtgactgaactgcaggagctgtttcttacaagagttggcctcgacataggcaaggtgtgggtggcggacgacggcgccgccgtggccgtctggaccactcccgaatcagttgaggctggcgccgtattcgctgagatcggcccgagaatggctgagctcagcgggagtaggctcgcggcacagcagcaaatggagggactgctggcaccacacaggcccaaagaacccgcctggttcctggcaaccgtcggtgtatctcccgatcatcaggggaaaggtctgggctctgccgtagtgctccctggcgtggaggcagctgagagagcaggagtacctgccttcttggagacctccgctccaaggaatcttcccttctatgaacggttgggcttcaccgtgacagccgacgtggaagtccccgaaggcccccgcacttggtgcatgacgaggaagcctggagcg

SEQ ID NO: 2 - DNA, 585 bp, Rhodococcus aetherivorans
  source 1..585: mol_type = genomic DNA
acagagatcagacctgctgaacccgccgatgtggatcgcgcaacaagaacactggctagagcctttgccgactatcctttcaccagacacaccgtggacgcccgggaccacctgcggagagtggaagagctgcagcggctgtacctgaccgagatcggactgcggtgcggcagagtgtgggtcgccgatgatgcctccgccgtggccgtgtggaccacacctgagagcaccggcatccccgaggccttcgagcggatcgccggcagagtggccgagctgagcggcgaccgggccgacgccgctgctgccgccgaggaagccctggcccctctgagacccgtgggacctgtgtggttcctggccacagtggccgtggaccccgacagacagggctggggactgggcggcgccgtcctggaaccaggactgagggaagccagacaagctggcgtgccagcctacctggaaaccagcagcgagagaaacgtggctttctacagaagactcggcttcgacgttgtgggatctgtgaccctgcctggcgacggccctagaacctgggccatggtgcggaaccacacccag

SEQ ID NO: 3 - DNA, 594 bp, Prauserella flavalba
  source 1..594: mol_type = genomic DNA
tccggcgtgatgaaacctctgatccgggaagccaccagcgccgacatcgaccccgccaccgagacactgagagatgcctttgccgactaccccttcaccagacacacaatcgccgccgatgaccacctgggcagactggccagaatgcagagactgttcctggctagaatcggcctgccacatggccgggtgtgggtcagcgacgacgccgccgccgtggccgtgtggaccacccctgcttctaccggcctggaaagagtgttcaccgagctggcccctgagctgggcgccatcgcaggcgatagagccgctattgccgctgccacagaggccgccctggcccctcacagacctaccacccctagctggttcctgggaaccgtgggagtgcggcccggccagcagggccgcggactgggaagggctgttatcgagcctggcctgcgggccgctgaggccgaaggcgtgccagcttttctggaaacatctctggaaagcaacgtggccctgtaccggagattcggcttcgacgtggtggccgagatcgagctccctcaccacggccctagaacatgggccatgagcaagaagccc

SEQ ID NO: 4 - protein, 195 aa, Rhodococcus aetherivorans
TEIRPAEPADVDRATRTLARAFADYPFTRHTVDARDHLRRVEELQRLYLTEIGLRCGRVWVADDASAVAVWTTPESTGIPEAFERIAGRVAELSGDRADAAAAAEEALAPLRPVGPVWFLATVAVDPDRQGWGLGGAVLEPGLREARQAGVPAYLETSSERNVAFYRRLGFDVVGSVTLPGDGPRTWAMVRNHTQ

SEQ ID NO: 5 - protein, 198 aa, Prauserella flavalba
SGVMKPLIREATSADIDPATETLRDAFADYPFTRHTIAADDHLGRLARMQRLFLARIGLPHGRVWVSDDAAAVAVWTTPASTGLERVFTELAPELGAIAGDRAAIAAATEAALAPHRPTTPSWFLGTVGVRPGQQGRGLGRAVIEPGLRAAEAEGVPAFLETSLESNVALYRRFGFDVVAEIELPHHGPRTWAMSKKP

SEQ ID NO: 6 - DNA, 594 bp, Streptomyces mutabilis
  source 1..594: mol_type = genomic DNA
ctggaccctctgcctcatgtgcggcctgccgcccaggacgacgtgcctgccgctgttagaacactggccagagcctttgccgattaccccttcaccagacacgtggtggccgctgatggccaccaggagagagtgcggagattccaggagctgttcctgacaagggtggccatggaccacggcagagcctgggtgaccggcgactgcagagccgtcgccgcctggaccacccctgagcgggaccccggcccagccttcgccgaagtgggacctctggtgggcgacctggctggcgatcgggccgctgccctggcatctgccgagcaggccatggcccctcacagacctaccgacccagtgtggttcctggccaccgtgggcgtggaccctgacgcccaaggcgccggcctgggcaccgccgtgctgagacccggcctggaagccgctgaacgggctagattccccgcttttctcgagacaagcgacgagggcaacgtgcgcttctacacccggctgggattcgaggtgacagccgaagtcaagctgcctgatgacggccccctgacctggtgtatgagacgggaacctggaaga

SEQ ID NO: 7 - DNA, 591 bp, Streptomyces uncialis
  source 1..591: mol_type = genomic DNA
acaacagatgacagagtgcggccagccaccgaggccgatgtgcccgctgccgtgcgcaccctggccagagcctttgccgactaccccttcacccggcacgtggtggccgccgacgaccacacagaaagagtgcggagattccaggagctgttcctgaccagagtgggcctcgcccacggcagagtctgggtggccgatgatggcctgggagtggcagcctggaccacacctgagcaggaccctgctcctggactggccgaggtgggacctctggtgaccgaactggctggcgacagagcccctgcttttatggccgctgaggaagccctggcccctcatagacccaccgagcctgtgtggttcctggctacagtggccgtggaccccggcatccagtctaagggcctgggcgccgctgttctgagaccaggaatcgaggccgccgacagagccggccaccccgccttcctggaaaccgccacagagcggaacgtgaggttctacgagagactgggcttcagagtcaccgccggcaccaccctgcctgacggcggacctagagtgtggtgcatgagacgggaacctgccagc

SEQ ID NO: 8 - DNA, 570 bp, Kutzneria albida
  source 1..570: mol_type = genomic DNA
gtcgacctgagactggctacactggaagatgtgcccagagcagttgagacactgtctgccgccttcgccgactacgcctggctgcgccataccgtggctagagatagacacgccgagagagtgtccgagctggaacggctgttcgtggaacacgtgggcctcagacacggcagagtgtgggtcggcgacgacggagatgccgtggccgtgtggacccaccctgatacagacgtggccgctgcttttggcgccatcgcccctagaatgcgggaactggccggagacagagccgagtacgccgagcgggccgctgccgccctggcccctcacagaccaaccgagcctgtgtggttcctgggcagcctgggagtgcggcccgaggcccagggcaagggcatcggcggcgctatcgtgcagcctggcctgagagccgccgaagaggccggcgtgccagcctttctggaaaccagcgaggaaagaaacgtgcggttctacagaaagctgggcttcgaggtgaccgccgaggtgacaatccccgacggcggacctaccacctggtgcatgaggcgg

SEQ ID NO: 9 - DNA, 597 bp, Streptomyces sp.
  source 1..597: mol_type = genomic DNA
ctccaccaccaggacacccccagcgtgcggcctatcaccgacgccgatgtgcccaccgccgtggaaaccctggccagagccttcgccgattacccctatacacggcatgtggtggccgccgacgaccacgagggcagaatcagaagattccaggagctgtgtctgaccagagtgggcatggtctgcggccgggtgtgggtcgccgacgccggcagagctgttgctgtgtgggccacacccgaccaggaccctagccctgcttttgccgaaatcggacctctgctgggctctctggccggagatagagccgccgcctttgaaagcgccgagcaggccgtggccccttacagaccacaagagcctgcctggttcctgaacaccgtgggcgtgacccctgaggcccagggccagggcctgggctccgccgtgctggtgccaggcatcgaggctgctgctagagccggctaccctgtgttcctggaaacaagcagcgagcggaacgtgaagttctacgagagactgggattcgaggtgacagccgaagtggtgctgcctgacaatggccctagaacctggtgcatgcggaaggaccccaga

SEQ ID NO: 10 - DNA, 588 bp, Gordoni alkanivorans
  source 1..588: mol_type = genomic DNA
accatcagacctctgatccggcccgctacacctgctgatgtggacgccgctgccgtgaccctgggacaggccttcgccgactaccccttcacaagacacaccgtggacagccacgaccacggcgacagagtgcggagcctgcaaagactgttcctggctgaaatcggcatgcggtgcggccgggtgtgggtgtctgatgatctggccgccgtggccgtctggatcacccctagctctagcggactggacgaggctttcggcgacatcgccagcagagtggtggacctgtacggcgatagggccgagattgccgccagagccgatgaggccaccgccggactgagaccagccgagcctgtgtggcatctggccacagtgggcgttgctcctcacagccagggcagaggcctgggcgccgccgtcctggaacccggcctggccgcagcccagctggaaggccacgtggcctatctggaaaccagcagccccgccaacgtgtccttctacgagcggctcggatttgaggtggccggcaaggtgtccctgcctgacgacggccctgaggtgtgggccatgacctgtggcaga

SEQ ID NO: 11 - DNA, 594 bp, Photorhabdus sp.
  source 1..594: mol_type = genomic DNA
aatatgatcgtgcgggaaagcaaggaaatcagcgagatccagctggtgagatgtgtgcagaccctgacaagagcttttgacggctacagcctgatgcggcacttcctggccgaagatgaccaccagcagagagtgaggcggtaccaggagacattcctgagaaaggtgggaatgaccgtgggccacgtgtgggccgccgacgatggcgctgctgtgtccatctggaccgcccctgacatcgaggacgccgaggcaaccttcgcccctctgtctattgaattcggaaaaatcgccggcaccagagaaaaggtgatgagagccagcgagagcatcatggccaaggaacgccccaacttcccctgctggttcctgggcgccgtcgccgttgaccccgactaccagggcaaaggcctgggcagagccgtgatcgagcctggcctggaaagagccgagtgcgagggctttccagtgttcctcgagacatctgatgataagaacgtgcggatctacgagagactgggattcgaggtgaccgccgcttatcaactgcctttcggcggacctatgacctacgccatgatcaagcggggcatc

SEQ ID NO: 12 - DNA, 624 bp, Streptomyces clavuligerus
  source 1..624: mol_type = genomic DNA
acaacccctcctagccaccccgccgccgccagcagcggcccagtgcggcctgccacagatgaggacgtgccagctgcagttagaaccctggccagagccttcgccgcttatccttacacccgccacgtgatcgctgccgatggccacgaggaacgggtgcggagactgcaggagctgttcctcaccagagtgggcatggcctacggcagagtgtgggtcggaggcgagggcagagccgtggccgtgtggaccacacctgagcgggacccctctccaggattcgccgaagtgggacctcaaatcgccgagctggccggcgacagagccgccgcctacgaggccgtggaaagagctgtggacccctacagacccaaggaacctgtgtggttcctgggcagcgtggccgtggaccctgccgcccagggacagggcctgggcagcgccgtcatcagacctggcctggctgccgctgatgccgctggttgtcctgcttttctggaaacagccaccgagaggaacgtgcggctgtacgagagactgggcttcaccgtgaccgccgacctgcctggcagcgacggcggccccagaatctggtgcatgagacgggaacccggcgccggcgga

SEQ ID NO: 13 - DNA, 606 bp, synthetic construct
  source 1..606: mol_type = other DNA
accgctctcgatacaggcgtgcggcctgccgaaccccaggacacccctcgggccgtcagaacactgggcagagccctggctggctaccccgccctgcggcacacagtggaccctgatggacgtgccgagagagtgacagccatccaggagctgttcttcacccgggtgggcctggaagctggacgggtgtgggtcgccgacggcggcgacgccgtggccgtgtggaccacccctcacaacggcaatgccggcgccgtgttcgccgagatcggccccagactggccgagctgtgtggcacaagagccgccgctcaggaggccctggacaccgccctggcccctcatagaccaacagagcctgtgtggttcctggcaaccgtgggcgtgaccccagaaagacagggcgccggactgggcggagccgtgctgagacccggcatcgaggccgccgaggctgaaggcgttaccgcctttctggaaaccagcgaccccagaaacctgcctttctaccaaagactgggctttgagatctctgccgacgtgacccctgccgatggtggacctagaacctggtgcctgaggcggcctgctgctggccacgcctaa

SEQ ID NO: 14 - DNA, 576 bp, synthetic construct
  source 1..576: mol_type = other DNA
attcgggaagccaaccccgctgatatcgaccctgccaccgagacactgtgcgccgcttttgccgactaccccttcacaagacacaccatcgcagccgacgaccacctggaccggctggccagaatgcagcggagattcctgagcagaatcggcctgcctcacggccgcgtgtgggtctctgatgacgccggcgccgttgccgtgtggaccacccctgcctccaccggcatcggcagggtgttcaccgagctggcccctgagctggccgccatcgctggcgacagagccgctatcgccgccgctacagaggccgccctggctcctcatagacccaccacacctagctggttcctgggcaccgtgggagtgcggcctgatagacagggcagaggcctgggacgggccgtgatcgagcccggcctgagagccgctgaagccgagggcgtgccagcctttctcgagacaagcctggaaggcaacgtgaccctgtaccggaagctgggtttcgaggtggtggccgaaatcgagctgccacaccacggacctagaacctgggccatgagcaaggaaccttaa

SEQ ID NO: 15 - DNA, 594 bp, synthetic construct
  source 1..594: mol_type = other DNA
acatctccagccacagctgtgcggctggcaacacgggccgatgtgcccagagccaccgccacactgaccagagccttcgctgattacccattcacccggcacaccgtggccgccgacgaccacctgcggcggatcgccgagttccaggagctgtttgtggaccgcatcggcctggcccacggcagagtgtgggtcggcgacgagggagccgctgttgctgtgtggacaacccctgagaccgagggcgccgacgccgtctttgccgagctggctcctagattcgccgaactggccggagatagagcccaggccttcgagcaggccgaagccgccctggaacctcacagacctcaaggccctgcctggttcctgggcagcgtgggcgtggaccccgctcaccagggcagaggcctgggaagagctgtgctggcccctggcatcgaggccgctgaaagagccggcctgcccgcctacctggaaaccagcgaggccagaaacgtggctttctaccagagactgggcttcgccgtgtccgccgaggtggaactccctggaggcggccccctgacctgggccatgaccagacatggctaa

SEQ ID NO: 16 - DNA, 588 bp, synthetic construct
  source 1..588: mol_type = other DNA
agcatcagacccgccaccgccgccgatatcgacgccgctgccgtgaccctgcgcgaggcttttaccgactaccccttcagcagacacaccgtggccgccgacgaccatgccgctagagtggaacgggtgcagcacctgttcctgagccggatcggcctcccacacggcagagtgtgggtgtccgatgacgtggccgccgtcgcagtgtggacaacccctaccaccacagacctgaccgaggtgttcgccgagctgggacctgagctggccgaagccgctggcgacagagccgaggccgccgctgctgccgaggccgccctggcccctctgcggcctacaggccctgcctggttcctgggagtggtgggcgtgcggcctgatgctcagggcaggggcctgggccgggccatcatcgagccaggcctgagagccgccgctgaagccggcgttgaggcctacctggaaacatctctggaaaccaacctggccttttacagaaagctgggcttcgaggtcacaggcgagctggaactgcctggaggcggacctagaacctgggccatgagagccgctccccccgtgaagtaa

SEQ ID NO: 17 - DNA, 729 bp, synthetic construct
  source 1..729: mol_type = other DNA
acaggaaaccacaacggctcccctgccccaggcacaaccaccacccccaaggccgaccccaccgccggcaccgccaccgcccctgaggctggccccgaggccagagctgttgtgcggccagccacagccgaggacgtgcccagagccgtcagaaccctgacccaggcctttgccaactacccctggaccagacacaccgtggacgccgctgatcacgcccaccggatggaaagatttcaggagatcttcctgacacgggtgggcctggctcacggcagagtgtgggtcgccgacgacggcgacgccgtggccgtgtggaccacccctgagacagtgaacgccgaagccgtgttcgccgagctggcccctgagttcgccgccctcgctggagatagactgacagcctacgaggaagccgaggccgccctgctgccccaccggcctacagaacctgcatggttcctgggcaccatcggtgtgacccctgatagacagggcagcggactgggcagagccgtgattagacctggaatcgccgctgccgaaagagccggcgtgcctgcttatctggaaaccagcgacgagggcaacgtgcggttctacgagcgcctgggcttccaaatcacagctaccctgcacctgcctggcaatggccctcggacatggagcatgctgagaccacctagccccaccgcccctaggcctatcaccatgtctgatcatccttaa

SEQ ID NO: 18 - DNA, 600 bp, synthetic construct
  source 1..600: mol_type = other DNA
ccacaacaccaggatgctcctgatgtgcggcctctgacagacgccgatgtgcccatcgccgtggacaccctgaccagagccttcgtgggctaccccttcaccagacatgtgatcgccgccgacaaccacgagacaaggatcagaagattccaggagctgtgtctgacccgcattggcatggtgtacggcagagtgtgggtcgccgacgccggccgggccgtggccgtctgggccacaccagaccaggaccccagccctgctttcgccgaaatcggccctctgctcggcgacctgacaggcgatagaaccgccgcttatgagagcgccgagcaggccgtggccccttacagacctcaggagcctgcctggttcctgtccaccgtgggagtgacccctggcgctcagggcagaggcctgggaacagctgttctgatccccggcatcgaggaagccgaacacgccgagtgccctacctttctggaaacctctagcgagagaaacgtgaccttctacgagcggctgggatttaaggtgaccgctgaagtgctgctgcctggtagcggccccagaacatggtgcatgcggcgggaccctagataa

SEQ ID NO: 19 - DNA, 591 bp, synthetic construct
  source 1..591: mol_type = other DNA
acccacatcagactggccacagctgacgacattgcccctgctgccgacaccctggccgaggccttcgacggctatgcctttaccagacacaccgtggccgctgatggccaccgcgaccggctgcggagattccagagactgttcctggaaagaatcggcctcccctacggcagagtgtgggtggccgatgaccacgccgccgtggccgtgtggaccacacctgccaccgccgctgctggagatgtgttcgccggcgtggctgcagagctgatcgacatcgccggcgacagagccagacagcacgccgacgccgaagccgtgatggccagacatagaccaacagagcctgtgtggttcctgggaaccatcggagtgcggcctgacagacagggcgccggcctgggcagggccgttatcgcccctggcctggctgaggccgcccgggaaggcgtccccgcttttctggaaacctccatccggcggaacgtgacatggtacgagagcctgggcttcagagtgaccgccgattacgacctgcctgatggcggacctcacacatggtctatgctgagaccccccagcgccgagtaa

SEQ ID NO: 20 - DNA, 600 bp, synthetic construct
  source 1..600: mol_type = other DNA
acacctagaatccgggaagccacacctgccgacatcgagcctgctgttgctaccctgagcgccgcctttgccgattaccccttcaccagacacaccctggctgctgatgaccacctgacacggctggccgacatgcagagactgttcatcacccacattggactcccccacggcagagtgtgggtgtctgataacgcccacgccgtggccgtctggaccacaccagaaagcacagccatcgccgaagtgttcaccgacttcgcccctcaactggcccatatcgccggcgatagagcagctatcagcgccagaaccgagtctgctctggcccctcacagacctaccacccccacctggtttctgggaacagtgggcgtgcaccctgagtctcagggccagggcctgggaaaagccgtgatcgagcccggcctgcgcgccgccgacgccaccggcacagaggccttcctggaaaccagcctggccagcaacgtgacactgtaccggaagctgggcttcgacatcgtggccgagatcgacctgcctgacgacggccctaagacctgggccatgcggagaaagcccgctcctaccccagcctaa

SEQ ID NO: 21 - DNA, 597 bp, synthetic construct
  source 1..597: mol_type = other DNA
ccagccacaacacctagcgtgcgccccacccggcacgacgatgtgcctgctggcgtgcgggtgctggctagagccttcgccgactaccccttcaccagacacgtggtcgccgctgatgatcaccccagaagagtgaggcggctgcaggagctgttcctggccagaatcgccctgccttacggcagatcctgggtcaccgacgacggcctggccgtggccgcctggaccacccctgagcgggacccagaacctgcctttgccgaaatcgcccctgtgatcgccgaactggccggatctagatgggccgcttatcaggccgccgaggaagccctggcaccacatagacctgcccaccctgtgtggttcctggctacagtgggcgttgaccctgacgctcagggccaaggaagaggcgccgctgtgctgagacccggcctggaagccgccgaggccgccggcctgcctgcttttctcgagacaagcgaccccggcaacgtgcggttctacgagagactgggcttcaccgtgaccgccgaggtgcccctgcctgatggcggacctctgacctggtgcatgctgagagcccctggcagataa

SEQ ID NO: 22 - DNA, 597 bp, synthetic construct
  source 1..597: mol_type = other DNA
tccgtgacaatccctccaaccagaagaacaacccatgatgacgtgcccgcctgcgtggaagttctgacccgggcctttgccgactaccccttcaccagacacgtggtggccgctgatgaccacgagagaagggtgcggagactgcaggagctgttcctgaccagagtggccctgagacacggacggagctgggtcaccgacgatagactggctgtggcagcctggaccacccctgaacaggaccccagccctgccttcgccgaaatcggcagcctgctgcctgagctggccggcgacagagccgctgcctacgaggccgccgaggaagccctggcccctcacagacctacccaccctgtgtggttcctggccaccgtgggcgtggcccctgaggctcagggcagaggccggggcgccgccgtgctgcggcctggcctggaagccgctgaagctacaggcttccccgctttcctcgagacatctgatgccagaaacgtgcggttctacgagcggctgggctttaccgtcaccgccgaggtgcctctgccagacggcggacctctgacatggggaatgacaagaagccccggccgctaa

SEQ ID NO: 23 - DNA, 600 bp, synthetic construct
  source 1..600: mol_type = other DNA
acaaccaacgcccctgtggtcagacctgctacacgggacgacctgccaagagccctgcggaccctgcagagagcctttgccgattacgccttcacccgccacaccatcgccgctgatggccatctggaccggctgcacagattcaacgagctgttcgtgacaagaatcggcctggaacacggcagagtgtgggtggccgacggcggcgccgctgttgctgtgtggaccacacctgagacagccgaggccggaagcgtgttcgccgaactgggacctctgtttgctgagatcgccggcgacagagccgaaatcttcgcccagaccgaggccgctctgggacctcaccggcccaccggccctgtgtggttcctgggatctgtgggagtggaccctgatagacagggcaggggcctcggcggagccgtgatcagacccggcctggaagctgccgatgccgccggcgtgcccgccttcctggaaaccagcgacgagagaaatgtgcggttctacgagcggctgggcttcgaggtgaccgccgagtgcgtgctgcctggcggcggacctagaacctggtccatgagcagaaagcctgtcagctaa

SEQ ID NO: 24 - DNA, 600 bp, synthetic construct
  source 1..600: mol_type = other DNA
acaaccagcacccctgccgtgcggcccgctacacgcgacgacctgcctagagccctgcggaccctgagaagggccttcagcgactaccccttcactcggcacaccatcgccgctgatggccacctggacagactgcacagattcaacgagctgttcctgaccagaatcggcctggaacacggcagagtgtgggtcgccgatggaggcgccgctgtggccgcctggaccacacctgaaaccgccgaggccggatctgttttcgccgagctgggacctctgtttgccgagatcgccggcgaccgggccgaaatcttcgcccagaccgaggccgctctgggacctcaccggcctacaggccctgtgtggttcctgggaagcgtgggcgtggaccctgatagacagggcagaggcctcggcggagccgtgatcagaccaggcctggaagctgccgacgccgctggcgtgcctgcttttctggaaacatctgatgagagaaacgtgcggttctacgagcatctgggcttcgaggtgaccgccgagtgcgtgctgcccggcggcggaccaagaacctggagcatgagcagaaagcctgtgtcctaa

SEQ ID NO: 25 - DNA, 600 bp, synthetic construct
  source 1..600: mol_type = other DNA
accatgagcacacctgccgtgcggcctgctacacacgacgacctgcctagagctctgagaaccctgcagagagccttcagcgactaccccttcaccagacacaccatcgccgctgatgaccacctggaccggctgcacaggttcaacgagctgttcgtgacaagaatcggcctggaacacggcagagtgtgggtggccgatggcggagccgccgtcgccgtctggaccacacctgagacagccgaagccggcagcgtgttcgccgagctgggacctctgtttgctgagatcgccggagatagagctgaaatcagcgcccagaccgaggccgctctgggaccacatcgccccaccggcccagtgtggttcctgggaagcgtgggcgtggaccccgacagacagggcagaggcctgggcggcgccgtgatcagacctggcctcgaagccgccgacgccgccggcgttcccgcttttctggaaacctctgatgagcggaacgtgcggttctacgagcacctgggcttcgaggtgaccgccgagtgcgtgctgcctggcggcggccctcggacctggtccatgtctagaaagcctggaccttaa

SEQ ID NO: 26 - DNA, 600 bp, synthetic construct
  source 1..600: mol_type = other DNA
accaccaatacccctgtggtgcggcctgccaccagagatgatctgccaagagccctgagaaccctgcaaagagccttcgccgactacgccttcacacgccacaccatcgccgctgacggccacctggaccggctgcacagattcaacgagctgttcgtgaccagaatcggcctggaacatggaagagtgtgggtcgccgacgacggcgacgccgtggccgtttggaccacacctgagacagccgctgccggcaacgtgttcgccgaggtgggacctctgtttgccgagatcgccggagatagggctgaaatcagcgcccaggccgaagctaccatgggacctcaccggcctacagagcctgtgtggttcctgggctccgtgggcgtggaccccgacagacagggcagaggcctgggaggcgccgtgatcagacctggactcgaagccgccgacgccgctggcgtccccgcctttctggaaacatctgacgagcggaacgtgcggttctacgagagactgggcttccaggtgaccgccgattacgtgctgcccggcggcggacctagaacatgggccatgagcagaaagcctggcgcttaa

SEQ ID NO: 27 - DNA, 600 bp, synthetic construct
  source 1..600: mol_type = other DNA
agccaacatcagaacgcccctagcgtgcggccaatcaccgacgccgacgtgcccgctgcagtggacaccctggccagagccttcgccgactacccttacaccagacacgtgatcgccgccgacggccacgaggaacggattagaagataccagcagctgtgcctgacccggatcggcatggtgtacggcagagtgtgggtcgccgatgagggcagagccgtcgccgtgtgggccgttcctggtcaggaccctagccctgctttcgctgaactgggacctatcctgggcgagctgtctggcgacagagccgccgtgtccgccacagccgatgccgctatggccccttatagacccaaggaacctggctggttcctggaaacagtggctacagccccagaggctcagggcaaaggcctgggatctgccgtgctgatccccggcatccaggaggccgagagagccggatgtcctgccttcctggaaaccagcagcgaggctaatgtgcggttctacgagaggctcggatttaaggtgaccgccgatgtgcagctgcctggcaacggccccagaacctggtgcatgcgccgggacccccactaa

SEQ ID NO: 28 - DNA, 603 bp, synthetic construct
  source 1..603: mol_type = other DNA
cccacctcctgcagccctagcgtgcggcctgccacacgggccgacctgcctagaatcctgagaaccctggaaggcgcttttaccgactacccactgacaagacacaccctggccgcagatggccacgccgacagactgcggagattcaacgagctgttcgtgacccgggtgggcctggaccacggcagagtgtgggtggccgatggcggcgccgccgttgccgtctggacaaccccagaaaccgccgaggccggcgacgtgttcggcgagctgggacctcggttcgccgagatcgccggagatcgcgccgaaatcagcgcccagaccgaggccgctatgggcgtgcacagacctacagagcctgtgtggttcctgggcaccgtgggtgtggaccccggaagacagggccagggcctgggcgccgccgtgatcagacccggactcgaagccacaggcgctgctggcgtccctgcttttctggaaacctctgacgccagaaacgtgaggttctacgagcggctgggcttcgaggtgaccgctgattaccccctgcccggcggcggacctagaacatgggccatgacccataagcctggcgcctaa

SEQ ID NO: 29 - DNA, 597 bp, synthetic construct
  source 1..597: mol_type = other DNA
accgagcaggcccctgctgtgcgggcagccacacgggaagatctgccaagagccgtgcggacactgggcagagcttttctgcactaccccctgaccaggcatacaatcgccgccgatgaccacgccgccagactggaaagattcaaccacctgtttgtcagcagaatcggactcgagcacggcagagtgtgggtgtctgatgattgcgccgccgtggccgcttggaccacccctgccaccgacgccgccgccgttttcggcgagatcggccctgagctggaaagactggccggcgacagagccccattcgccgctcgggccgaggaaaccatgcggccccacagacctactgtgcctacatggttcctggctacaatcggcgtggaccctggcagacagggacaaggcctgggaagagccgtcgtgctgcctggagtggaagccgctgagcgcgctggcgtgcccgccttcctggaaaccagcgacgagcggaacgtgcggttctaccagggcctgggcttcgaggtgaccgccgactacgccctgcctgacggcggccccagaacctgggccatgaccagagagcctggcgcctaa

SEQ ID NO: 30 - DNA, 597 bp, synthetic construct
  source 1..597: mol_type = other DNA
agcgtgacaacccctccagccagaccaacaacacatgatgatgtgcctgcatgcgtggaagtgctgaccagagccttcgccgattaccccttcacccggcacgtggtcgccgccgacgaccacaagtggcgggtgcggagactgcaggagctgttcctcgccagagtggccctgagatacggcaggtcttgggtcaccgacgacagactggccgttgccgcctggaccacccctgagcaggacctgtcccctgcctttgccgagatcggcagcctgctgcctgaactggccggagatagagccgctgcttatgaggccgccgaggaagctctggcccctcacagacctacacaccccgtgtggttcctggctacagtgggcgtggctcctgaggctcagggccggggccgcggcgccgctgtgctgcggcctggactggaagccgctgaggccgccggcttccccgccttcctggaaaccagcgacgccagaaacgtgcggttctacgagagactgggatttaccgtgaccgccgaggtgcccctgcccgacggcggccctctgacttggggcatgaccagaagccctggcagataa

SEQ ID NO: 31 - DNA, 579 bp, synthetic construct
  source 1..579: mol_type = other DNA
attatcagacccgctacagccgccgacgtggacgccgccgtgaccaccctgtctatggccttcgccgattaccccttcacccggcacacaatcgccgccgacgaccacgccggcagactggctagaagccagagactgtttctgaccagaatcggcctgccccacggcagggtgtgggtgtccgaccatgccgaggccgtcgccgcttggacaacccctgatgccgcagacctgggcagagtgttcgccgatgtggccccagagctggccgagctggccggagatagagccgaaatcgccgctgagagcgaggccgctctggctccttttagaccaacaggccctgcctggttcctgggtacagtgggagtgcggcctggaaaccagggccggggcctgggccgcgccgtcatccaacctggcctcgacgccgctgaagccgacggcgtgcaggcctacctggaaaccagcaccgagcggaacgtggaactgtaccggaagctgggcttcgaggttgtgggcgaggtggaactgcctagaggcggacctaagacctgggccatgcggagaggctgctaa

SEQ ID NO: 32 - DNA, 615 bp, synthetic construct
  source 1..615: mol_type = other DNA
cggaccgagcagtctagccaacctgctccacctaccgtgaggtccgccacacccgctgatatccccagagccaccagaacactgggcagagcctttgccgactacgcctggacccggcacaccatcgacgccagagaccacgaacagagagtgcggggaatgcaggagctgttcctgacccacatcggcctcccccacggccgcgtgtggatcgccgacgagggcgccgctgtggccgtgtggacaacacctgccaccgatgccggccctgccttcgctgaactggcccctagattcgccgatctggccggcgacagagccgccgcctacgccgctgccgacgccgccctggccccacatagacccgtcgagcctgtgtggttcctgggtacagtgggcgtggaccccgacagccagggcagaggcctgggcggcgccgtgatccggcctggactggctgccgccgatagagcaggcgttcctgcttttctggaaaccagcgagaagcggaacgtgggattctacgagcggctgggcttcagagtgaccgccacagtggacctgcctgacggcggacctacaacctgggccatgctgagagatcctggcgcttaa

SEQ ID NO: 33 - DNA, 564 bp, synthetic construct
  source 1..564: mol_type = other DNA
accactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggttgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccc

SEQ ID NO: 34 - DNA, 561 bp, synthetic construct
  source 1..561: mol_type = other DNA
acaaccgtggacgacatggcctacgagttcagaaccgccagacctgaggataccgaggccattgaagccctggatggcagcttcaccacccacaccatcttccaagtggccgtgacagaaaccggcttcgccctccaggagatccctgtggacccccccatccataaggtgttccccgctgaaaacacagctgatgcccctgttgccgagggagatccttctagcagaacctttgtggccgtgggaaccgacggcagcctggctggattcgctacagtgtcctacgccagctggaatcggagactggccatcgaggacatcgaagtggtccccgcccacagaggccgcggcgtgggcagagccctgatcggccacgccgtgaccttcgccagagagagcggcgccggccacatctggctggaagtgacaaacatcaacacccctgctatccacgcctatcagcggatgggctttaccttctgcggcctggacacaacactgtacgacggcaccccatctagcggcgagcaggccctgtatatgagcatgccttgtccttaa

SEQ ID NO: 35 - DNA, 555 bp, synthetic construct
  source 1..555: mol_type = other DNA
accaccgccgatgagacaacctacgagttccgggccgctagacgggaagatttcgaggccatcgacgccctggacggcagcttcaccaccagcaccgtgttcagagtggacgtgacaggcgacggatttgccctgagagaggtccccgtggaccctccactgaccaaggtgttccccgaggacgagtctgaaggcgccgacggcgccgacagcggctctagaaccttcgtggctgttggagctgacggcgagctggccggattcgccgccgtgtcctacagcccttggaaccggcggctgacagtggaagatatcgaggtggcccctggccacagaaatagaggcgtgggccacgccctcatgggccacgccgtggacttcgccagagaatgtggcgctggacatgtgtggctggaagtgaccaacgtcaacgcccctgctatccacgcatatagaaggatgggctttgccttctgcggcctggatacagccctgtaccagggcacagagagcgagggcgaacaggccatctacatgagcatgccttgcccctaa

SEQ ID NO: 36 - DNA, 573 bp, synthetic construct
  source 1..573: mol_type = other DNA
accaccatcggagccatggactacgagttcagaacagccagacctcccgatacccctgctatggaagccctggatggatcttttaccacaagaaccatcttccacgtggctgctacagaggatggcttcgccctccaggagatccccgtggaccctccactgcacaaggccttccccgctggcgacagcgacgccgatgccgacgacggactgaccacagaagaggaccccaatagcagaaccttcgtggccgtgggacctgatggttgtctggccggatttgccgctgtctcctacgccccttggaaccggcggctggccattgaggacatcgaggtggcccctgcacatagaagccagggcctgggcagagccctgatggcccacgccgccgacttcgccagggaaagaggcgccggccacatctggctggaagtgaccaacatcaacgccccagctatccacgcctaccggagaatgggcttcacattctgcggcctggacaccacactgtacgacggcacccctagcagcggcgagagagccctgtatatgtctatgccttgcccttaa

SEQ ID NO: 37 - DNA, 549 bp, synthetic construct
  source 1..549: mol_type = other DNA
acaaccgttggcgacaccgcttaccgctaccggatcgccgctgctggagatatcgaggccatcagagccctggatgatagcttcaccacacacaccgtgttcagagtgaccgtgacagaggaagccttcgccctgcgggaaatccccgtggaaccccccctgaccaaggtgttccctaagaacgagcctgacgacgaggacgacgccgacagcagagcctttgtggcccacggcgccgctggcgacctggccggatttgccgccgtgtcctacagcggctggaatagaaggctgacaatcgagaacattgtggtcgcccctccacatagaggaagaggcgtgggcagagccctgatcgagctggccaagaaattcgctagagagagagatgccggccacctgtggctggaagtgaccaacatcaacgcccctgcaatccacgcctatcggagaatgggcttcgccttctgcggcctggacaccaccctgtacgagggcacacctagcaagggcgaacaggccctctacatgtctatgccttgtctgtaa

SEQ ID NO: 38 - DNA, 555 bp, synthetic construct
  source 1..555: mol_type = other DNA
acaacagccggagatacaccttaccgctacagagtggccgctcctgaggacaccgaggccgtgagagccctggacgcctccttcaccaccgacaccgtctttcaggtgaccgttacagaggaaggcttcgccctgcgggaaatcagaatggaactgcctctgacaaaggtgttccccgaggacgagcccgacgacgacgccgaggacgatgctgatagccggaccttcatcgcccatgatgccgccggcgacctggctggcttcgtgacagtggcttattctggctggaatagacggctgaccgtggaagatatcgccgtggtgccccagcacagaggcaggggagtgggaagagccctggtgggcctggccagaaagttcgctagagagagaggcgccggccacctgtggctggaagtgaccaacatcaacgcccctgccatccacgcctaccggagaatgggctttgccttctgcggcctggacaccaccctgtacgagggcacccctagcagaggcgagcaggccctgtatatgagcatgccatgtcactaa

SEQ ID NO: 39 - DNA, 567 bp, synthetic construct
  source 1..567: mol_type = other DNA
acaaccgtggacaccatgaactacgagttcagaaccgcccgacctgaggataccgaggccatcgacgccctggatggcagcttcaccaccagaacaatcttccacgtggccgtgacagaaggcggattcgtcctgcaggagatccccgttgatcctccaatccataaggtgttccctgctgaagataccgacgacggcaacagcccagccgctggcgaggaccccaattctagaaccttcgtggctatcggcgccgacggcggcctggccggctttgccgctgtgtcttacgccccttggaacggcagactgacaatcgaggacatcgaggtggcccccgctcaccggggacagggcgtgggcagagccctcgtgggccacgccgccgagttcgcccgggaaagaggagccagacacatctggctggaagtgaccaacatcaacgcccctgccattcacgcctacagaagaatgggcttcagcttttgtggcctggacatggccctgtatgacggaacacctagcagcggcgaacaggcactgtatatgagccggtcctgcctgtaa

SEQ ID NO: 40 - DNA, 654 bp, synthetic construct
  source 1..654: mol_type = other DNA
accaccgccgacgatacaccttacgaaatcagaatcgccgccagagaagatgccggagccctgaaggccctggacggctccttcacaacaaccaccgtgttccacgtggaaaccagcgagaacggcttcgccctgagagagtctctgattgagcctccactgacaaaggtgttccccgaggatgatcagggcgacagcgacggcgacgacgagagaggcagagtggaccagaatagcagaaccttcctggctctgggcgctgatggcagcctggctggatttgtgtccgtggcctatgccccttggaaccggagactgaccatcgaggacatcgaggtggctcctgaacaccggggccggggcgtgggaagagccctggttggacgggctgaaggcttcgctagagagagaggcgccggccacatctggctggaagtgaccaacgtcaacgtgccagccgtgcgggcctacagaaggatgggctttgtgctgtgcggcctggacacatctctgtacgagttcaccgccagcgccggcgagtacgccctgtatatgcgaaaaccttgtagaccccacagacctgccctcacccccagccccaccgagacacctctgaccgccgctcatagatctgccgaaagcagcacaagctaa

SEQ ID NO: 41 - DNA, 567 bp, synthetic construct
  source 1..567: mol_type = other DNA
acaacagttgacgatacaacctacgccctgagaaccgcccggcctgaggacgccgaagctattgaggccctggacggctcttttacaacaagcaccgtctttagagtggaaatcgccgagaatggcttcaccctgcgggaaacccctgtggacccccccctgacaaaggtgttcccagaagatgagtctgatggcgacgacgaggatggcggacctgaggaccaggacagccccaccttcctggctctgggcgccgacggcagcctggctggattcgtgtccgtgtcctacgccccatggaaccggagactgaccatcgaggacatcgaggtggcccctggccacagaggcagaggagtgggcaggatgctgatggccagagccgaggaattcgccagagagcggggcgctggccaggtgtggctggaagtgaccaacatcaacgcccctgctatccacgcctacagacgcatgggcttcagcctctgtggcctggataccagcctgtacgagttcaccagcagcgccggagaacacgccctgtatatgagcaagccttgcagctaa

SEQ ID NO: 42 - DNA, 588 bp, synthetic construct
  source 1..588: mol_type = other DNA
cctcctgctgatgataccacctacgagttcagaaccgccacccctgaggacaccacactggtggaagccctggacggcagcttcaccacagccacagtgttcagagtggaaatggccgagaacggctttaccctgagagagacacctgtggaccctccactgacaaaagtgttccccgaggatgagggcgacgaggaagatgacggcgctgaagaggacggcgtcaaggaagaaaaccccaccttcctggccgtggccccagacggaagcctcgccggcttcgtgtccgtggcttatgccagatggaaccggcggctgaccgtggaagacatcgaggttgctcccggccacagaggacggggcgtgggcagagccctgatgagcagagccgaggaattcgccagagagaggggcgccggacacatctggctggaagtgaccaacatcaacgcccctgccatccacgcctaccgcagaatgggattttctctgtgcggcctggacaccagcctgtacgagttcacagcctctgccggcgagtacgccctgtacctgagcaagccttgtagaggcgctaatagagattaa

SEQ ID NO: 43 - DNA, 564 bp, synthetic construct
  source 1..564: mol_type = other DNA
cctccagctgatgatacaacctacgagatcagaatcgccacacctgaggacaccggccccgtggaagctctgggcggcagcttcaccacagccaccgtgttcagagtggaaatggccgagaacggctttacactgcgggaaacccctgtggaccctccactgaccaaagtgttccccgaggatgaggatgacgacgaggccgaagaggacggcgccaaggaaggccatcctaccttcctggccgtggctcccgacggctctctggctggattcgtgtccgtggcctacgccagatggaatagaaggctgaccatcgaggacatcgaggtggcccctggccacagaggcagaggcgtgggccgcgctctgatgagcagagccgaggaattcgcccgggaaagaggagccggccacatctggctggaagttacaaacatcaacgcccctgctattcacgcctatagacggatgggatttgccctctgtggcctggacaccagcctgtacgagttcaccgccagcgccggtgagtacgccctgtacctgagcaagccctgcagataa

SEQ ID NO: 44 - DNA, 564 bp, synthetic construct
  source 1..564: mol_type = other DNA
ccacctgctgatgatacaacatacgagattagaatcgccacacctgaggacaccggccccgtcgaggctctgggcggcagcttcaccacagccaccgtgttcagagtggaaatggccgaaaacggatttaccctgcgggaaacccctgtggaccctcctctgaccaaggtgttccccgaggacgaggacgacgatgaggccgaggaagatggcgccaaggaaggccatcctacattcctggccgtggccccagacggcagcctggccggattcgtgtccgtggcttatgccagatggaatagacggctgaccatcgaggacatcgaggttgcacccggccacagaggaagaggcgtgggccgcgccctgatgagcagagccgaggaattcgctagagaacggggtgctggccacatctggctggaagtgaccaacatcaacgcccccgctatccacgcctaccggcggatgggctttgccctgtgcggcctggacaccagcctgtacgagttcaccgccagcgccggcgagtacgccctctacctgtctaaaccttgtagataa

SEQ ID NO: 45 - DNA, 564 bp, synthetic construct
  source 1..564: mol_type = other DNA
cctccagccgacgacacaacatacgagatcagaaccgccacacctgaggacaccgccctggtggaagccctggatggcagcttcaccaccgcaacagttttccaggtggaaaccgccgaaaacggctttaccctgcgggaaacccctgtggacccccccctgacaaaggtgttccccgaggatgaggaatacgacgaggccgaggaagatggcgccaacgagggcaaccctacattcctggccgtgaccccagatggcagcctggctggctttgtgtccgtggcctacgcccggtggaatagacggctgaccgtcgaggacatcgaggtggctcctggccacagaggaagaggcgtgggcagagccctgatgagcagagccgaggaattcgccagagagagaggcgctggacacatctggctggaagtgaccaacatcaacgcccctgctatccacgcctatagaaggatgggcttcgccctgtgcggcctggacacaaccctgtacgagttcaccgcttctgccggcgagtacgccctctacctgagcaagccctgtccttaa

SEQ ID NO: 46 - DNA, 558 bp, synthetic construct
  source 1..558: mol_type = other DNA
acaacaacccacgacacaacctacgccttcagagtggctagacctgaggacgtggaagccatcgccgccatcgacggcagcttcacaaccggcaccgtgtttcaggtggctgtggcccctgacggcttcaccctgcgggaagtcgctgttgacccccccctggtgaaggtgttcccagaggacgacggctctcacgacgccgagggagaggatggcgatagaaggacctacgtggccgtgggcgctggcggagccgtcgccggcttcaccgccgtgtcctacaccccttggaacggcagactgacaatcgaggatatcgaggtggcccctggccatagaggcagaggaatcggccggggactgatggaacgggccgctgatttcgcccgggaaagaggcgcaggccacctgtggctggaagtgaccaatgtgaacgcccctgccattcacgcctatctgagactgggctttacattctgcggcctggacaccgccctctacctgggaaccgagagcgagggcgagcaggccctgtatatgagcatgccctgtccttaa

SEQ ID NO: 47 - DNA, 585 bp, synthetic construct
  source 1..585: mol_type = other DNA
accactccacacggcccggccgacggaatcgtctaccgcctcgcccgccccgaggacgcgggcgccatcgaggccctggacagctccttcaccacccccaccgtcttcgaggtgaccgcctccggcgacggcttcggcttcctgctccgcgaggtccccgtcgacccgcccgtgcacaaggcgttcccgccggaggagcacgacgagcaggggttcgccggcgcccggggccccgacgtggacgcggacgcgcgcaccttcgtggccctcgacggcggcgagctgtgcgggttcgccgccgtcggctacgccgcgtggaaccggcggctgaccgtcgaagacatcgaggtcgcgccgggccaccggggccgcgggatcggcagcgccctgatggagcgtgccgccgagttcgcccgcgagcggggcgcggagcacctctggctggaggtcagctcggtcaacgcccccgccgtgcacgcctaccggcgcatgggattcaccttctgcggcctcgacaccgccctctacggcggcacgccctccgcgggcgaacgggcgctgttcatgagccgcccctgccgctaa

SEQ ID NO: 48 - DNA, 573 bp, synthetic construct
  source 1..573: mol_type = other DNA
accacagtggacgacaccacctacgagttcagaaccgccagacctgaagatgcagaagctgtggaagccctggacggatctttcaccaccgctacagtgttcagagtggaaatcgccgaaaacggctttaccctcagagagacacctgtggacagacccctgaccaaggtgttcccagaggatgagagcgacggcgacgacgacgaggatgacggcggcagcgaggaccctgattcccctacctttctggctctgggcgctgatggcacactggctggcttcgttagcgtgtcctacgccccttggaatagacggctgacaatcgaggacatcgaggtggccccaggccacagaggaagaggcgtgggcaggatgctgatggcccgggccgaggaattcgcccgcggaagaggcgccggccacgtgtggctggaagtgaccaacatcaacgcccctgccatccacgcctacagacggatgggattcagcctgtgtggcctggacaccagcctgtacgagttcacaagcagcgccggcgagtacgccctgtatatgtctaagccctgcccctaa

SEQ ID NO: 49 - DNA, 567 bp, synthetic construct
  source 1..567: mol_type = other DNA
accgcgaaccatggcacgacgtacgagttccgcaccgcacgccccgaggacaccggggccatcgaagccctcgacgggtccttcaccaccggcaccgtcttcgaggtggccgtcaccggcgaggggttctccctgcgcgaggtcccggtggaccccccgctggtcaaggtgttccccgaggacgacggcagcgacgaggaggacggcgcggagggcggggacggcgacagccgcacgttcgtggccgtctgcgccggaggcggcctcgccggcttcgccgccgtgtcctactcgccgtggaaccggcggctgaccatcgaggacatcgaggtcgcccccgaccaccggggccggggcatcggccgtacgctgatccggcacgccgtggacttcgcccgcgaacgcggcgccggacacctgtggttggaagtgaccaacgtcaacgcccccgccatccacgcctaccgccgcatgggcttcgccttctgcggcctggacaccgccctgtaccagggcaccgagtccgagggcgagcacgcgctctacatgagcatgccctgcccctaa

SEQ ID NO: 50 - DNA, 585 bp, synthetic construct
  source 1..585: mol_type = other DNA
acaaccgcccatggccctgccgacggcatcgtgtaccggctggccaggcctgaagatgccggcgccgtcgccgctctggacagcagcttcacaacaagaaccgttttcgaggtggcagtgagcggcgacggcagcggatttctgctgcgcgaggtgcccgtggaccccccagtgcggagagccttccctcctgaggaacacgacgagcagggcatcgccggcccaagaggagctgatgtggacgccgataccagaaccttcgtggctctggattctggagagctgtgcggcttcgccgccgtgggctacgccgcctggaaccggcggctgacagtggaagacatcgaggtcgctcctggccacagaggaagaggcatcggaagcgccctgatgggttgtgccgctgaattcggcagagagcggggcgccgagcacatctggctggaagtgtccagcgtgaacgcccctgccgtgcacgcctatagaagaatgggctttaccttctgcggcctggacaccgccctctacggcggcacccctgccgctggcgagcaggccctgttcatgtctagaccctgcagataa

SEQ ID NO: 51 - DNA, 576 bp, synthetic construct
  source 1..576: mol_type = other DNA
accaccgcacccggctccgccgacggcatcgtctaccgcccggcccgccccgaggacgccggcgccatcgaggccctggacagctccttcaccaccgccaccgtcttcgaggtgaccgtccacgccacgggcttcaccgtgcgcgaggtcccggtggacccgcccctgcgcaaggtgttcccgcccgaggagcacgacgagcaggcgctcggcggcggcgccccggactcggacggcgacgcgcgcacgtacgtggccctcgacggcggccgggtctgcggcttcgccgccgtcggctacaccccctggaaccgccggctgaccgtcgaggacatcgaggtcgcgcccggccaccgcgggcgcggcatcggccgcgcgctgatggagcacgccgccgacttcgcccgcgagcgcggcgcccggcacctgtggctggaggtcagcacggtcaacgccccggccgtgcacgcctaccggcggatggggttcaccctctgcgggctcgacaccacgctgtacgacgccaccccggccgcgggggagcgcgcgctgtacatgagccggccctgcggctaa

SEQ ID NO: 52 - DNA, 585 bp, synthetic construct
  source 1..585: mol_type = other DNA
accaccccacacggcccgggcggcgcagtcgtctaccgcctcgcccgccccgaggacgccggcgccatcgaggccctggacagctccttcaccacccccaccgtcttcgaggtggacgcctccggcgacggctgggggttcctgctccgggaagtccccgtcgacccgcccctgtacaaggtgttcccgcccgaggagcacggcgagcaggggtacgccggcgcccgggggcccgacgtggacgcggacacgcgcaccttcgtggccctcgacggcggcgagctgtgcgggttcgccgccctcggctacgccgcctggaaccggcggctgaccatcgaggacatcgcggtcgcgcccggccaccggggccgggggatcggcagcgccctgatggagcgtgccgccgacttcgcccgtgaacggggcgcggaacacctctggttggaggtcagctcggtcaacgcccccgccgtgcacgcctaccggcgcatgggattcaccctgtgcggcctcgacacggacctctacggcggcacgccctcggcgggcgagcgggccctgttcatgagccgcccctgccgctaa

SEQ ID NO: 53 - DNA, 585 bp, synthetic construct
  source 1..585: mol_type = other DNA
cctagcgccgacgacaccacctacgagatcagaacagccacccctgaggacgccggactcgtggaagccctggacggcagcttcaccacagcaaccattttccaggtggaaaccgctgagaatggatttaccctgcgggaaaccgctgtggaccctcccctgaccaaggtgttccccgacgaggaagatgagaacgttagcgccgatgaaggcgaccaggaacctcagggcgctcctaccttcctggctctggccccagatggcagcctggccggcttcgtgtccgtggcctacgagagatggaacagacggctgacaatcgaggacatcgaggtggcccctggccaccggggacgcggcgtgggcagagccctgatgagcagagccgaggaattcgccagagagcggggagccggccacatctggctggaagtgacaaacatcaacgcccctgccatccacgcctatagaagaatgggctttgccctgtgcggcctggatacatctctgtacgagttcacagcttctgccggcgagtacgccctgtacctgagcaagccatgtagaggcgccaaccgggactaa

SEQ ID NO: 54 - DNA, 384 bp, synthetic construct
  source 1..384: mol_type = other DNA
ccactgacccctgaagagacagccctggtggacgccgccacatctatcatcaccagcatccccatcagcgacacctacagcgtggccagcgccgctagatccacagacggcagaatcttcacaggcgtgaacgtgttccacttcaccggcggaccttgtgccgagctggttgtgctcggctctgccgctgctgccggcgccacccagctgacccacatcgtggccgtggctaatgagaaccggggaatcctgagcccctgcggccggtgcagacagaccctgatcgacctgcagcctggcattaaggtgatcgtgctggatagaggcgagcctagaggagtgcgggtggaagaactgctgccttttgcctacttcgccgattaa

SEQ ID NO: 55 - DNA, 315 bp, synthetic construct
  source 1..315: mol_type = other DNA
accaccctgagcttcgtggccgccaccgagctcgccgctacactgggagatgaccctaaccacaccgtggccgccgctgctctgggcctgaacggcaatatctacgccggcgtgaacaaccaccatttcaacggcggaccttgcgccgaactggtcgtgctgggcgttgcagctagagcccacgccggaaatctggccacaatggtggccgtgggcgacggcggcagaggcgtgatctctccatgtggccggtgcagacaggtgatgctggatcagcaccccgacatctgcgtgctggtgcctctggacgactaa

SEQ ID NO: 56 - DNA, 540 bp, synthetic construct
  source 1..540: mol_type = other DNA
ccattcgagcctctgtccgccacaggccagaacctgatcgacaccgccaccaccgtgatcaacaacatccccgtgtccgatttctacagcgtggccagcacagccatctctgatgacggcagagtgttcagcggcgtgaacgtgtaccacttcaccggcggaccttgtgccgagctggtgactctgggcgttgcagccgccgctggtgctcagaagctgacccacatcgtggccgtggctaatcaaaatagaggcatcctgagcccttgcggccggtgcagacaggtgctgaccgacctgcaccctggaatcaaggtggtcgtcgtgggcaaagagggcgccctgatgctgtggcctaggctgtacgcccagtggaagcacgccggcaagcccccctacctgattggatctagccactttggctgccagggcaacagcgtgtataagcacacccatgtgaaactgacaacaagcctgctgagcctgagagtgaaggccaccctgacacctcggaccaaggaactctactgcgaggaataa

SEQ ID NO: 57 - DNA, 540 bp, synthetic construct; identical to SEQ ID NO: 56
  source 1..540: mol_type = other DNA

SEQ ID NO: 58 - DNA, 423 bp, synthetic construct
  source 1..423: mol_type = other DNA
gcaaccatctacagccatctgtctgaggccgaacaaaatctgatcgaggtggccgctaaaacaatcgaggccatccccgtgtccgaggattatagcgttggatctgccgccctggccgaggacggcagaatcttcaccggcatcaacgtgtaccacttcaccggcggcccttgtgccgagctggtggtgctgggagtggctgctatggccggacctccaaagctgacccacatcgtggccgtcggcaaccagggcagaatgatcctgagcccttgcggccggtgcagacaggtgctcggcgacctgcaccccgacatcaaggccattgtgcgggacgccgatggcagcgtgaaggtggaaaaggtccaggacctgctgcctgccagatacgtgatccctgatgccacagtggaaagcatgtaa

SEQ ID NO: 59 - DNA, 426 bp, synthetic construct
  source 1..426: mol_type = other DNA
tctagcgccgcagatcaggccctgatcgagcgggccagagccctgattgagtccctgcccgacgacgagaaccacacagtggctgctgtggccctggacaccgccggccgccactttgacggcgttaatctgtatcacttcaccggcggactgtgcgccgagcctgtggtcctcgccgtggccgccgcccagcaggccgctcctctggaagtggtggtggccgtgggcaaccggggcagaggcgtgctggccccatgtggccggtgcagacagatcctgttcgactaccatcctgatatccaggtgctggtgccccacggacctcagatcagaagagtgggcatccgggaactgctgccttacacctacaactggcacgcccaaacagatagagagcacggcgaggccagcagacaggctgaataa

SEQ ID NO: 60 - DNA, 423 bp, synthetic construct
  source 1..423: mol_type = other DNA
gccaccatctacagccacctgagcaaggccgagcagaacctgatcgaggtggccacaaagaccatcgaggccattcctgtgtccgaggactattctgtcggaagcgccgccctggccgaggacggcagaatcttcaccggcatcaatgtgtaccacttcaccggcggtccttgcgccgaactggtggtgctgggcgtggccgctatggccggcccccccaagctgacacacatcgtggctgttggcaaccagggccgcatgatcctgtctccttgtggcagatgcagacaagtgctcggagatctgcatccagacatcaaggccatcgtgcgggtcgctgatggcagcgtgcgggtggaaaaagtgcaggacctgctgcctgccagatacgtgatccctgacaccacagtggaaagcatctaa

SEQ ID NO: 61 - DNA, 393 bp, synthetic construct
  source 1..393: mol_type = other DNA
gacctgacacctgaggaaatcaagctcgtggaagtggccaaggccaccatccagtccatttctacaagcgacacctacagcgtggcttctgccgccctgagcgccgacggaagaacattcagcggcgtgaacgtgttccactttaccggcggaccttgtgccgagctggtggtgctgggcagcgccgctggcgctaacgcccagaaactgaagaccatcgtggccgtgggagatgacggcgagaagggcgtggtgctgaacccctgcggccggtgcagacaggtgctgagagatctgcaacctagcatcaatgtggtcgtcgttaagggcggcaagctgaaaagcatctccatcaacgagctgctgccatacgcctacgacacccgggaataa

SEQ ID NO: 62 - DNA, 432 bp, synthetic construct
  source 1..432: mol_type = other DNA
gccgatctgcgggacctgtctgatgccgacttcgccctgatcgagcacgccagacagatcgtggaaagcaacggcgacggctctatcagcaccatgggcagcgccgctaggtccaccaccggcgagatctttggcgccattaacctgtaccatttcaccggaggaccttgtgccgagctggtggtcctgggcgtggctgccgcccacggcgtgcggagcctggaaacaatcgtggccgtgggtgacgagggcagaggccctgtgggaccttgcggccggtgcagacaagtgctgttcgactaccacccccagatcagagtgctcctgcctaccggcgctgagggagttaagagcgtggccatcggcgatctgctgccatacggcggcagatgggacgtggaactgggaacacagccttatgagcctacataa

SEQ ID NO: 63 - DNA, 393 bp, synthetic construct
  source 1..393: mol_type = other DNA
ggactgaacgccaaggaaaccaagctggttgacatcgccagagataccatcaacgccatcccccggagctctacacacagcgtgtccagcgccgctctgagcatcagcggccaggtctttaccgccgtgaacgtgttccatttcaccggcggcccttgcgccgagctggtggtgctcggcgtggctgctggcgccggaacacctcggctgtctcacatcgtggccgtgggcgaagatggccacgacggcatcatcctgaatccttgtggcagatgcagacaggtgctgtacgacctgcaccccggcattagagtgatcgtgcagaaaggcggaaaggccgagagcgtgctgatcgacgagctgctgccatacgcctacgagcctcgcgaataa

SEQ ID NO: 64 - DNA, 393 bp, synthetic construct
  source 1..393: mol_type = other DNA
gatctgacacctgaggaaaccaacctgatcgagatcgccagaaccaccatcaatgccatccccaagtctgatacctacagcgtggccagcgccgctctgagcgtggacggcagaatcttcaccggcgtgaacgtgtaccacttcacaggaggaccttgcgccgaactggtggtgctgggcgttgctgctggcgccggaacaccaagactgagccacatcgtggccatcggcgaagatggccaggacggcgtggtcctcaacccctgcggcaggtgtagacaggtgctgcacgacctgcatcctggcatcagagccattgtgcggaaggacggcgaggccaaatgcgtgtccatcaacgagctgctgccttggggctacggccctcgggactaa

SEQ ID NO: 65 - DNA, 390 bp, synthetic construct
  source 1..390: mol_type = other DNA
cccctccacgactccgaggtccggctgatcgacgcggccgaggcgctcgcccggacgctcggcgcggacccggaccacaccatggcggccgcggccctcgacgccgccggccggatccacgtcggcgtcaacgtcctgcacttcacgggcggcccgtgcgcggagctcgtcgcgctgggtgccgcggccgccgcgaatgcgggaccgctcgtggcgatggcggcggtgggcgacggcggccgggggatcgccccgccctgcggccggtgccgccaggtgatgctggatctccagcccggcatccgcgtggccgtgcccggcgccgacgggcccgagatcgtcgcgatccgcgacctgctgccggtctcgtacgcccgacccgacgcgtaa

SEQ ID NO: 66 - DNA, 516 bp, synthetic construct
  source 1..516: mol_type = other DNA
acgcgacgccacgagcccggtcctcggcccgagcccggctccgatcctggccctgagcccgagcccggccccggacccgagcccggacccggacccgagcccggacccgagcccggcccccgccccacccctcagcccgaccccggtcccgaccccgccctcgtgcgggccgccgccgcgctcgccgcgcggctcggggcggacgacaaccactcggtggccgcggcggcccgggacgccgggggccgggtggtcaccggtgtgaacgtgtaccacttcaccggcggcccctgtgccgaactcgtcgtgctgggcgccgccgtggccgagggcgcgggaccgctcgtgcggatcgtggcggtgggtgaccgggggcgcggggtgatgccgccgtgcggtcgatgccgacaggcgctgctcgacctgtggccgggtatcgaggtgctggtgccgggcgccgagggcggggtgcgtggcgtgcccgtgcgcgaactgctccctcatacatacgtgtaa

SEQ ID NO: 67 - DNA, 393 bp, synthetic construct
  source 1..393: mol_type = other DNA
accaccctaaccccccaagaagcctccctcctcgaaaccgccacaaaaacaataaccagcatcaaaccctccaacacgcacagcgtcgccagcgccgtcctcgcctccgacggccgcgtcttctccgccgtaaacgtctaccactttaccggcggcccttgtgccgaactcgtcgccctcgggaatgctgccgcggccggggccgaggagctcacccatatcgtggccgtcgaggatacccggcgtatcttgagtccctgtggacggtgtcggcaggttttgttggacttgtggcctggcattagggttattgttttgggggaagaggggcctagggttgttggcattgcggagttgttgccttttgcttattcgtggcctggggaggagtaa

SEQ ID NO: 68 - DNA, 390 bp, synthetic construct
  source 1..390: mol_type = other DNA
ccactgcacgacagcgaggccagactgatcgacgccgctgaagccctggcccggaccctcggcgctgatcctgatcacacaatggccgccgctgccctggatgccggaggcagaatccacgtgggcgtggacgtgctgcatttcaccggcggcccttgtgccgagctggttgccctcggcgccgccgctgcagaaaacgccggccccctggtggccatggccgctgtgggagatggcggaagaggcatcgtgcccccctgcggccggtgcagacaggtgatgctggacctgcagcctggcatccgggtggccgtgtctggcgccgacggccctgagatggtcggaatcggcgacctgctgcctgtgagctacgccagacctgacgagtaa

SEQ ID NO: 69 - DNA, 360 bp, synthetic construct
  source 1..360: mol_type = other DNA
cccctcacatcttctgaaaccaacctcgtaaacctagccatcaaggcaataacccaaatccccaaatcagaagactacagtgtctccagcgccgctctctcagaagacggccagatcttcaccggaataaatgtctaccacttcaccggcggcccctgtgcggaactcgtcacactgggcgtcgctgcgctcgccggacccccgaaactcactcttatcgtcgctgttagcaatgatggcaggatcctcagcccctgtggaagatgcagacaggtgctaagggatttgcatccgggtattaaagttattgttcctaaggaagggggcccggaagtggtggggattgatgatttgttgtaa

SEQ ID NO: 70 - DNA, 390 bp, synthetic construct
  source 1..390: mol_type = other DNA
ttagattataaggatatggaacttattgaaaaggcaagtgaaatattaaagaaaaattatgatagagaaaattacaatcatacagttgcagcagcagttaaatgtagtagtggtaatatatatttggggattaacgtattttctctacatggggcatgtgctgaacaagtggcaataggtactgctgttacaaacggagaaaaggattttaaatgtattgttgcaataagaggtgagaatggggatgaagtattatcaccatgtggaaattgtagacaaatgttatcagactattgtcctaactgtgaagtgattatacaaacaaatgatggattgcaaaaggtattagctaaagatttaataccttttgcatataaatctgaaagttaa

SEQ ID NO: 71 - DNA, 411 bp, synthetic construct
  source 1..411: mol_type = other DNA
accaccgggatccaccccgtcgaccacgaactcgtccgtgccgcgaccgacgtcgcgcgcacccggtgccggggcgacaaccacaccatggcggcagcggcccgtgcccgggacggccgcgtcatcacggccgtgaacgcctaccacttcaccggaggcccgtgcgccgaactggttctcatcggcacggccgccgcgcagggagcctacgaactggacaccgtcgtcgccgtgggcgaccgcgacaggggagtggtgccgccctgcggccgctgccgccaggtcctgctcgactacttcccctccctccgggtcatcgtcgggtccggcgaccgcctccgcgccgtccccgtgacggatctgctgccggacagctacgtctgggccgaccaccagccggacaccgactaa

SEQ ID NO: 72 - DNA, 357 bp, synthetic construct
  source 1..357: mol_type = other DNA
gatgctgcggaaaccctggcgcgaagcctcggcgacaacgacaatcacaccgttgcagcagcggcgatggacgttgatggacgcattcaccaagcagcgaatgtctaccacttcaccggtggtccgtgcgccgaactcgttgccttaggagttgcggccgctgcgggagcaaagcagcttttgactattgccgctgctggtgaccgagggcggggtttgattcctccatgtggtcgatgccgacaagttctcctcgatcatcatccggatattcttgtcgcggtccctgcggagaagggccctgtcgttcggcccgtccggaagctcctgccagtagtgccccgtagagtggtgtaa

SEQ ID NO: 73 - DNA, 579 bp, synthetic construct
  source 1..579: mol_type = other DNA
gacgtggacggcagaatccaccaggccgtgaacgtgtatcacttcaccggcggaccttgtgccgagctggtggccctcggcgttgctgccgccgctggcgccaagcagctgctcaccatcgccgccgctggagatagaggaagaggcctgatccctccttgcggccggtgcagacaggtgctgctggaacaccaccccgacatcctggtcgccgtgccagccgagaaaggccctgccgtgcgccccgtgcggaaactgctgctggacacctacttctaccctgacgcccaaggcaggagaatctttagattcaacaagcggtaccacgacgccgtgatctctggcgaaaagacaaccacaattagatgggacgagagcgtcgaggccggccccgctacatttgtgttcgaggaccaccctgagttcgcccatgtggaaggcgagatcatcagcgtgggtcagaccagactgcaggacctggatgccgaacgggccagaggcctgaaggcccactaccccagcatgcctgatgatgctgaactgagcagagtgtccttccgggtgcacggcgtgcggtaa

SEQ ID NO: 74 - protein, 45 aa, synthetic construct
SGGATNFSLLKQAGDVEENPGPSGGSGEGRGSLLTCGDVEENPGP

SEQ ID NO: 75 - DNA, 15 bp, synthetic construct
  source 1..15: mol_type = other DNA
acttcgtgtgcgagt

SEQ ID NO: 76 - DNA, 15 bp, synthetic construct
  source 1..15: mol_type = other DNA
cttcgtgtgcgagtc

SEQ ID NO: 77 - DNA, 15 bp, synthetic construct
  source 1..15: mol_type = other DNA
ttcgtgtgcgagtct

SEQ ID NO: 78 - DNA, 10 bp, synthetic construct
  source 1..10: mol_type = other DNA
tctgcgagtc

SEQ ID NO: 79 - DNA, 11 bp, synthetic construct
  source 1..11: mol_type = other DNA
tctgcgagtct

(19) United States
(12) Patent Application Publication
(10) Pub. No.: US 2025/0320483 A1
(43) Pub. Date: Oct. 16, 2025

(54) SYSTEMS AND METHODS FOR GENE INSERTIONS

(71) Applicant: The Trustees of Columbia University in the City of New York, New York, NY (US)

(72) Inventors: Alejandro Chavez, New York, NY (US); Joonwon Kim, New York, NY (US)

(21) Appl. No.: 19/191,835

(22) Filed: Apr. 28, 2025

Related U.S. Application Data
(63) Continuation of application No. PCT/US2023/078059, filed on Oct. 27, 2023.
(60) Provisional application No. 63/381,241, filed on Oct. 27, 2022.

(51) Int. Cl. C12N 15/10; C12N 9/22; C12N 15/11; C12N 15/86

(52) U.S. Cl. CPC C12N 15/102; C12N 9/226; C12N 15/111; C12N 15/86; C12N 2310/20; C12N 2740/15043; C12N 2840/203

(57) ABSTRACT

The present disclosure provides systems and methods for high throughput genetic manipulation. Particularly, systems and methods are provided for scalable gene insertions in mammalian cells, the systems and methods comprise a donor nucleic acid comprising a cargo sequence encoding one or more selectable markers; a first guide RNA complementary to at least a portion of the donor nucleic acid; a plurality of second guide RNAs each of which is complementary to at least a portion of one of a plurality of target nucleic acids; a first RNA-guided endonuclease configured to bind to the first guide RNA; a second RNA-guided endonuclease configured to bind to the plurality of second guide RNAs; or one or more nucleic acids encoding thereof.

### CROSS-REFERENCE TO RELATED APPLICATIONS

This application is a continuation of PCT International Application No. PCT/US2023/078059, filed Oct. 27, 2023, which claims the benefit of U.S. Provisional Application No. 63/381,241, filed Oct. 27, 2022, the contents of which are herein incorporated by reference in its entirety.

### STATEMENT REGARDING FEDERALLY SPONSORED RESEARCH OR DEVELOPMENT

This invention was made with government support under HG011855 awarded by the National Institutes of Health. The government has certain rights in the invention.

### FIELD

The present invention provides systems and methods for high throughput genetic manipulation. Particularly, systems and methods are provided for scalable gene insertions in mammalian cells.

### SEQUENCE LISTING STATEMENT

The content of the electronic sequence listing titled COLUM-41421-601.xml (Size: 108,111 bytes; and Date of Creation: Oct. 27, 2023) is herein incorporated by reference in its entirety.

### BACKGROUND

To obtain an accurate working model of the cell, the dynamic behavior and interaction partners for each of the thousands of proteins within it should be understood. A powerful approach to interrogate protein function is through the use of protein tags which are often appended to the C-terminus of a protein of interest, so as to minimize their influence on protein folding and localization. When fused to a protein these tags enable a myriad of studies such as the in vivo examination of protein localization (e.g., fluorescent protein tag), the affinity purification of a protein and its interaction partners from cells (e.g., FLAG epitope tag), or the rapid destruction of a target protein (e.g., FKBP-DD small molecule regulated degron tag).

As CRISPR/Cas9 has simplified the modification of mammalian genomes, there has been growing interest in tagging all human proteins at their endogenous loci to facilitate the comprehensive mapping of protein behavior. Homologous recombination has been used to insert tags at the C-terminus of target genes. Alternatively, non-homologous end joining has been used to insert synthetic exons containing protein tags into the introns of target genes. While powerful, these approaches still involve significant amounts of labor for each line generated or can perturb protein function due to the tag being inserted into the middle of the protein, respectively.

### SUMMARY OF THE INVENTION

Disclosed herein are systems and methods for scalable gene insertions.

In some embodiments, the present system and methods facilitate scalable gene tagging in mammalian cells (e.g., double and triple gene tagging, etc.). Accordingly, the present system and methods facilitate allow a large number (e.g., hundreds) of genes to be tagged within a similar time frame. For example, in some embodiments, the system and methods are used to tag genes at library scales. In some embodiments, the systems and methods find use in protein engineering. In some embodiments, the systems and methods find use in, e.g., adding an N or C-terminal protein tag (e.g., make a genome-wide library of cells that are YFP-tagged, degron-tagged, under inducible transcriptional control, FLAG-tagged, etc.), or enabling a promoter swap.

The systems and methods described herein are useful to modify one or more target sites on mammalian cell's genome. According to some embodiments, the systems and methods described herein are useful to edit, screen, label, mark or disrupt the genome of a mammalian cell. According to some embodiments, the systems and methods described herein are useful to insert exogenous DNA at one or more target sites on a mammalian cell's genome.

The systems and methods facilitate modifying a target site in a mammalian cell. In some embodiments, the systems and methods described herein facilitate modification of at least one target site in a population of mammalian cells. In some embodiments, the systems and methods described herein facilitate modification of a plurality of target sites in a mammalian cell. In some embodiments, the systems and methods described herein facilitate modification of a plurality of target sites in a mammalian cell population.

In some embodiments, the systems comprise: a donor nucleic acid comprising a cargo sequence encoding one or more selectable markers; a first guide RNA complementary to at least a portion of the donor nucleic acid, or a nucleic acid encoding thereof; a plurality of second guide RNAs each of which is complementary to at least a portion of one of a plurality of target nucleic acids, or one or more nucleic acids encoding the plurality of second guide RNAs; a first RNA-guided endonuclease, or a nucleic acid encoding thereof, configured to bind to the first guide RNA; and a second RNA-guided endonuclease, or a nucleic acid encoding thereof, configured to bind to the plurality of second guide RNAs.

In some embodiments, the one or more nucleic acid sequences encoding the one or more selectable markers are adjacent, individually or as a group, to one or more nucleic acid sequences encoding an internal ribosome entry site (IRES) or a ribosome skipping peptide. In some embodiments, the one or more nucleic acid sequences encoding the one or more selectable markers are operably linked to a promoter.

In some embodiments, the donor nucleic acid further encodes an insert. In some embodiments, the insert is a tag, a binding protein or domain thereof, an effector protein or domain thereof, a localization signal, a regulatory element, or a combination thereof.

In some embodiments, the cargo sequence encodes two or more selectable markers. In some embodiments, the one or more (e.g., two or more) selectable markers is individually selected from puromycin resistant genes, blasticidin resistant genes, and nourseothricin resistant genes. In some embodiments, each of the one or more selectable markers is a single marker type. In some embodiments, each of the one or more selectable markers is a different marker or marker type. In some embodiments, each of the one or more selectable markers is individually selected from the group in Table 1.

In some embodiments, the nucleic acid sequences encoding the one or more selectable markers are each individually adjacent to one or more nucleic acid sequences encoding an internal ribosome entry site (IRES) or a ribosome skipping peptide.

In some embodiments, the cargo sequence further encodes a transcription factor configured to activate the promoter operably linked to the one or more selectable markers.

In some embodiments, the plurality of second guide RNAs are in a plurality of cells, wherein each cell expresses a single second guide RNA complementary to at least a portion of one of the plurality of target nucleic acids.

In some embodiments, the first and/or second RNA-guided endonuclease is a Cas nuclease. In some embodiments, the first RNA-guided endonuclease and second RNA-guided endonuclease are orthogonal Cas nucleases. In some embodiments, the first and/or second RNA-guided endonuclease is a Cas9 nuclease.

In some embodiments, each of the Cas nucleases or Cas9 nucleases are individually derived from species in the group consisting of Streptococcus pyogenes, Staphylococcus aureus, Streptococcus thermophilus, Lactobacillus gasseri, Francisella novicida, Wolinella succinogenes, Sutterella wadsworthensis, gamma proteobacterium, Neisseria meningitidis, Camplyobacter jejuni, Fibrobacter succinogenes, Rhodobacter sphaeroides, Thermus thermophilus, Streptococcus thermophilus, and Rhodospirillum rubrum, or recombinant hybrids thereof. In some embodiments, one or both of the first and second RNA-guided endonuclease is a Cas9 ortholog individually selected from the group consisting of: Streptococcus pyogenes Cas9 (SpCas9), Staphylococcus aureus Cas9 (SaCas9), and Streptococcus thermophilus (StCas9). In some embodiments, one RNA-guided endonuclease is Streptococcus pyogenes Cas9 and one RNA-guided endonuclease is Staphylococcus aureus Cas9.

In some embodiments, the first and second RNA-guided endonuclease are encoded on a single nucleic acid.

In some embodiments, each of the plurality of target nucleic acids is in a cell. In some embodiments, the cell is a prokaryotic cell. In some embodiments, the cell is a eukaryotic cell. In some embodiments, the cell is a mammalian cell. In some embodiments, the cell is a human cell.

In some embodiments, one or more or all of the plurality of target nucleic acids encodes a gene or gene product. In some embodiments, one or more or all of the plurality of target nucleic acids encodes a protein or polypeptide. In some embodiments, the system is configured to insert the cargo sequence in frame with the gene product, protein, or polypeptide.

Also disclosed herein are methods for modifying one or more or all of a plurality of target nucleic acids comprising contacting a plurality of target nucleic acids with: a donor nucleic acid comprising a cargo sequence encoding one or more selectable markers; a first guide RNA complementary to at least a portion of the donor nucleic acid, or a nucleic acid encoding thereof; a plurality of second guide RNAs each of which is complementary to at least a portion of one of the plurality of target nucleic acids, or one or more nucleic acids encoding the plurality of second guide RNAs; a first RNA-guided endonuclease, or a nucleic acid encoding thereof, configured to bind to the first guide RNA; and a second RNA-guided endonuclease, or a nucleic acid encoding thereof, configured to bind to the plurality of second guide RNAs.

In some embodiments, one or more nucleic acid sequences encoding the one or more selectable markers are adjacent, individually or as a group, to one or more nucleic acid sequences encoding an internal ribosome entry site (IRES) or a ribosome skipping peptide. In some embodiments, one or more nucleic acid sequences encoding the one or more selectable markers are operably linked to a promoter.

In some embodiments, the cargo sequence further encodes an insert. In some embodiments, the insert is a tag, a binding protein or domain thereof, an effector protein or domain thereof, a localization signal, a regulatory element, or a combination thereof.

In some embodiments, the plurality of target nucleic acids are within a cell or cell population. In some embodiments, contacting a plurality of target nucleic acids comprises introducing into the cell or cell population.

In some embodiments, the cell or cell population is prokaryotic. In some embodiments, the cell or cell population is eukaryotic. In some embodiments, the cell or cell population is mammalian cells. In some embodiments, the cell or cell population is human cells.

In some embodiments, each cell in the cell population comprises a single second guide RNA.

In some embodiments, one or more or all of the plurality of target nucleic acids encodes a gene or gene product. In some embodiments, the target nucleic acid encodes a protein or polypeptide. In some embodiments, the system is configured to insert the cargo sequence in frame with the gene product.

Other aspects and embodiments of the disclosure will be apparent in light of the following detailed description.

### BRIEF DESCRIPTION OF THE FIGURES

FIGS. 1A-1G show the development of HITAG and properties of the generated lines. FIG. 1A is a schematic summary of HITAG. FIG. 1B is a graph of the distribution of gRNAs within the population of cells before tagging and after tagging and drug selection with the initial versus optimized HITAG approach. Each colored bar represents the abundance of one gRNA within the population. FIG. 1C is a graph of the tagging efficiency before drug selection as a function of different ng amounts of pCAS, pDNR-gRNA, and pDNR plasmid. Data shown are from three biological replicates (independent transfections), the error bars indicate ±the standard deviation. The optimal condition with an asterisk over it showed a significant difference (p<0.05) to all other conditions tested. FIG. 1D is a graph of the relative number of cells surviving puromycin selection when different donor plasmids with one or three copies of the selection marker were used, data are normalized to the P1 condition. Data shown are from three biological replicates (independent transfections), the error bars indicate ±the standard deviation, the donor with an asterisk over it showed a significant difference (p<0.05) to all other donors tested across targets. FIGS. 1E and 1F show the comparison of how RNA expression level (FIG. 1D), represented as log (FPKM+1) or the gRNA activity score (FIG. 1F) of each target-gRNAs influences if a gene is tagged or not within the pool of tagged HEK293T cells. FIG. 1G is the distribution of repair outcomes summed across all targets upon performing HITAG in HEK293T cells. Perfect junction indicates a precise annealing of the endogenous locus to the donor plasmid without a loss or addition of bases after Cas9 cutting. ****=p<10⁻⁴.

FIGS. 2A-2D show the use of HITAG to understand the properties of proteins that strongly gather within stress granules (SGs). FIG. 2A is images of the staining of proteins, showing robust accumulation within SGs after treatment with 0.5 mM of NaAs2O3 for 1 hour. (Blue: DAPI, Green: anti-G3BP1, Red: anti-mCherry). FIG. 2B is a list of proteins found to strongly accumulate within SG as determined by their overlap with the canonical SG marker, G3BP1. FIG. 2C is a network depicting the interactions among proteins which show robust accumulation within SGs. FIG. 2D is the predicted ability to liquid-liquid phase separation (LLPS) as determined by LLPS database (LLPSDB). As all proteins that showed strong accumulation were predominantly localized to the cytoplasm. For comparison cytoplasmic proteins were divided into those that strongly or weakly accumulate into SGs. As an additional group we included all other proteins that showed weak accumulation but did not primarily localize to the cytoplasm (non-cytosolic). ns=not significant; ****=p<10⁻⁴.

FIG. 3 is a schematic summary of CRISPaint approach for NHEJ-based gene tagging. Four different components-Cas9, target-gRNA, donor-gRNA, and donor plasmid—are transfected into cells for the CRISPaint approach. In CRISPaint, SpCas9 cleaves the target gene at its C-terminus and the donor plasmid (pDNR). Linearized pDNR can then become inserted into the genome through NHEJ. After transfection, the cells which are properly tagged in-frame will express mCherry fused to the protein of interest along with the puromycin resistance gene (PuroR), which enables properly tagged cells to grow in the presence of puromycin.

FIG. 4 is a schematic summary of HITAG approach for NHEJ-based high-throughput gene tagging. In the HITAG approach the target-gRNA is designed to interact with SpCas9, while the donor-gRNA is designed to interact with SaCas9. The library of target-gRNAs against the various genes of interest are first integrated into the pool of cells at low infectivity such that each cell gets on average a single target-gRNA. The remaining CRISPR components are then transfected in. This then enables each cell in the population to tag a unique gene to which their target-gRNA is against. When tagging occurs in-frame the protein of interest gains the C-terminal tag and a drug resistance marker (e.g., puromycin resistance gene) is also expressed, enabling properly tagged cells to be readily isolated via a round of drug selection.

FIGS. 5A-5B show how properly tagged genes can be recut when using only a single Cas9 protein. As shown in FIG. 5A, when using a single Cas9 protein, as is done in the original CRISPaint approach, the properly tagged product can be recut if the 3 PAM-proximal base pairs of the target gene are identical or similar to those of the donor plasmid. This occurs because the spacer sequence of the target-sgRNA with an accessible SpCas9-PAM site is regenerated in the final knock-in product. This recutting is then expected to inhibit the emergence of the perfectly tagged product without any errors at the junction site. As shown in FIG. 5B, recutting can be avoided when using two orthogonal Cas9 proteins, as is done in the HITAG approach This occurs because even if the final product that forms has homology to the spacer sequence of the target-gRNA it will have a SaCas9-PAM site proximal to it, and thus not be an appropriate substrate for SpCas9 which has different PAM requirements.

FIGS. 6A-6B show the identification of efficient and specific gRNAs against the donor plasmid. FIG. 6A is a graph of the efficiency of three different donor plasmid targeting gRNAs assessed for the ability to add a C-terminal mCherry tag to either CCND1, HIST1H4C, or PCNA. To measure efficiency, the percentage of cells with the appropriate mCherry localization as determined by fluorescence microscopy was quantified before the application of drug selection. Frame refers to the reading frame of the donor plasmid when cut. As there are three possible reading frames (e.g., 0, 1, and 2) a given target gene could be cut in, 3 donor plasmids for each donor-gRNA being tested were prepared. This enables the system to then cut the target gene and donor plasmid in such a way that should they undergo a perfect fusion (without the addition or removal of bases) the tag and drug marker will be in the appropriate reading frame. FIG. 6B is a graph of the same donor-gRNAs as in panel A examined for specificity by transfecting them in combination with SaCas9 and an mCherry-containing donor plasmid. As no target-gRNA is provided, all drug resistant clones that arise are due to low levels of stochastic double strand breaks or the donor-gRNA cutting an endogenous site in the genome to enable the donor plasmid to then insert itself. Based on these studies gRNA2 was used for all subsequent studies. Three biological replicates (independent transfections) were performed for all conditions and error bars represent #standard deviation.

FIG. 7 shows the comparison in tagging efficiency when using donor plasmids with varying numbers of the puromycin resistance gene. P1: single copy of puroR, P3: three copies of puroR, P3S: three copies of puroR but a stop codon is placed after the first puroR copy. Three biological replicates (independent transfections) were performed. Error bars represent ±standard deviation.

FIG. 8 shows the comparison in gRNA abundance between two independent rounds of HITAG on the same target-gRNA library. The abundance of each target-gRNA within the pool of cells was internally normalized between 0 to 1 for each replicate. p represents Spearman correlation coefficient.

FIG. 9 is a schematic summary of the results of tagging depending on the “frame” in which the target gene is cleaved by SpCas9. Here, frame number is defined as the number of nucleotides that must be added so that the target gene and the tag of interest plasmid are in-frame. In the original CRISPaint approach three unique donor-gRNA are used against a single donor plasmid to generate each of the needed reading frames, with each donor-gRNA potentially having its own set of off-target within the genome. In contrast, HITAG employs three different donor plasmids that all work with a single donor-gRNA. By designing each donor plasmid to have either 0, 1, or 2 bases added it enables the same donor-gRNA, which is tested to ensure it has minimal off-target activity, to be used across all studies. For example, pDNR (Frame 0) is used when tagging a target gene that does not require additional nucleotides added for the tag to be in-frame with the cut gene. Sequences shown: for pDNR Frame 0—SEQ ID NO: 75; for pDNR Frame 1—SEQ ID NO: 76; for pDNR Frame 2—SEQ ID NO: 77; Tagged gene (Frame 1)—SEQ ID NO: 78; and Tagged gene (Frame 2)—SEQ ID NO: 79.

FIG. 10 shows the percentage of genes tagged within each reading frame and as a whole across the generated HITAG libraries.

FIG. 11 shows the correlation between the normalized read counts from each gRNA within the pool of HITAG modified cells compared with the target-mCherry tag junction reads derived from the same pool of cells. p represents Spearman correlation coefficient between the two sets of data.

FIG. 12 shows the characterization of the junction between the target gene and mCherry tag. Target and Linker refer to the number of amino acids lost from the tagged gene or the linker which connects the tag to the gene, respectively. Insertion refers to the number of amino acids that are inserted between the target gene and the tag. Red dots indicate the junctions which show no deletion or insertion of additional amino acids.

FIGS. 13A-13D show the application of HITAG to HCT116 cells. The SG target-gRNA library 3 (frame 2) was integrated into HCT116 cells and the subsequent pool of cells was taken through the remainder of the HITAG procedure to isolate a mixed population of mCherry tagged cells. FIG. 13A shows the correlation between the normalized read counts from each gRNA within the pool of HITAG modified HCT116 cells compared with the target-mCherry tag junction reads derived from the same pool of tagged HCT116 cells. FIG. 13B shows the distribution of repair outcomes summed across all targets upon performing HITAG in HCT116 cells. Perfect junction indicates a precise annealing of the endogenous locus to the donor plasmid without a loss or addition of bases after Cas9 cutting. FIGS. 13C-13D show the comparison of how RNA expression level (FIG. 13C) represented as log (FPKM+1) or the on-target efficacy score of target-gRNAs (FIG. 13D) influence if a gene is tagged or not within the pool of tagged HCT116 cells. ***=p<10⁻³

FIG. 14 shows the number of times a given gene was tagged within the set of 806 clonal lines that were isolated after performing HITAG. The yellow bar represents the median number of clones obtained for a given targeted gene.

FIGS. 15A-15B show the examination of the rates of off-target tagging by quantifying the consistency of results when multiple clones of the same tagged gene were obtained. FIG. 15A is the proteins with either nuclear or ER localization which had 10 or more clones to examine from within the 807 clonal isolates studied to determine the number of clones that showed the appropriate localization. FIG. 15B is PCR validation using primers to show that microscopy-based results were concordant with targeted PCR directed at the junction between either HNRNPA2B1 or BCLAF1 and the mCherry tag. C: control DNA from untargeted HEK293T cells. L: Ladder.

FIG. 16 shows the decision tree used to compare localization information between this study and the human protein atlas. Protein subcellular localization information was available for 155 out of 167 genes in Human Protein Atlas database. 141 out of 155 genes showed a similar subcellular localization as described in Human Protein Atlas. Of the 14 genes that disagreed with the data from Human Protein Atlas, 6 genes were found in Opencell or RBP Image Database. 5 out of 6 of these genes agreed with our subcellular localization findings.

FIG. 17 shows the comparison of protein localization and dynamics between mCherry-tagged version and its endogenous untagged counterpart. Images of stained cells under both homeostatic and stressed (arsenite) conditions are shown.

FIGS. 18A-18L show the analysis of the features which drive strong accumulation within SGs. Because all proteins that strongly gather in SG were cytosolic analyses are done comparing cytosolic proteins that show strong vs weak accumulation. As an additional category proteins that show non-cytosolic localization are also examined. LLPS scores from CatGranule (FIG. 18A), LLPS scores from Plaac (FIG. 18B), Protein length (number of amino acid) (FIG. 18C), Number of intrinsically disorder regions (FIG. 18D), RNA expression level represented as log (FPKM+1) (FIG. 18E), protein abundance extracted from mass spectrometry data (FIG. 18F), fraction of charged residues (FIGS. 18G-18I), net charge per residues at pH 7.4 (FIG. 18J), absolute mean net charge at pH 7.4 (FIG. 18K), and Uversky hydropathy (FIG. 18L) were compared the groups. (ns: not significant; *: p<0.05; **: p<10⁻²; ***: p<10⁻³; ****: p<10⁻⁴)

FIGS. 19A-19C show use of different puromycin resistance genes in HITAG. FIG. 19A shows metagenomic analysis of puromycin resistance gene homologs. In FIG. 19B, donor constructs containing new puromycin resistance markers were used in HITAG and cells resistant to puromycin were visualized by microscopy. The change in media color indicates increased cell growth (e.g., yellow: high cell growth, red: low cell growth). FIG. 19C shows the distribution of gRNAs within the population of cells after tagging and drug selection using two top performing puromycin resistance markers RaPuroR and PfPuroR. Different colors in the bars represent the relative proportion of a given gRNA in the tagged pool of cells.

FIGS. 20A-20B show design and use of a drug circuit to enhance selection. FIG. 20A is a schematic of the original puromycin resistance construct (top) and a schematic of a drug circuit in which a transcription factor is produced from the tagged gene and this then binds to a promoter driving puromycin resistance (amplifying the signal).

FIGS. 21A-21C show the effects of peptide skipping peptides in the donor construct. FIG. 21A is a western blot showing the presence of the higher molecular weight species with a ˜25 kDa shift in size from the simple 3×FLAG fusion using a single skipping peptide (T2A). By placing two copies of the skipping sequence (PT2A), this higher molecular weight species (marked by red or yellow arrows and boxes) was removed and a marked increase in the 3×FLAG tagged product is observed. Amino acid sequence of PT2A is: SGGATNFSLLKQAGDVEENPGPSGGSGEGRGSLLTCGDVEENPGP (SEQ ID NO: 74). FIG. 21B is a diagram illustrating that a single skipping peptide often produces a chimeric protein between the drug marker and the tagged protein of interest along with a comparison between the original T2A and PT2A design. FIG. 21C is the tagging efficiency for the single skipping peptide (T2A) and the PT2A construct.

FIGS. 22A-22C show an exemplary use of the disclosed methods to modify multiple alleles. FIG. 22A is a schematic for a modified tagging pipeline where two rounds of tagging occur. FIG. 22B shows the products of dual allele tagging against a library of targets. FIG. 22C shows the usage of dual allele tagging approach to fuse 3×FLAG to the C-terminus of two different alleles of the gene YY1 in the same cell line. “Two allele tagging” is from HEK293T cells exposed to two rounds of tagging with the first round containing a pDNR plasmid with a puromycin resistance marker (as in “one allele tagging”) and the second round of tagging containing a pDNR with a nourseothricin resistance marker. To check for dual allele tagging PCR was performed looking for PCR junctions between the various drug markers and the C-terminus of the target gene YY1. P=puromycin resistance gene-C-terminus of YY1 junction being amplified by PCR; N=nourseothricin resistance gene-C-terminus of YY1 junction being amplified by PCR.

### DETAILED DESCRIPTION

Although numerous methods exist for performing targeted knockins into the mammalian genome, most allow only a single protein at a time to be targeted, are inefficient, or place tags in between coding exons which can disrupt protein folding and function. To solve the need for a high-throughput method of gene tagging that would minimally perturb protein function, High-throughput Insertion of Tags Across the Genome (HITAG) was developed. HITAG uses a Cas protein (e.g., Cas9) in combination with non-homologous end joining (NHEJ) to insert protein tags into the C-terminus of target genes. The HITAG process occurs within a mixed pool of cells wherein at the end of the procedure each cell ends up with a distinct protein C-terminally tagged. In analyzing the insertion events mediated by HITAG, over 70% were found to be “perfect” fusion between the tag and the target gene without the insertion or deletion of additional bases. To enable HITAG, development of a modified selection marker (e.g., multiple copies of marker, different markers, marker circuit to increase transcription/translation of marker(s), and/or multiple copies of skipping peptides) enabled the efficient enrichment of cell with the proper in-frame insertion from the initial mixed pool. Overall, the modified marker HITAG facilitates the scalable interrogation of protein function and dynamics.

HITAG finds use in a variety of applications in which libraries of tagged genes are utilized, including, for example, interrogation of protein function (e.g., HITAG used in combinations with single cell chromatin immunoprecipitation (ChIP) assays with sequencing, ChIP sequencing (ChIP-Seq), e.g., to map transcription factor binding at scale, and HITAG linked to degron analysis to probe regulatory networks), identification of protein localization and interaction partners (e.g., to build an interaction network, e.g., to predict genes required for disease etiology), generation large quantities of protein functions (e.g., new CRISPR effectors (e.g., activators, base editors, prime editors, inhibitors), and exploration of the effects of induced protein-protein interactions by labeling two proteins with binding partners or recruitment system components.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46:453 (1960) and Doty et al., Proc. Natl. Acad. Sci. USA, 46:461 (1960), have been followed by the refinement of this process into an essential tool of modern biology. For example, hybridization and washing conditions are now well known and exemplified in Sambrook et al., supra. The conditions of temperature and ionic strength determine the “stringency” of the hybridization.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity.

As used herein, the terms “providing,” “administering,” and “introducing,” are used interchangeably herein and refer to the placement into a cell, organism, or subject by a method or route which results in at least partial localization to a desired site. Administration can be by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Systems

Disclosed herein are systems for modifying a plurality of target nucleic acids. The systems may be used for scalable (e.g., library scales) gene insertions, for example for use in protein engineering (e.g., to add an N- or C-terminal tag, moiety, or domain to one or more proteins) or promoter engineering (e.g., to introduce or substitute regulatory elements).

The target nucleic acids may be in vitro or in a cell. In some embodiments, a target nucleic acid is a nucleic acid endogenous to a target cell. In some embodiments, a target nucleic acid is a genomic DNA sequence. The term “genomic,” as used herein, refers to a nucleic acid sequence (e.g., a gene or locus) that is located on a chromosome in a cell.

In some embodiments, a target nucleic acid encodes a gene or gene product. The term “gene product,” as used herein, refers to any biochemical product resulting from expression of a gene. Gene products may be RNA or protein. RNA gene products include non-coding RNA, such as tRNA, IRNA, micro RNA (miRNA), and small interfering RNA (siRNA), and coding RNA, such as messenger RNA (mRNA). In some embodiments, a target nucleic acid sequence encodes a protein or polypeptide. In some embodiments, the systems facilitate an insertion in frame with the gene product.

In some embodiments, the systems comprise at least one or all of: a donor nucleic acid comprising a cargo sequence, a first guide RNA complementary to at least a portion of the donor nucleic acid, a plurality of second guide RNAs each of which is complementary to at least a portion of one of a plurality of target nucleic acids, a first RNA-guided endonuclease configured to bind to the first guide RNA, and a second RNA-guided endonuclease configured to bind to the second guide RNA; or one or more nucleic acids encoding any of the listed components.

In some embodiments, the cargo sequence encodes one or more (e.g., one, two, three, four, five, six, seven, eight, nine, ten, or more) selectable markers. In some embodiments, the cargo sequence encodes two or more selectable markers. As used herein, “selectable marker” means a nucleotide sequence that when expressed imparts a distinct phenotype to the host cell expressing the marker and thus allows such transformed cells to be distinguished from those that do not have the marker. Such a nucleotide sequence can encode either a selectable or screenable marker, depending on whether the marker confers a trait that can be selected for by chemical means, such as by using a selective agent (e.g., an antibiotic and the like), or whether the marker is simply a trait that one can identify through observation or testing, such as by screening (e.g., fluorescence). Of course, many examples of suitable selectable markers are known in the art and can be used in the vectors described herein.

Each of the one or more or two or more selectable markers may be the same, each may be a different type of selectable marker, or a combination thereof. For example, each of the selectable markers may confer resistance to the same antibiotic. Alternatively, each of the selectable markers may confer resistance to a different antibiotic, or one may confer resistance to an antibiotic and one may result in a colorimetric observation (e.g., a fluorescent marker). In select embodiments, each of the selectable markers is the same type of market. In select embodiments, each of the selectable markers confers resistance to the same antibiotic.

In some embodiments, each of the one or more selectable markers is individually selected from puromycin resistant genes, blasticidin resistant genes, and nourseothricin resistant genes. In select embodiments, the selectable markers are individually selected from the group in Table 1. In some embodiments, at least one of the one or more selectable markers is a puromycin resistant gene, blasticidin resistant gene, or a nourseothricin resistant gene. In some embodiments, at least one of the one or more selectable markers is selected from the group in Table 1.

In some embodiments, the nucleic acid sequence(s) encoding the one or more selectable markers are adjacent (e.g., immediately adjacent or contiguous or separated by one or more linker nucleotides), individually or as a group, to one or more (e.g., one, two, three, four, five, or more) nucleic acid sequences encoding an internal ribosome entry site (IRES) or a ribosome skipping peptide. For example, the nucleic acid sequence(s) encoding two or more selectable markers may be adjacent to each other and preceded or followed by one or more nucleic acid sequences encoding an internal ribosome entry site (IRES) or a ribosome skipping peptide. Alternatively, the nucleic acid sequence(s) encoding two or more selectable markers may each be preceded and/or followed by one or more nucleic acid sequences encoding an internal ribosome entry site (IRES) or a ribosome skipping peptide. In some embodiments, a nucleic acid sequence for two or more internal ribosome entry sites or ribosome skipping peptides may be adjacent to the selection marker.

Internal ribosome entry sites (IRESs) or ribosome skipping peptides assist in the co-translation of multiple independent polypeptides from a single transcript. The ribosome skipping peptide may be a 2A family peptide. 2A peptides are short (˜18-25 aa) peptides derived from viruses. There are four commonly used 2A peptides, P2A, T2A, E2A and F2A, that are derived from four different viruses. Any known 2A peptide sequence is suitable for use in the disclosed system.

The selectable marker(s) may be preceded or followed by the one or more IRES or ribosome skipping peptide based on the relationship to the gene product at the location of the target nucleic acid following insertion. When the selectable marker(s) are upstream of the gene product following insertion the one or more IRES or ribosome skipping peptide may be downstream of the selectable marker(s), whereas when the selectable marker(s) are downstream of the gene product following insertion one or more IRES or ribosome skipping peptide may be upstream. Thus, in either instance, following translation two separate products are produced: the gene product and the selectable marker product. For example, when two or more selectable markers are included, each one may be preceded or followed by one or more IRES or ribosome skipping peptide. In some embodiments, when two or more ribosome skipping peptides are used the nucleic acid sequence encodes a peptide comprising an amino acid sequence of SGGATNFSLLKQAGDVEENPGPSGGSGEGRGSLLTCGDVEENPGP (SEQ ID NO: 74).

In some embodiments, the nucleic acid sequence(s) encoding the one or more selectable markers are operably linked to a promoter. In such instances, the selectable marker is separately transcribed, and thus separately translated, from the gene product following insertion.

In some embodiments, the cargo sequence further encodes a transcription factor configured to activate the promoter operably linked to the one or more selectable markers. The nucleic acid sequence encoding the transcription factor may be adjacent, upstream or downstream, to one or more nucleic acid sequences encoding an internal ribosome entry site (IRES) or a ribosome skipping peptide, as described above for the selectable markers.

In some embodiments, the donor nucleic acid further encodes at least one insert. The insert is the element with which the target nucleic acid (e.g., gene or gene product) is being modified. In some embodiments, the insert is selected from the group consisting of a tag, a binding protein or domain thereof, an effector protein or domain thereof, a localization signal, a regulatory element, or a combination thereof.

The tag includes any tag useful in identifying a gene product, in vivo or in vitro. Exemplary tags include, but are not limited to, an antibody tag (e.g., human influenza hemagglutinin (HA), and the like), antibody-epitope tag (a Myc tag, a VS tag, and the like), fluorescent protein tag (e.g., GFP, YFP, RFP, mNeonGreen, TdTomato, and the like), an affinity purification tag (e.g., a Biotin tag, a His tag, and the like), a stability tag (e.g., degron, chemically stabilized FKBP variants, PEST domain, and the like), and the like.

The binding protein or domain thereof includes proteins, domain, or moieties which result in conferring the gene or gene product (e.g., protein) with a binding capability not naturally associated with the gene or gene product. For example, the binding protein or domain thereof includes but is not limited to a protein-protein interaction domain, a chemically induced protein-protein interaction domain, a nucleic acid binding domain.

The effector protein or domain thereof includes proteins, domain, or moieties which result in conferring the gene or gene product (e.g., protein) with a functionality (e.g., enzymatic functionality) not naturally associated with the gene or gene product. The effector protein or domain thereof may comprise a number of functionalities, including but not limited to, nuclease function, recombinase function, epigenetic modifying function, transposase function, integrase function, resolvase function, invertase function, protease function, DNA methyltransferase function, DNA demethylase function, histone acetylase function, histone deacetylase function, transcriptional repressor function, transcriptional activator function, DNA binding protein function, transcription factor recruiting protein function, nuclear-localization signal function, DNA editing function (e.g., deaminase) or any combination thereof. For example, some effector proteins or domains thereof function in transcriptional regulation via their ability to interact with the basal transcriptional machinery and general co-activators, interact with other transcription factors to allow cooperative binding, and/or directly or indirectly recruit histone and chromatin modifying enzymes.

Localization signals are peptide sequences or protein domains that designate a protein for translocation to a certain organelle or sub-cellular compartment (e.g., nucleus, cytoplasm, membrane, periplasm, or for secretion outside of the cell). For example, nuclear localization sequences usually comprises one or more positively charged amino acids, such as lysine and arginine. Other localization signals include, but are not limited to, ER-retention sequence, plasma membrane localization sequence, and the like

Regulatory elements include sequences involved in modulating transcription (e.g., promoters, enhancers, silencers, and insulators, Kozak sequences, and introns) and translation of a gene.

The system comprises a first guide RNA complementary to at least a portion of the donor nucleic acid and a plurality of second guide RNAs each of which is complementary to at least a portion of one of a plurality of target nucleic acids, or one or more nucleic acids encoding the first guide RNA or the plurality of second guide RNAs. Each of the first guide RNA and the plurality of second guide RNAs form a complex with the RNA-guided endonuclease and directs the cleavage of the respective nucleic acids to which they are hybridized.

The first guide RNA hybridizes to the donor nucleic acid. When the donor nucleic acid is provided as a vector, the first guide RNA hybridizes to the target site and directs cleavage of the vector creating a linear insert for the donor nucleic acid and its cargo. The system may include a plurality of first guide RNAs targeting a single site within the donor nucleic acid or different sites with the donor nucleic acid. In some embodiments, the system comprises more than one first guide RNA which hybridize at unique sites within the donor nucleic acid. The different sites may be at different locations relative to the cargo, e.g., flanking the cargo, 3′ of the cargo, or 5′ of the cargo.

The present systems include a plurality of second guide RNAs. In some embodiments, the plurality of second guide RNAs include guide RNAs that target one or more different target genes or target gene specific sequences. For example, the second guide RNAs can bind to different target genes, e.g., to facilitate insertion at multiple different target genes. Alternatively, the second guide RNAs can target gene specific sequences, e.g., to facilitate insertion at different locations within a single target gene. In select embodiments, the plurality of second guide RNAs is at least partially complementary to multiple (e.g., tens, hundreds, or thousands of) different target genes.

Each of the plurality of second guide RNAs can target at least one region of the target nucleic acid (e.g., target gene). For example, the guide RNA may bind and hybridize to a region of a target gene selected from: a promoter region, an enhancer region, a repressor region, an insulator region, a silencer region, a region involved in DNA looping with the promoter region, a gene splicing region, or a transcribed region. The second guide RNAs can target a sequence of the target gene, such that the endonuclease will cleave in the reading frame (e.g., the transcribed region) of the target gene.

In some embodiments, the plurality of second guide RNAs are in a plurality of cells, wherein each cell expresses a single second guide RNA. The population of cells cover a plurality of target nucleic acids, with each cell comprising a single second guide RNA to a single target nucleic acid. Thus, the system may comprise a plurality of cells each comprising a single second guide RNA.

The first guide RNA and the plurality of second guide RNAs may individually be a crRNA, crRNA/tracrRNA (or single guide RNA, sgRNA). The terms “gRNA,” “guide RNA,” “crRNA,” and “CRISPR guide sequence” may be used interchangeably throughout and refer to a nucleic acid comprising a sequence that determines the binding specificity of the RNA-guided endonucleases in the system. A gRNA hybridizes to (complementary to, partially or completely) a target site (e.g., on the donor nucleic acid or on the target nucleic acid). In some embodiments, the at least one gRNA is encoded in a CRISPR RNA (crRNA) array.

The first guide RNA and the plurality of second guide RNAs or portion thereof that hybridizes to the target site may be any length. In some embodiments, the gRNA sequence that hybridizes to the target site is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLOS ONE, 10 (3): (2015)); Zhu et al. (PLOS ONE, 9 (9) (2014)); Xiao et al. (Bioinformatics. January 21 (2014)); Heigwer et al. (Nat Methods, 11 (2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to the target site, in some embodiments, the first guide RNA and/or the plurality of second guide RNAs may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337 (6096): 816-821, and Ran, et al. Nature Protocols (2013) 8:2281-2308, incorporated herein by reference in their entireties.

In some embodiments, the first guide RNA and/or the plurality of second guide RNAs does not comprise a scaffold sequence and a scaffold sequence is expressed as a separate transcript. In such embodiments, the first guide RNA and/or the plurality of second guide RNAs further comprises an additional sequence that is complementary to a portion of the scaffold sequence and functions to bind (hybridize) the scaffold sequence.

The first guide RNA and/or the plurality of second guide RNAs can comprise spacer sequence. The spacer sequence can be any length. In some embodiments, the spacer sequence is 30-40 nucleotides long (e.g., 30, 31, 32, 33, 34, 35, 36, 37, 38, 39, or 40).

In some embodiments, the first guide RNA and/or the plurality of second guide RNAs is at least 50%, 55%, 60%, 65%, 70%, 75%, 80%, 85%, 90%, 95%, 96%, 97%, 98%, 99%, or at least 100% complementary to a target nucleic acid. In some embodiments, the first guide RNA and/or the plurality of second guide RNAs is at least 50%, 55%, 60%, 65%, 70%, 75%, 80%, 85%, 90%, 95%, 96%, 97%, 98%, 99%, or at least 100% complementary to the 3′ end of the target site (e.g., the last 5, 6, 7, 8, 9, or 10 nucleotides of the 3′ end of the target site).

“Target site” refers to a polynucleotide (nucleic acid, gene, chromosome, genome, etc.) to which a guide sequence (e.g., a gRNA) is designed to have complementarity, wherein hybridization between the target site sequence and a guide sequence promotes the formation of a complex with the RNA guided endonuclease, provided sufficient conditions for binding exist. The target site sequence and guide sequence need not exhibit complete complementarity, provided that there is sufficient complementarity to cause hybridization and promote formation of the complex.

The target site sequence may or may not be flanked by a protospacer adjacent motif (PAM) sequence. In certain embodiments, an RNA-guided nucleases can only cleave a target site sequence if an appropriate PAM is present, see, for example Doudna et al., Science, 2014, 346 (6213): 1258096, incorporated herein by reference. A PAM can be 5′ or 3′ of a target sequence. A PAM can be upstream or downstream of a target site sequence. In one embodiment, the target site sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target site sequence may or may not be located adjacent to a PAM sequence (e.g., PAM sequence located immediately 3′ of the target sequence). Non-limiting examples of PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, NGGNG and NNAGAAW (W=A or T), NNNNGATT, NAAR (R=A or G), NNGRR (R=A or G), NNAGAA, and NAAAAC, where N is any nucleotide.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

The system comprises a first RNA-guided endonuclease configured to bind to the first guide RNA and a second RNA-guided endonuclease configured to bind to the plurality of second guide RNAs, or one or more nucleic acids encoding the first and second RNA-guided endonucleases. In some embodiments, the first and second RNA-guided endonuclease are encoded on a single nucleic acid. In some embodiments, the first and second RNA-guided endonuclease are encoded on separate nucleic acids.

RNA-guided endonucleases are nucleases which form a complex with a nucleic acid, usually RNA, which provides the target sequence specificity for the endonuclease. Once the nucleic acid is complexed with the RNA-guided endonuclease and has recognized and hybridized to the target site, the RNA-guided endonuclease cleaves the target nucleic acid. RNA-guided endonucleases include argonaute proteins, CRISPR (Clustered Regularly Interspaced Short Palindromic Repeats)-associated (Cas) proteins, CRISPR-associated transposase proteins, and OMEGA (Obligate Mobile Element Guided Activity) system proteins. In addition, synthetic or engineered RNA-guided nucleases are also applicable to the system disclosed herein. See, for example, Schmidt, M. J., et al. Nat Commun 12, 4219 (2021).

In some embodiments, the first and second RNA-guided endonuclease are orthogonal RNA-guided endonucleases. As used herein in connection with the RNA-guided endonucleases, the term “orthogonal” means that the RNA-guided endonucleases indicated to be orthogonal to each other do not bind at a significant level to the same binding pair member, e.g., they recognize different binding sites on different molecules. In some embodiments, orthogonal RNA-guided endonucleases do not bind the same gRNAs due to different binding sequences on the gRNAs which only interact with one of the RNA-guided endonuclease. Thus, the first RNA-guided endonuclease interacts with the first guide RNA and the second RNA-guided endonuclease interacts with the plurality of second guide RNAs.

In some embodiments, the first and/or second RNA-guided endonuclease is a Cas nuclease, or a functional fragment or variant thereof. The Cas nuclease can be obtained from any suitable microorganism, and a number of bacteria express Cas protein orthologs or variants. Cas9 nuclease of other species are known in the art (see, e.g., U.S. Patent Application Publication 2017/0051312, incorporated herein by reference) and may be used in connection with the present system. The amino acid sequences of Cas nucleases from a variety of species are publicly available through the GenBank and UniProt databases. The Cas nuclease may be from Streptococcus pyogenes, Staphylococcus aureus, Streptococcus thermophilus, Lactobacillus gasseri, Francisella novicida, Wolinella succinogenes, Sutterella wadsworthensis, gamma proteobacterium, Neisseria meningitidis, Camplyobacter jejuni, Fibrobacter succinogenes, Rhodobacter sphaeroides, Thermus thermophilus, Streptococcus thermophilus, or Rhodospirillum rubrum.

In some embodiments, the Cas nuclease is Cas9, or a functional fragment or variant thereof. In some embodiments, the Cas9 nuclease is from Streptococcus pyogenes or Staphylococcus aureus. In some embodiments, each of the Cas9 nucleases are individually selected from Streptococcus pyogenes Cas9 (SpCas9), Staphylococcus aureus Cas9 (SaCas9), and Streptococcus thermophilus (StCas9). In select embodiments, one Cas nuclease is Streptococcus pyogenes Cas9 (SpCas9) and one Cas nuclease is Staphylococcus aureus Cas9 (SaCas9).

Cas nuclease variants having alterations in the PAM requirements of target nucleic acids; decreased off-target binding or increase on-target binding; and the like are suitable for use in the disclosed systems. For example, Streptococcus pyogenes Cas 9 (SpCas9) variants SpCas9-VQR, -VRQR, -EQR, -VRER, xCas9, SpCas9-NG, SpG, and SaKKHn allow targeting of genomic regions containing non-NGG PAMs and SpRY is a near-PAMless variant of SpCas9 (See, Kleinstiver B P et al., Nature. 523, 481-5 (2015); Kleinstiver B P et al., Nature. 529, 490-5 (2016); Kim et al., 2017, Nat. Biotechnol. 35, 371-376; Nishimasu, H. et al., 2018, Science 361, 1259-1262; Hu J H, et al., Nature. 556, 57-63 (2018); Miller, et al., 2020, Nat. Biotechnol. 38, 471-481; Yang, L. et al., 2018, Protein Cell 9, 814-819, Walton, et al., 2020, Science 268, 290-296, incorporated herein by reference).

### Nucleic Acids and Delivery

The present disclosure also provides for nucleic acids encoding the components of the disclosed systems and vectors containing or encoding these nucleic acids. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more components of the disclosed systems. The vector(s) can be introduced into a cell that is capable of expressing a protein, polypeptide, or gRNA encoded thereby, including any suitable prokaryotic or eukaryotic cell.

In some embodiments, the donor DNA may be on a single vector, separate from any other components of the disclosed system and methods. In some embodiments, the first and second RNA-guided endonucleases are included on the same vector. This vector may include any one or more additional components of the disclosed systems (e.g., the first and second guide RNAs). In some embodiments, the first and second guide RNAs are included on the same vector. In some embodiments, the first and second guide RNAs are included on different vectors, separate from any one or more additional components of the disclosed systems.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the eukaryotic cell and/or cells derived from the subject are returned to the subject.

Viral and non-viral based gene transfer methods can be used to introduce nucleic acids encoding the components of the disclosed systems into cells, tissues, or a subject. Such methods can be used to administer nucleic acids encoding the disclosed polypeptides or components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. Drug selection strategies may be adopted for positively selecting for cells. A nucleic acid may contain one or more drug-selectable markers.

A variety of viral constructs may be used to deliver the components of the present system to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7 (1): 33-40; and Walther W. and Stein U., 2000 Drugs, 60 (2): 249-71, incorporated herein by reference.

In one embodiment, a nucleic acid encoding the components of the disclosed systems is contained in a plasmid vector that allows expression of the components of the disclosed systems and subsequent isolation and purification of from the recombinant vector. Accordingly, the components of the disclosed systems can be purified following expression, obtained by chemical synthesis, or obtained by recombinant methods.

To construct cells that express the components of the disclosed systems, expression vectors for stable or transient expression of the components of the disclosed systems may be constructed via conventional methods as described herein and introduced into host cells. For example, nucleic acids encoding the components of the disclosed systems may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like β-globin or β-globin; SV40 polyoma origins of replication and ColE1 for proper episomal replication; internal ribosome binding sites (IRES), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

The components of the disclosed systems may be delivered by any suitable means. In certain embodiments, the components of the disclosed systems are delivered in vivo. In other embodiments, the components of the disclosed systems are delivered to isolated/cultured cells (e.g., autologous iPS cells) in vitro to provide modified cells.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the disclosed systems is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110 (6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding any one or more of the components of the disclosed systems is a DNA molecule. In some embodiments, the nucleic acid encoding any one or more of the components of the disclosed systems is a DNA vector and may be electroporated to cells. In some embodiments, the nucleic acid encoding any one or more of the components of the disclosed systems is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459 (1-2): 70-83), incorporated herein by reference.

### Methods

Also disclosed herein are methods for nucleic acid modification. The phrase “modifying a nucleic acid sequence” or “nucleic acid modification” as used herein, refers to modifying at least one physical feature of a nucleic acid sequence of interest. Nucleic acid modifications include, for example, single or double strand breaks, deletion, and/or insertion of one or more nucleotides, and other modifications that affect the structural integrity or nucleotide sequence of the nucleic acid sequence. In some embodiments, the methods facilitate inserting an exogenous nucleic acid at a target site in the nucleic acid of interest.

In some embodiments, the methods comprise contacting a plurality of target nucleic acids with: a donor nucleic acid; a first guide RNA complementary to at least a portion of the donor nucleic acid or a nucleic acid encoding the first guide RNA; a plurality of second guide RNAs each of which is complementary to at least a portion of one of a plurality of target nucleic acids, or one or more nucleic acids encoding the plurality of second guide RNAs; a first RNA-guided endonuclease, or a nucleic acid encoding thereof, configured to bind to the first guide RNA; and a second RNA-guided endonuclease, or a nucleic acid encoding thereof, configured to bind to the plurality of second guide RNAs.

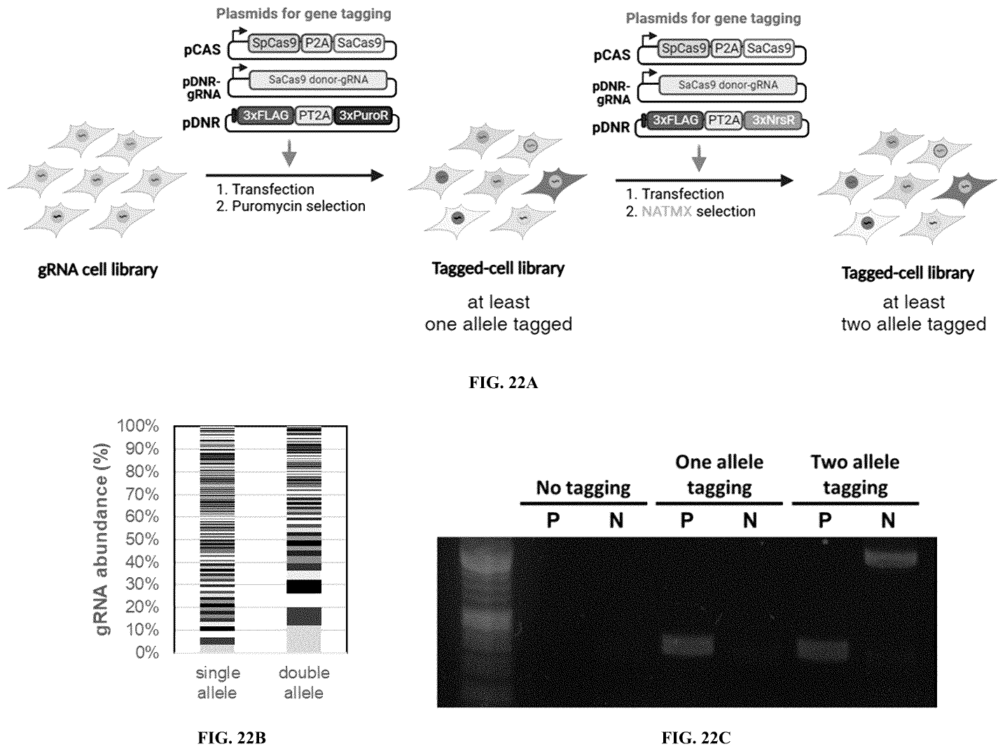

The methods herein also encompass methods comprising multiple or repeated rounds of nucleic acid modification or gene tagging. The additional rounds may utilize the same or different second gRNAs, for example to target different sequences, may utilize the same or different selectable markers, or may utilize the same or different inserts. As such, the methods may facilitate modification of both alleles, as shown in FIG. 22, potentially with different markers and/or inserts.

In some embodiments, the methods comprise contacting the plurality of target nucleic acids with a second donor nucleic acid comprising a cargo having a different selectable marker than the initial system. In some embodiments, the methods further comprise contacting the plurality of target nucleic acids with the first guide RNA, the plurality of second guide RNAs, and a first and second RNA-guided endonuclease, or one or more nucleic acids encoding thereof.

In some embodiments, the methods comprise contacting a plurality of target nucleic acids with a system disclosed herein. In some embodiments, the methods comprise contacting the plurality of target nucleic acids with a second system comprising a donor nucleic acid comprising a different selectable marker than the initial system.

The descriptions and embodiments provided above for the systems, RNA-guided endonucleases, gRNAs, and donor nucleic acid are applicable to the methods described herein.

In some embodiments, the plurality of target nucleic acids is contacted with the RNA-guided endonuclease, gRNA, and donor nucleic acid or the components of the disclosed systems simultaneously. In some embodiments, the plurality of target nucleic acids is contacted with the RNA-guided endonuclease, gRNA, and donor nucleic acid or the components of the disclosed systems at least partially sequentially.

The target nucleic acid sequence may be in a cell. In some embodiments, contacting the plurality of target nucleic acids comprises introducing, simultaneously, sequentially, or a combination thereof, the RNA-guided endonucleases, gRNAs, and donor nucleic acid, components of the disclosed systems, or a composition comprising thereof into a cell or a population of cells. For example, the plurality of second gRNAs may be introduced into a population of cells such that each cell receives a single second gRNA from the plurality of second guide RNAs. Subsequently, in any order or together the RNA-guided endonucleases, the first gRNA, and donor nucleic acid may be introduced into the population of cells or any single cell.

As described above, the RNA-guided endonucleases, gRNAs, and donor nucleic acid, components of the disclosed systems, or a composition comprising thereof may be introduced into eukaryotic or prokaryotic cells by methods known in the art. In some embodiments, the cell is a mammalian cell. In some embodiments, the cell is a human cell.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, IRNA, micro RNA (miRNA), and small interfering RNA (siRNA), and coding RNA, such as messenger RNA (mRNA). In some embodiments, the target nucleic acid sequence encodes a protein or polypeptide.

In some embodiments, the methods facilitate inserting an exogenous nucleic acid at a target site within a gene or gene product. In some embodiments, the exogenous nucleic acid or insert is inserted at the: a promoter region, an enhancer region, a repressor region, an insulator region, a silencer region, a region involved in DNA looping with the promoter region, a gene splicing region, or the N-terminal or C-terminal end of the region transcribed into the gene product, e.g., to generate an N-terminal or C-terminal fusion with the endogenous gene product. In select embodiments, the exogenous nucleic acid or insert is inserted at the N-terminus of the gene product prior to the stop codon. In select embodiments, the exogenous nucleic acid or insert is inserted at the C-terminus of the gene product after to the start codon.

In some embodiments, the methods further comprise selection of cells comprising a selectable marker, e.g., from the donor nucleic acid or from one or more of the other vectors utilized in the system. Selected cells can be colony purified and analyzed. Analysis of the transformed mammalian cells may include sequencing of the plasmids that are contained in them. The sequencing may be targeted to the segment encoding the guide RNA and the donor DNA. If a barcode is present, the sequencing may be targeted to the barcode as a surrogate for the guide RNA and the donor DNA. Any method for determining the sequence may be used. For library analysis, a massively parallel sequencing technique can be used. Typically, such techniques involve amplification before sequencing, often on a solid support, such as a bead, slide, or array. Such sequencing techniques typically involve short overlapping reads, and high coverage.

Contacting a target nucleic acid sequence may comprise administering to the subject, in vivo, or by transplantation of ex vivo treated cells. In some embodiments, the administration may be by an intramuscular, intravenous, transdermal, intranasal, oral, mucosal, or other delivery method.

The administration may be in the form of a pharmaceutical composition with a pharmaceutically acceptable carrier or excipient. In some embodiments, the RNA-guided endonuclease, gRNA, and donor nucleic acid, or components of the disclosed systems may be mixed, individually or in any combination, with a pharmaceutically acceptable carrier to form pharmaceutical compositions, which are also within the scope of the present disclosure.

In some embodiments, an effective amount of the RNA-guided endonucleases, gRNAs, and donor nucleic acid, components of the disclosed systems, or a composition comprising thereof as described herein can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the RNA-guided endonucleases, gRNAs, and donor nucleic acid, components of the disclosed systems, or a composition comprising thereof such that successful nucleic acid modification (e.g., DNA insertion) is achieved.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The disclosed methods can be used for genome-wide protein labelling, expression marking, disruption of protein expression, protein re-localization, alteration of protein expression, or high throughput screening. In accordance with these embodiments, the method would allow for both speed and precision in applications including but not limited to antibody staining of fixed cells or tissues, live imaging of protein in cells or tissues, protein capture or affinity purification for protein complex identification, cell-type lineage tracing or labeling, and production of transgenic organisms with multiple different fusions to an individual gene.

Given the methods may be completed at library scale, the methods are useful for high throughput gene modification. Accordingly, the methods are useful for high throughput genome-wide interrogation of protein function (e.g., HITAG used in combinations with single cell chromatin immunoprecipitation (ChIP) assays with sequencing, ChIP sequencing (ChIP-Seq), e.g., to map transcription factor binding at scale, and HITAG linked to degron analysis to probe regulatory networks), identification of protein localization and interaction partners (e.g., to build an interaction network, e.g., to predict genes required for disease etiology), generation large quantities of protein functions (e.g., new CRISPR effectors (e.g., activators, base editors, prime editors, inhibitors), and exploration of the effects of induced protein-protein interactions by labeling two proteins with binding partners or recruitment system components.

### Kits

Also within the scope of the present disclosure are kits that include the RNA-guided endonuclease(s), gRNA(s), donor nucleic acid, any or all of the components of the disclosed systems, or a composition comprising thereof.

The kit may include instructions for use in any of the methods described herein. The instructions can comprise a description of administration to a subject to achieve the intended effect. The kit may further comprise a device for holding or administering the RNA-guided endonuclease, gRNA, donor nucleic acid, or any or all of the components of the present system. The device may include an infusion device, an intravenous solution bag, a hypodermic needle, a vial, and/or a syringe.

The present disclosure also provides for kits for performing nucleic acid modification in vitro. Optional components of the kit include one or more of the following: buffer constituents, control plasmid, sequencing primers, culturing devices and media, and cells.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### EXAMPLES

### Materials and Methods

Plasmid construction To construct the gRNA expression plasmids, pSB700-blasto (Addgene #167904) was used for SpCas9-specific gRNA expression and a modified pSB700-vector containing a SaCas9 compatible gRNA scaffold with a zeocin-resistance gene was used for SaCas9-specific gRNA expression. Vectors containing gRNAs were cloned by Golden Gate using Esp3I. pCAS plasmids were constructed from a dual-Cas9 plasmid (Addgene #107320) by replacing the 3×HA sequence with a P2A sequence using Gibson assembly. pDNR was constructed from pCRISPaint-TagGFP2-PuroR (Addgene #80970). TagGFP2 was replaced with mCherry using BamHI/ZraI double digestion. To construct the modified P3 donor with additional copies of the puromycin resistance gene two puromycin resistance genes were PCR amplified with primers designed to add a T2A sequence to their end each coded using a different set of synonymous codons. These fragments were then assembled into a version of pDNR that was digested with ZraI using gibson assembly. All plasmids were validated by Sanger sequencing and will be made available via Addgene.

Target-gRNA design To design target-gRNAs the CRISPick tool from the Broad was used with settings Human GRCh38, CRISPRko, and SpyoCas9. Guide RNAs with Esp3I restriction sites inside of them, a polyT stretch longer than 4 base pairs, or more than one exact match in the huma genome were excluded from use even if they were the gRNA closest to the stop codon. Frame number was categorized as the number of bases required to complete the cut codon after cleavage.

Construction of HEK293T cell lines with on average a single target-gRNA HEK293T cells were cultured in Dulbecco's Modified Eagle Medium (DMEM)+10% FBS+1% penicillin/streptomycin and incubated at 37° C. and 5.0% CO₂. On day 1, HEK293T cells were seeded ˜3.5×10⁶ cells per well on 6-well plates for lentivirus production. On day 2, a mixture of 600 ng psPAX2, 150 ng pMD2.G, and 450 ng of the target-gRNA plasmids were transfected using lipofectamine 2000. A mixture of 125 μl OPTI-MEM and 5 μl lipofectamine 2000 was incubated for 5 minutes and added to the plasmid mixture. The DNA-lipofectamine complex was formed by incubating 20-30 minutes and then slowly dribbled onto each well. On day 3, the media was changed. Lentivirus was harvested by collecting supernatant after centrifugation (500 g for 5 minutes) of the media on day 4 and day 5. The lentivirus stocks were stored at −80° C. in 1 ml aliquots. To generate HEK293T cells with on average a single target-gRNA integrated into their genome viral stocks were tested for infectivity and cells were infected at an MOI of ˜0.1.

Quantifying the efficiency of tagging When optimizing the tagging approach, cells were plated at a density of 5-7×10⁴ cells per well in a 24-well plate with a media volume of 600 ul. Plasmids for transfection were prepared by mixing 100 ng pCAS, 100 ng pDNR-gRNA, and 200 ng pDNR or as described in the manuscript. When confluency reached 50-70%, plasmids were transfected using lipofectamine 2000, according to the manufacturer's protocol. Media was changed on the next day of transfection.

On day 6 after transfection, the transfected population was split to PDL-treated 96 well plates at a density of 10⁴-2×10⁴ cells per well. The following day cells were fixed with 4% paraformaldehyde for 5 minutes. The cells were washed with PBS, stained with DAPI for 5 minutes, and washed twice again with PBS. The cells were imaged using ImageXpress® Pico Automated Cell Imaging Systems. Tagging efficiency was determined by the number of mCherry-positive cells showing the proper localization over the total number of cells as determined by DAPI staining.

Creating HITAG libraries When performing HITAG cells were transfected as above and split into non-drug media until day 7 when they were split into PDL-treated 24 well plates at a ratio of 1:8 and grown in media containing 0.5 ug/ml puromycin. The media was then changed every 2-3 days. The cells were grown for 1-2 weeks until non-transfected cells died out. To expand the tagged lines cells were detached and resuspended in fresh media without puromycin.

To initiate tagging gRNA cell libraries, 6-well plates the cells were seeded with 2.5-5.0×10⁵ cells per 6-well. One 6-well plate were used per library. On the next day, HITAG plasmids were transfected with 5-times more plasmid and transfection reagents used for 24-well plate transfection. Cells were split 1:4 in media with 0.5 ug/ml puromycin 7 days after the transfection. The selection stopped until the next day when the negative control cells died out. Selected tagged cells were expanded by changing media.

A stress granule library of HCT116 cells was generated by going through the same procedure except the concentration of drug (blastidicin, 5.0 ug/ml; puromycin, 2.5 ug/ml), transfection reagent (FugeneHD), and the number of transfected cells (4 T75 flasks).

Construction of stress granule library A list of stress granule associated genes was collected from the existing literature. These methods identified SG proteins based on biotinylation of proteins in close spatial proximity to core SG components or affinity purification of SG followed by mass spectrometry. Using this list, a set of target-gRNAs against each gene was generated with any target-gRNAs that would result in a loss of more than 6 amino acid from the C-terminus of a protein being removed. The resultant list of target-gRNAs was then sorted into three libraries based on the frame.

Three gRNA libraries with different frames were synthesized as oligo pools from Agilent. Each gRNA library was then PCR amplified from the initial oligo pool and cloned into the modified pSB700 vector using Golden Gate assembly.

gRNA analysis 1-2×10⁶ cells were harvested and washed once with PBS. The cell pellet was resuspended in 500 ul Lucigen DNA QuickExtract reagent and incubated at 65° C. with shaking at 750 rpm for 15 minutes. After brief centrifugation, the sample was incubated at 95° C. with shaking at 750 rpm for 5 minutes. gRNA regions were PCR amplified using the following conditions: 98° C. 45 s, [98° C. 15 s; 56° C. 15 s; 72° C. 20 s]×n, 72° C. 2 min, 4° C. hold, where n is the cycle number which was determined empirically to be before the PCR reaction saturated (usually between 20-25 cycles). The second round PCR was performed to add the Illumina indices: 98° C. 45 s, [98° C. 15 s; 56° C. 15 s; 72° C. 20 s]×8, 72° C. 2 min, hold at 4° C. The PCR products were then run on a 2% agarose gel and the band of interest was purified. Samples were then sequenced on an Illumina NextSeq 500. The resulting reads were then analyzed by either aligning them using Bowtie2 or using MAGeCK to process the resulting reads.

Isolation and analysis of the target-mCherry junction Genomic DNA was extracted from 1-2×10⁶ cells using the Qiagen DNA extraction kit (#69504). Enzymatic DNA fragmentation was performed using the NEBNext® Ultra™ II FS DNA Library Prep Kit (E7805S). 2 ug of genomic DNA (500 ng per reaction) was treated with 5 minutes of enzymatic fragmentation. All subsequent steps were performed as instructed by the manufacturer. The DNA fragments containing the mCherry sequence was amplified through a nested PCR approach. For the first round PCR, 500 ng of fragmented and adapter ligated DNA (50 ng per reaction) was amplified using a primer binding on the reverse strand of the mCherry sequence and another primer binding to p7 adaptor under the following PCR condition: 98° C. 45 s, [98° C. 15 s, 65° C. 15 s, 72° C. 90 s]×20, 72° C. 5 min, hold 4° C. After the resulting sample was purified using SPRI beads aiming to capture all products greater than 100 bp in size. The second-round PCR reaction was then performed using 50 ng of the first-round PCR product (10 ng per reaction) and primers under the following PCR condition: 98° C. 45 s, [98° C. 15 s, 65° C. 15 s, 72° C. 90 s]×8, 72° C. 5 min, hold 4° C. The final PCR products were then isolated using SPRI beads aiming to remove all products smaller than 200 base pairs.

To characterize the target gene mCherry tag junctions, a database of all genomic regions adjacent to the target cut sites was constructed. NGS reads were then blasted locally twice, once against the database of target genomic regions, and once against the three linker sequences, which differ by a few bases in order to maintain the appropriate reading frame. Reads which featured at least 20 bases of alignment to genomic targets and at least 20 bases of alignment to linker sequences were analyzed by comparing the locations of the alignments to the expected location based on the gRNA cut site. Insertions were identified as sections of a read which did not align to either the genomic target or the linker sequence, while deletions appeared as a difference between the expected cut site and the observed cut site.

Generation of clonal cell lines and identification of the gRNA in each well. The library of cells with mCherry tagged SG-associated proteins were detached from a T75 flask and washed once with prechilled PBS. The cells were resuspended in Ca/Mg-free PBS+1% FBS, filtered with a 50 um mesh filter and kept in ice. Before single-cell sorting, SYTOX Blue (1:1000, Thermo S34857) was added as a cell viability indicator. In preparation for single cell sorting, 96-well plates were filled with 150 ul of media and prewarmed to room temperature. Viable cells were sorted on the 96-well plates using Sony MA900 in the Single-Cell Mode. The media from all plates was then changed as needed. Each well was confirmed visually to have one colony per well after 10 days.

PCR-ready genomic DNA was prepared by mixing ˜2×10⁴ cells in each well with 30 ul Lucigen DNA QuickExtract. After incubating the plates for 15 minutes at 65° C. followed by 10 minutes at 95° C., 1 ul of the DNA extract was used for PCR to amplify the gRNA sequences. The same PCR condition was used as described above except 35 cycles was used during round 1. Read counts of gRNAs for each well were then analyzed. For a gRNA to be identified in a given well it needed to be present at an abundance at least 3 times greater than the next most abundant gRNA in that same well.

Immunofluorescence staining for stress granule formation. Sodium arsenite stress was applied by incubating cells with media containing 0.5 mM NaAs₂O₃ for an hour. Cells were then washed once with PBS and immediately fixed with 4% paraformaldehyde for 5 minutes, washed twice with PBS+0.1% TritonX-100 incubating for 10 minutes between each wash step, and blocked with Superblock™ Blocking Buffer (Thermofisher #37581) with 0.1% TritonX-100 for 2 hours at room temperature. For primary antibody staining, cells were covered with 100 ul Superblock with 0.1% TritonX-100 and primary antibodies (G3BP1, proteintech 13057-2-AP, 1:1000 dilution; mCherry, proteintech 68088-1, 1:1000 dilution) overnight at 4° C. After washing the cells with PBS+0.1% TritonX-100 (15 minutes), Superblock with 0.1% TritonX-100 (15 minutes) cells were incubated with secondary antibodies (Goat anti-mouse, Invitrogen A32727; Goat anti-rabbit, Invitrogen A32731, 1:1000 dilution) for an hour at room temperature. After one wash with PBS+0.1% TritonX-100, cells were stained with 0.1 ug/ml DAPI in PBS for 5 minutes followed by two PBS washes. At this point plates were either immediately imaged or covered with aluminum seals and stored at 4° C.

Collection of protein features Three published algorithms including PSPredictor, CatGranule and Plaac were used to predict LLPS score. Numbers of intrinsically disordered regions, number, and fraction of charged residues, hydropathy, and net charge were calculated using CIDER. All the scores were predicted with default parameters using the natural protein sequences.

Protein-protein interaction network analysis The protein-protein interaction network was extracted from the STRING database, with network type as physical network and a minimum required interaction score as 0.400. All of the text mining, experiments, and databases were accepted as active interaction sources. Orphan genes (the gene whose degree is 0) are not included in the final network. K-means was used for clustering, and the cluster number was set to 2. Visualization is made by Gephi 0.9.2, with different colors indicating different gene groups and node size indicating node degree.

### Example 1

### Generating Pools of Tagged Cells Using CRISPR+NHEJ

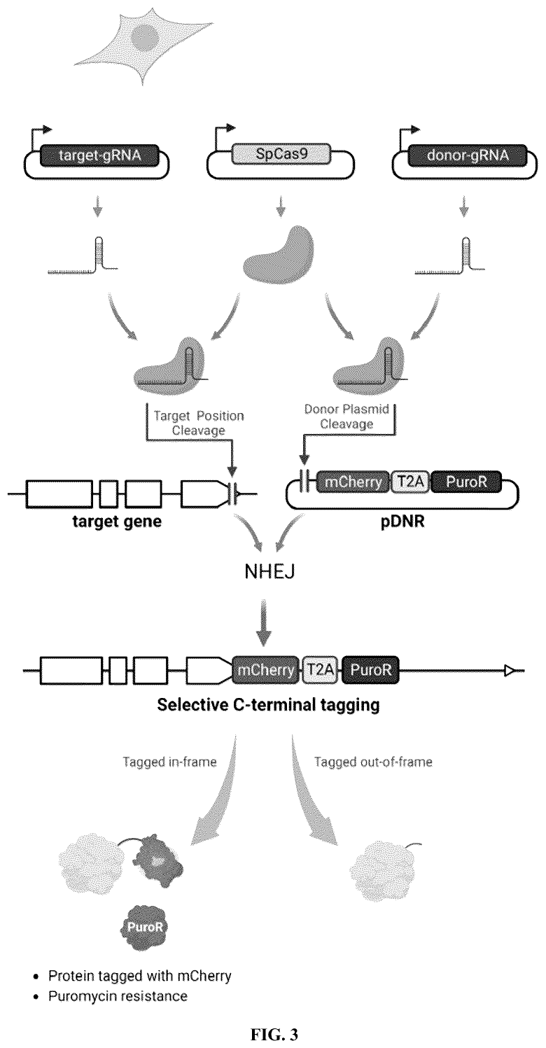

In a previous method of NHEJ-based endogenous gene tagging termed CRISPaint, a donor plasmid containing the tag to be inserted into the genome is transfected into cells. Along with the donor plasmid, 3 other plasmids containing Cas9, a gRNA against the C-terminus of the gene to be tagged (target-gRNA), and a gRNA against the donor plasmid (donor-gRNA) are also delivered. Once all plasmids are inside the cell, the target-gRNA and donor-gRNA complex with Cas9 and cut the target gene and the donor plasmid, respectively. The cleaved plasmid can then become ligated into the endogenous locus via NHEJ. If the tag gets knocked in-frame to the gene of interest it will also lead to the expression of a drug resistance marker, enabling the facile enrichment of properly tagged cells by applying drug selection to the pool of transfected cells (FIG. 3).

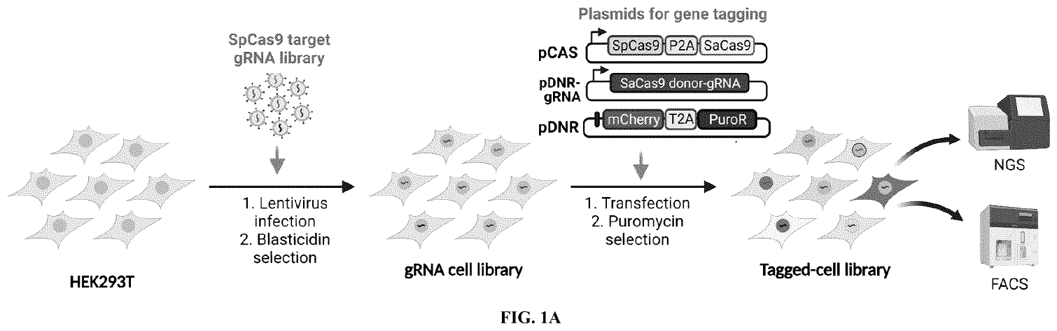

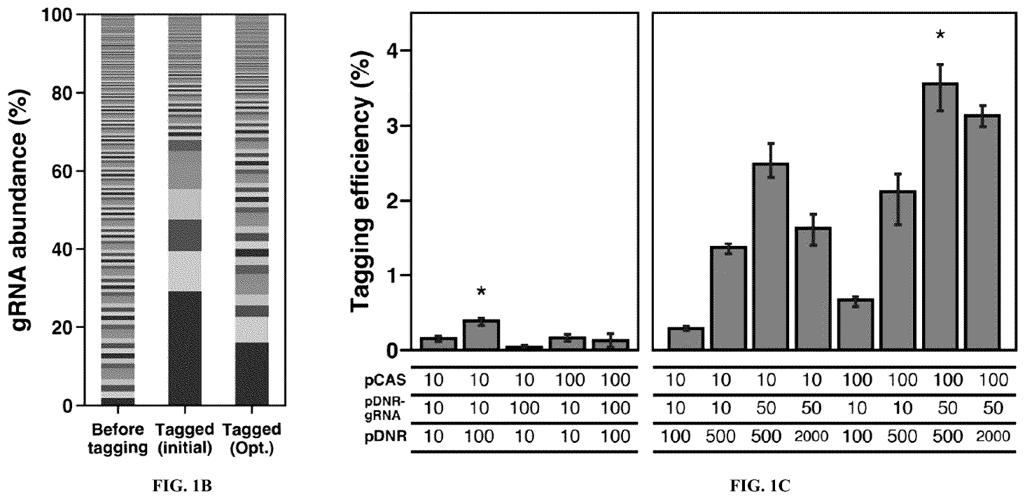

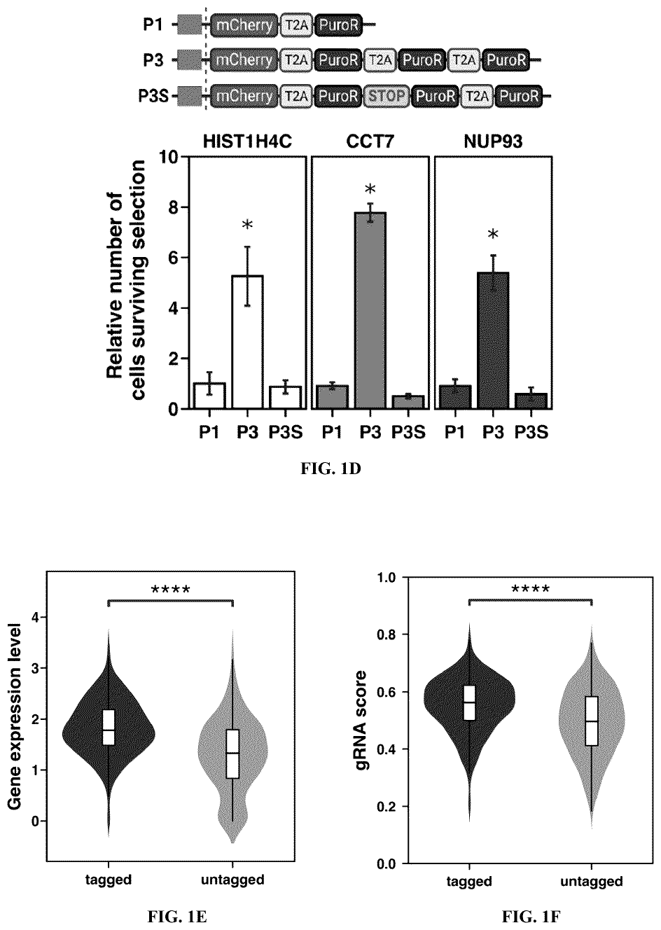

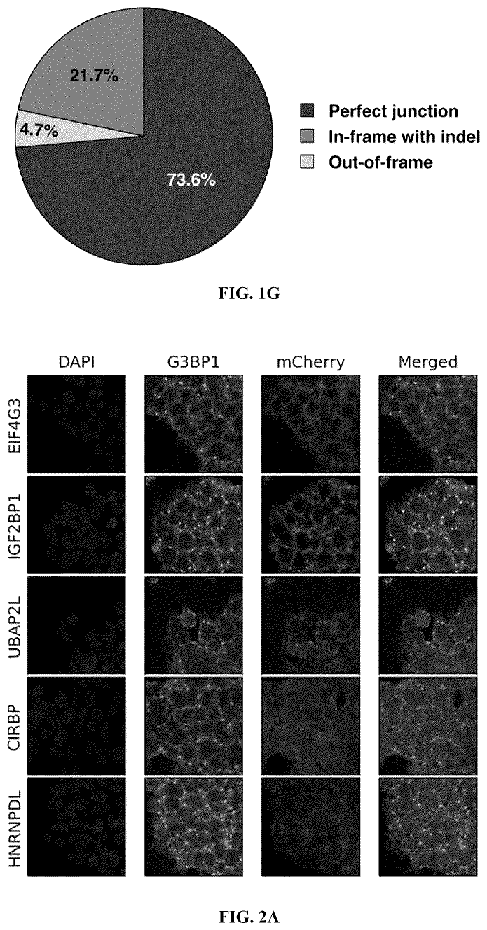

To adapt this system into one suitable for rapidly generating libraries of tagged lines, the necessity for performing independent transfections for each gene to be targeted needed to be removed. To address this bottleneck a mixture of target-gRNAs was packaged into lentiviruses and delivered to cells at a low multiplicity of infection (MOI˜0.1) to ensure that on average each cell integrates a single target-gRNA into its genome. This pool of cells can then be transfected en masse with the remaining components required for tagging (Cas9, donor plasmid, donor-gRNA), thereby enabling each cell to uniquely tag the gene to which its integrated target-gRNA is directed against (FIG. 1A). Finally, to enrich for properly targeted cells, a round of drug selection is applied.

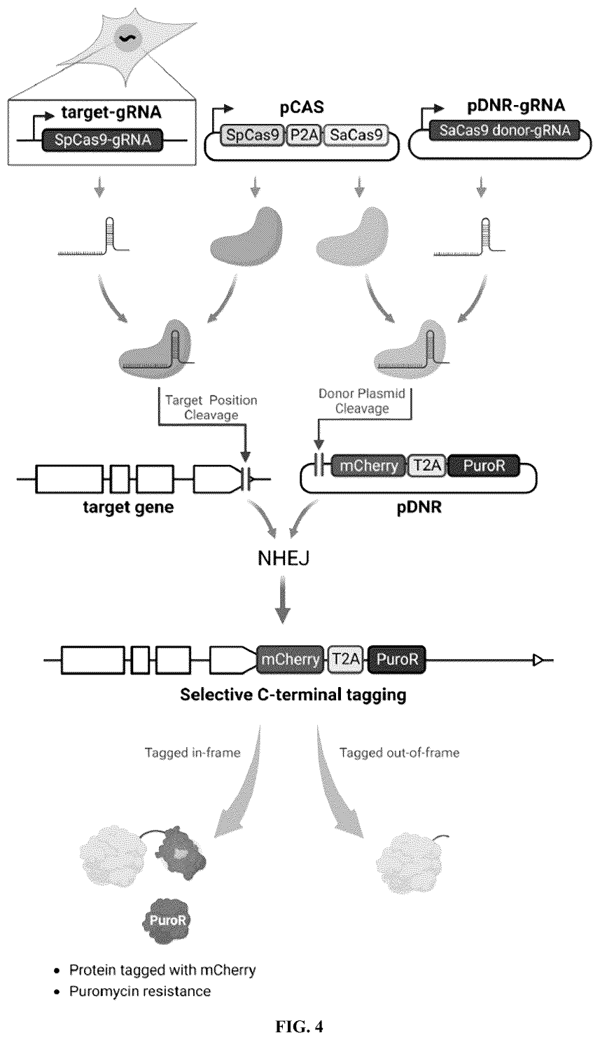

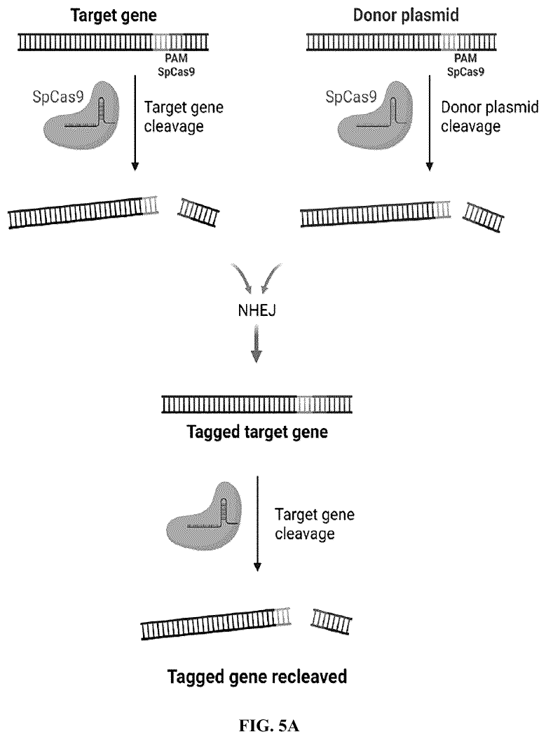

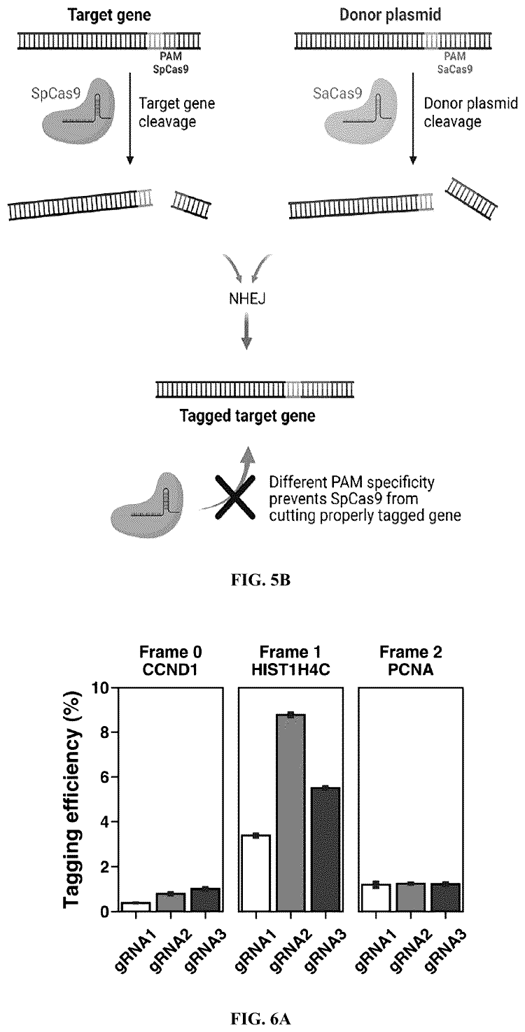

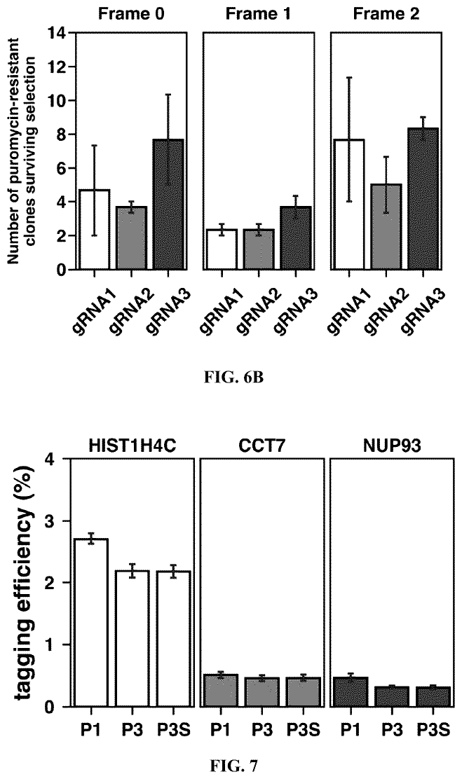

To avoid competition between the target-gRNA and donor-gRNA for complexing with Cas9, since if competition did occur it would interfere with either cleavage of the endogenous locus or the donor plasmid and in doing so decrease the rate of knock-in, two orthogonal Cas9 proteins, SpCas9 and SaCas9, were employed, each of which has a unique gRNA scaffold it interacts with which is orthogonal to the other. In this design, SpCas9 is used to cleave the endogenous target gene and SaCas9 is directed to linearize the donor plasmid (FIG. 4). Another benefit of using two orthogonal Cas9 proteins is that properly tagged genes will not be recut, which can occur when only using a single Cas9 protein should the target gene and the donor plasmid have a similar sequence proximal to their PAM sites (FIGS. 5A-5B). Finally, to reduce off-target insertions which can occur if the donor-gRNA were to inappropriately cleave the genome, several different SaCas9 gRNAs were examined. From this analysis a gRNA that showed limited off-target activity and high tagging efficiency was identified and used throughout the remainder of the study (FIGS. 6A-6B).

Stress granules (SG) are transient liquid-liquid phase separated (LLPS) RNA-protein complexes that form in response to environmental stress. To date, the factors which drive certain proteins to accumulate strongly within SG remains unclear. By tagging a large number of SG-associated proteins with the fluorescent protein, mCherry, insight could be gained into the properties that drive strong versus weak accumulation in SGs. Having established the initial approach to high-throughput gene tagging, a 193-member target-gRNA library against SG-associated proteins was designed and delivered to HEK293T cells (FIG. 1a). Into the resulting mixture of cells, the remaining plasmids required for tagging were transfected in and puromycin-resistant cells were then selected. To determine the bias in tagging in the resulting library, the abundance of each gRNA within the population of cells before and after tagging was determined. The distribution of gRNAs in the initial population of cells before mCherry knock-in was even, with no gRNA dominating the pool (FIG. 1B). In contrast, after mCherry insertion and puromycin selection a marked skew in the population was observed with 5 gRNAs occupying more than 60% of the pool, suggesting that only a small handful of genes were efficiently tagged during the initial attempt.

### Example 2

### Optimizing Insertion Rates and Drug Selection Steps

Using a HEK293T cell line that stably expressed a single target-gRNA against the histone gene, HIST1H4C, 10 ng or 100 ng of each of the plasmids required for tagging (Cas9, donor plasmid, donor-gRNA) were delivered (FIG. 1C). These studies revealed that a higher amount of the donor plasmid (100 ng) and a lower amount of the donor-gRNA (10 ng) led to a significant increase in the number of mCherry positive cells, before the application of drug selection. Further development of the transfection conditions demonstrated an upper limit to the benefit of delivering more donor plasmid (500 ng), improvements by adding additional Cas9 plasmid (100 ng), and a decrease in insertion rates when the amount of donor-gRNA is further reduced relative to the amount of donor plasmid (FIG. 1C). These results indicate that the amount of cleaved donor plasmid is a factor in determining the tagging efficiency and that cutting the donor plasmid too early, via increased amounts of donor-gRNA, before the endogenous locus is cleaved may be detrimental.

To increase the level of puromycin resistance conferred upon tagging, a modified donor plasmid (P3) was constructed containing three tandem copies of the puromycin resistance gene downstream of the mCherry tag (FIG. 1D). As a control, a donor plasmid with three copies of the puromycin resistance gene downstream of mCherry, but containing a stop codon after the first PuroR copy was also constructed (P3S). These modified donor plasmids along with the original donor plasmid with one copy of the PuroR gene (P1) were then transfected along with Cas9 and the donor-gRNA into HEK293T cells containing a single target-gRNA against either HIST1H4C, the chaperonin complex member, CCT7, or the nuclear pore protein, NUP93. Minimal difference in the number of mCherry positive cells before puromycin selection was observed among the different donors (FIG. 7). In contrast, the number of viable cells dramatically increased after puromycin selection using the P3 donor plasmid which expresses the three copies of the puromycin resistance gene (FIG. 1D). These results suggest that in the initial approach many properly tagged cells were being lost due to insufficient expression of the drug selection marker.

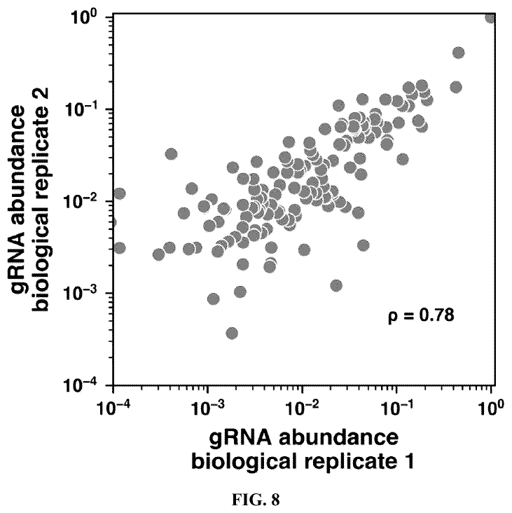

Upon applying the improved transfection conditions and optimized P3 donor plasmid design to the initial 193-member SG target-gRNA library, a marked improvement in the number of tagged genes was observed. Of the genes that were tagged in the pool 29 genes showed an abundance>1% in the pool as compared to only 8 genes meeting this threshold in initial approach (FIG. 1B). In addition, a strong correlation (p=0.78) among independent biological replicates was also found (FIG. 8). This optimized pipeline for generating pools of tagged cells is referred to as High-throughput Insertion of Tags Across the Genome or HITAG.

### Example 3

### Application of HITAG to Stress Granule Factors

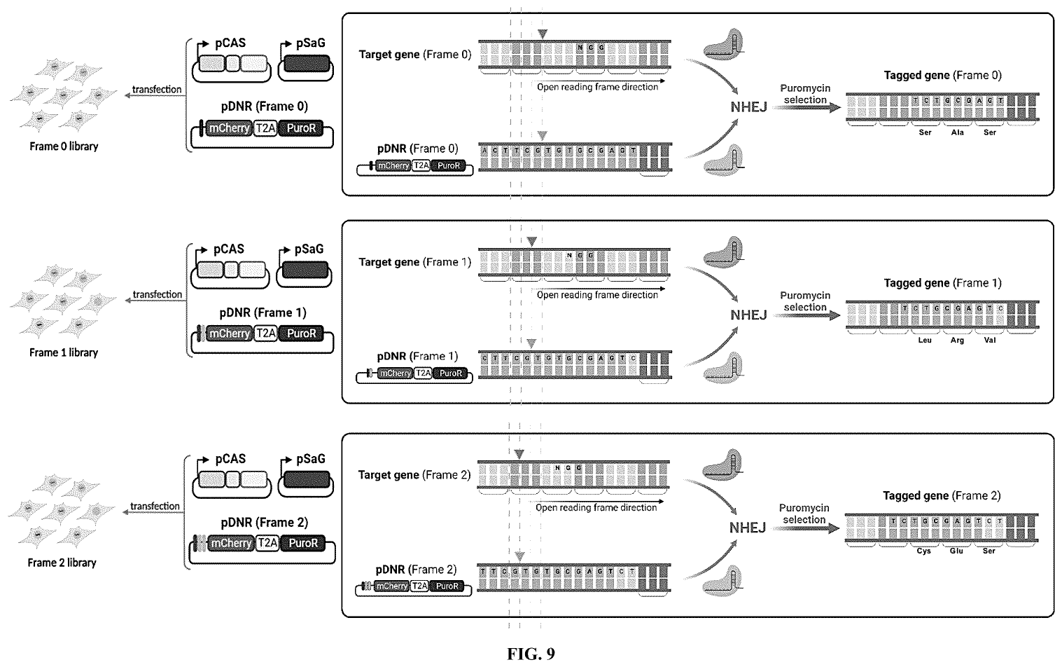

In HITAG, a target-gRNA is designed to cut upstream of the stop codon such that the fused tag is translated with the target gene. Upon cutting the C-terminus of a target gene there are three possible reading frames to which a donor vector can be fused, with only one leading to an in-frame translated tag. Previous studies have shown that to increase tagging efficiency genes that produce the same reading frame when cut should be grouped together (FIG. 9). The initial 193 SG targets all shared the same reading frame upon being cut by Cas9. To tag the remaining proteins previously associated with SGs two additional target-gRNA libraries, 190 and 205 members in size, were created and used to generate additional pools of mCherry tagged cells via the HITAG approach.

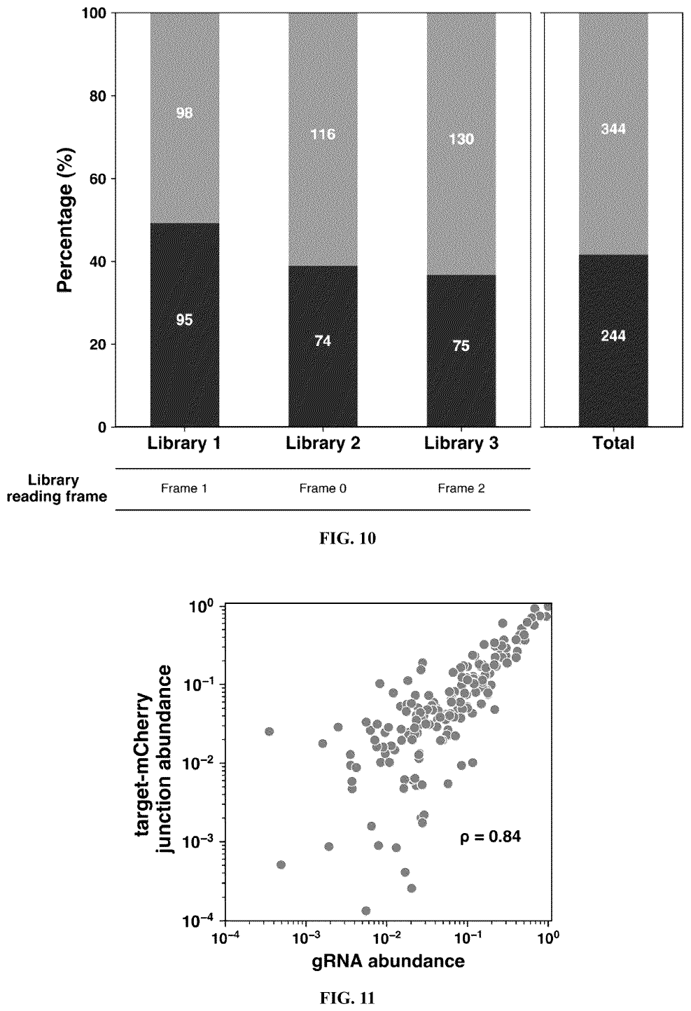

To begin to characterize the fidelity of HITAG, the junctions between the various target genes and the inserted mCherry tag (junction reads) were selectively enriched via a nested PCR approach. From these analyses 244 of the 588 genes that were targeted across the three reading frame libraries were tagged and survived drug selection (FIG. 10). The number of junction reads associated with each tagged gene were then compared to its respective target-gRNA abundance within the same pool of cells. Good correlation between these two metrics was observed (p=0.84), suggesting that the target-gRNA within each cell is driving the tagging events and that target-gRNA abundance is a reasonable surrogate for which genes are tagged within each cell (FIG. 11).

Comparing the expression between genes that were tagged to those that were not, showed a significant difference between these two sets of genes, with tagged genes showing on average higher levels of expression (FIG. 1E). While expression is important, several genes showed high expression but remain poorly tagged. In order to reduce the number of amino acids lost from the C-terminus of the various target genes upon tagging, gRNAs were selected based on their proximity to the stop codon. It was hypothesized that a portion of the variation in tagging may be driven by inefficient target-gRNAs that poorly cut the endogenous target site. When analyzing the on-target efficacy score of various target-gRNAs, tagged genes were associated with target-gRNAs that had higher predicted efficiency scores as determined by CRISPick (FIG. 1F).

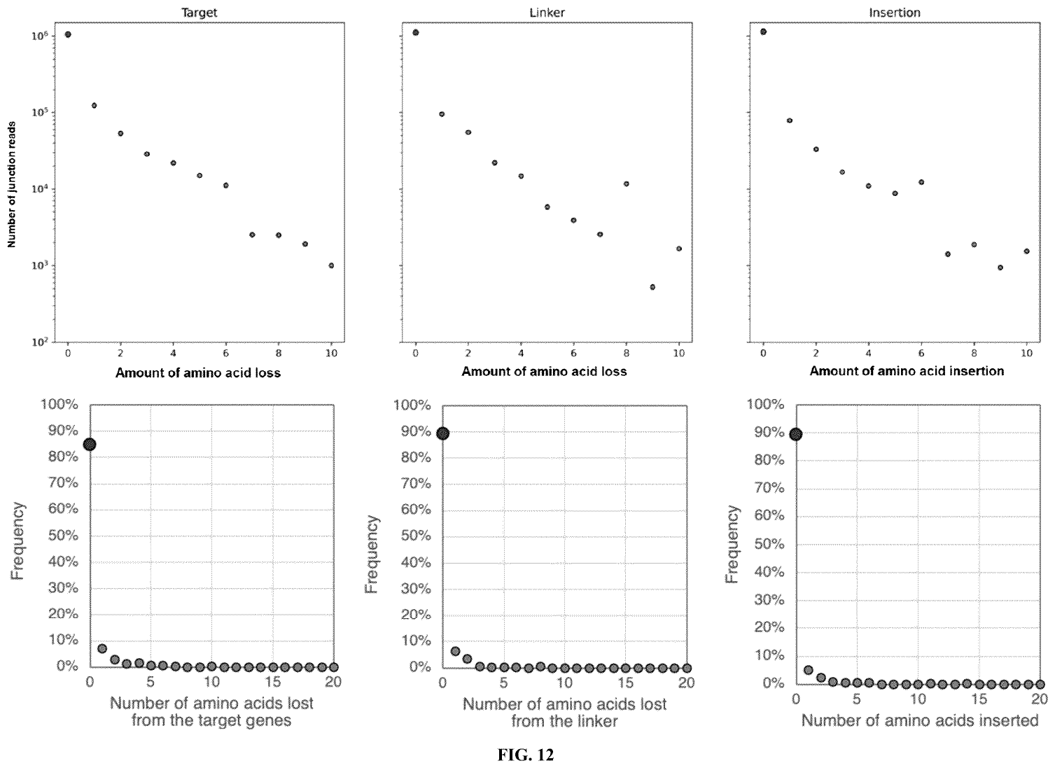

When analyzing the sequenced junctions from the pool of mCherry tagged cells, 72.7% of all junctions were precise fusions between the endogenous locus and the donor plasmid (FIG. 1G). Of the remaining junctions 22.6% retained the proper frame between the gene of interest and the mCherry tag but showed a loss or gain of bases, with a strong bias towards smaller 1-2 amino acid deletions and insertions within the observed repair products (FIGS. 1G and 12). Of the remaining repair junctions 4.7% were out-of-frame products, presumed to arise from cells that have both a properly tagged allele conferring drug resistance and an out-of-frame allele.

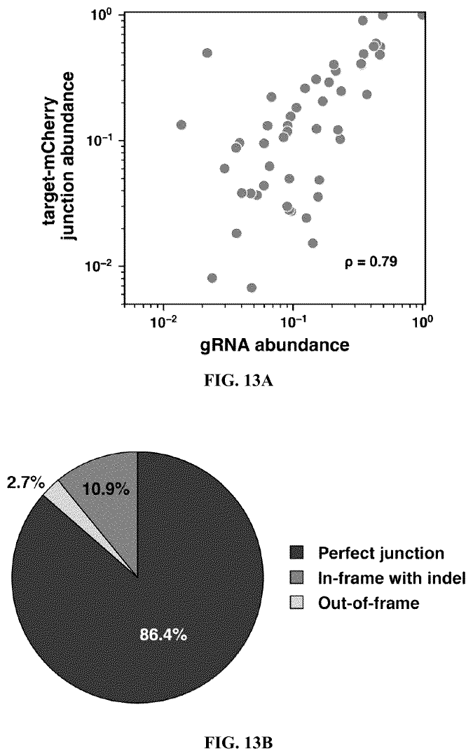

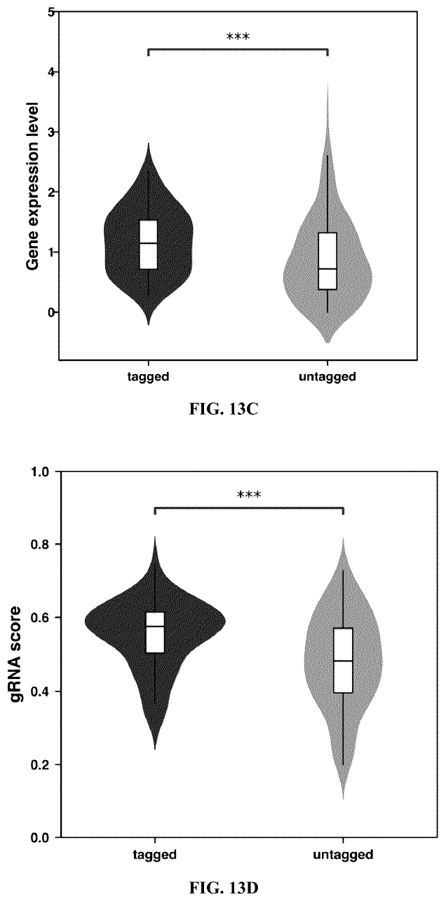

To investigate whether the HITAG approach can be applied to other cell lines, a set of 205 stress granule associated genes were tagged with mCherry using the human colorectal carcinoma cell line, HCT116 (FIGS. 13A-13D). The features of tagging such as the efficiency, specificity, overall repair profile, and biases in tagging were similar between HCT116 and HEK293T cells, suggesting that HITAG represents a general method for tagging genes in high-throughput.

### Example 4

Characterization of Tagged Lines and their Association with Stress Granules

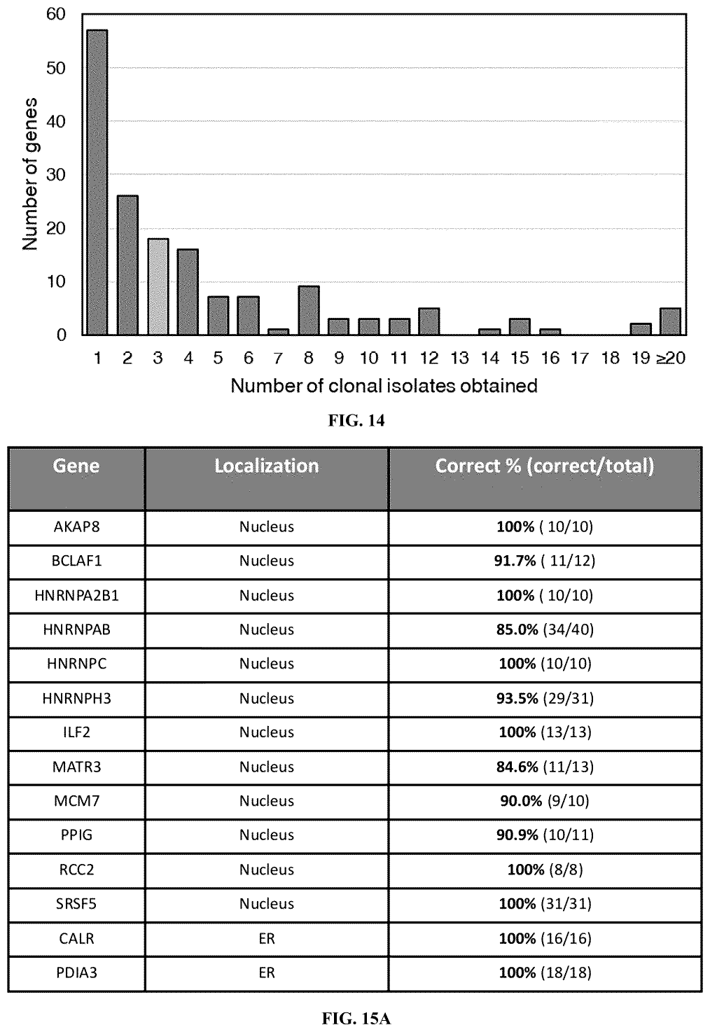

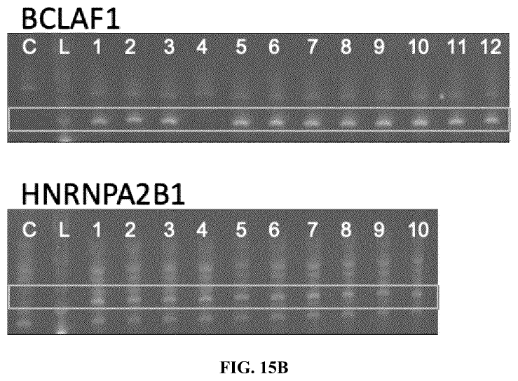

To examine the behavior of the tagged proteins within the mixed pool, single cells were isolated, grown clonally, and the gRNA inside each was sequenced. Within the 806 clonal lines obtained, 167 unique proteins were mCherry tagged, with each protein being represented by a median of 3 clonal isolates (FIG. 14). To quantify the rates of false targeting, proteins that were tagged within at least 10 independent isolates and showed distinct subcellular localization were examined (FIGS. 15A-15B). On average ˜95% of the clones for a given protein showed the expected pattern of mCherry localization, with results for two targets, BCALF1 and HNRNPAB, also being confirmed by PCR.

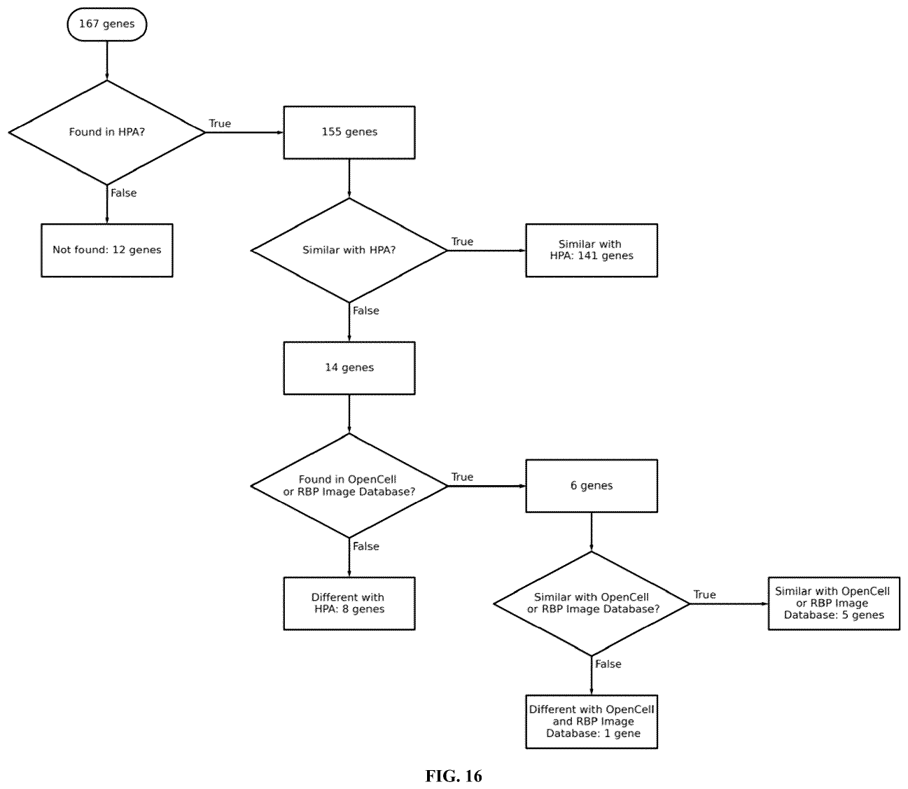

To probe if the mCherry label alters protein localization, the cellular distribution of the tagged proteins (FIG. 16) was compared with the annotations listed within the Human Protein Atlas. Of the 155 proteins with localization data in the Human Protein Atlas, 141 agreed with the findings. For the 14 proteins with conflicting results, 6 had annotations in other databases, with 5 of these agreeing with our findings. As a whole these data suggested that HITAG generates a high percentage of cells where the label is inserted as designed and that the C-terminal fusion of mCherry to proteins of interest rarely perturbs their localization.

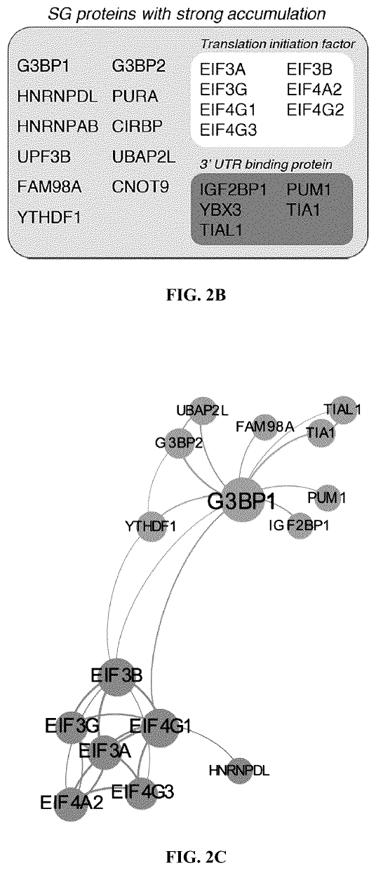

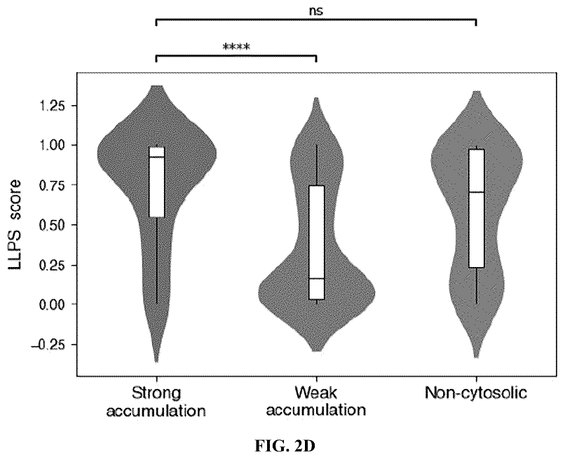

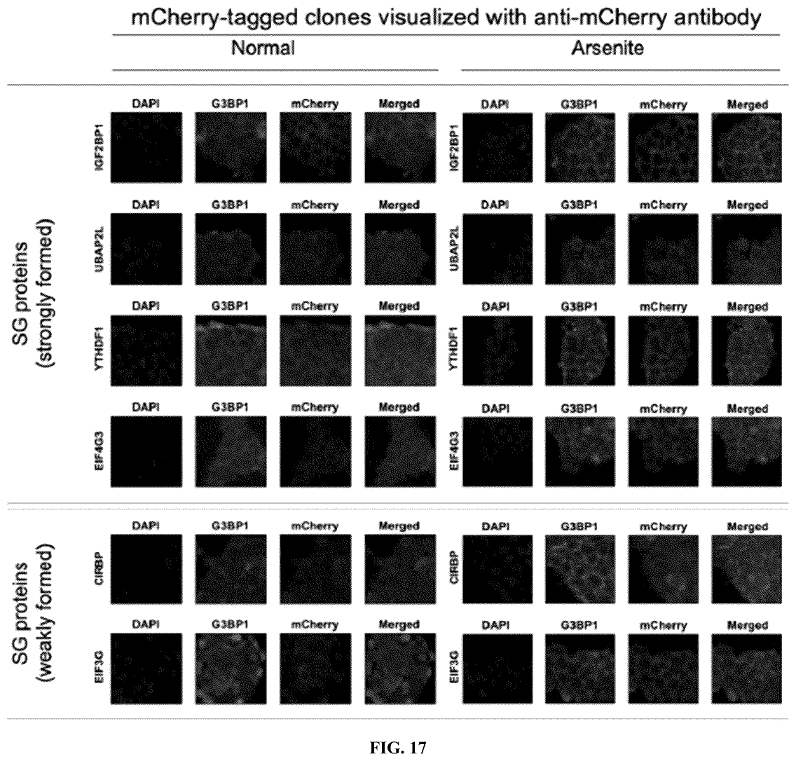

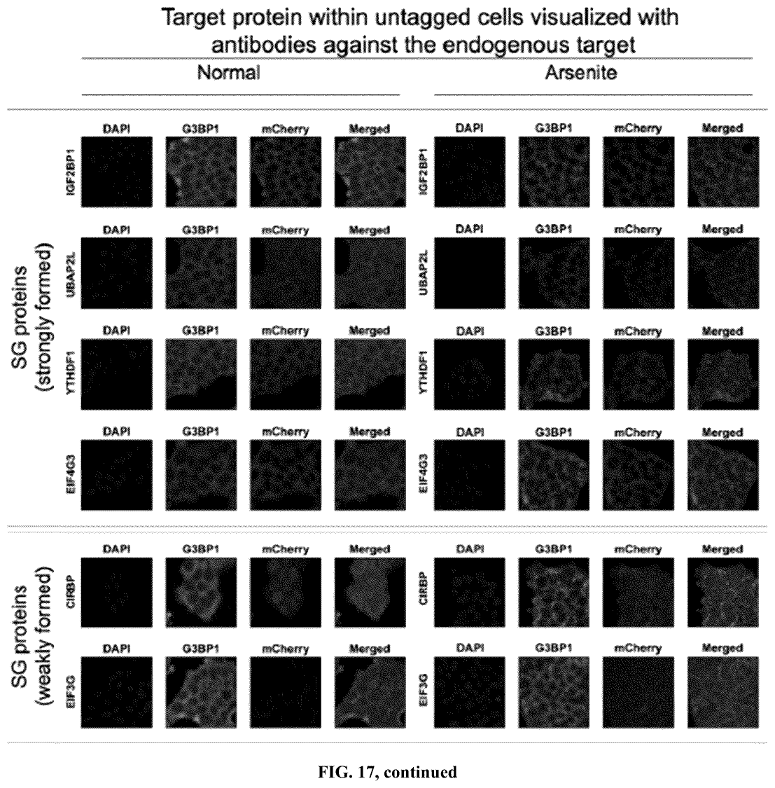

While there are hundreds of proteins that have been found in SGs, what drives their accumulation and why some proteins are more efficiently recruited to SGs is under active investigation. To probe these questions, all 167 clonal lines were treated with 0.5 mM sodium arsenite for 1 hour to induce SG formation, which was visualized by staining for the canonical SG marker G3BP1 (FIG. 2A). Of the 167 tagged proteins only 23 showed strong accumulation within stress granules, as determined by fluorescence microscopy (FIG. 2B). To determine if the mCherry label might be affecting protein dynamics, 6 proteins that showed either strong or weak accumulation within SGs were stained with antibodies against the target protein in wild-type cells and the results compared to the findings (FIG. 17). Comparisons between the staining patterns showed that the mCherry tag had a minimal effect on altering a given protein's interaction with SGs.

In examining the 23 proteins with strong accumulation several features immediately became apparent. Among the hits, all showed predominantly cytoplasmic localization and had associated RNA binding activity. In addition, the majority could be clustered by protein-protein interactions into two groups centered around either EIF4G1 or G3BP1 (FIG. 2C). These observations are consistent with the fact that SGs form during times of translation inhibition in which stalled preinitiation complexes, containing EIF4G1, and their associated RNAs undergo condensation upon interaction with key nucleating RBPs such as G3BP1.

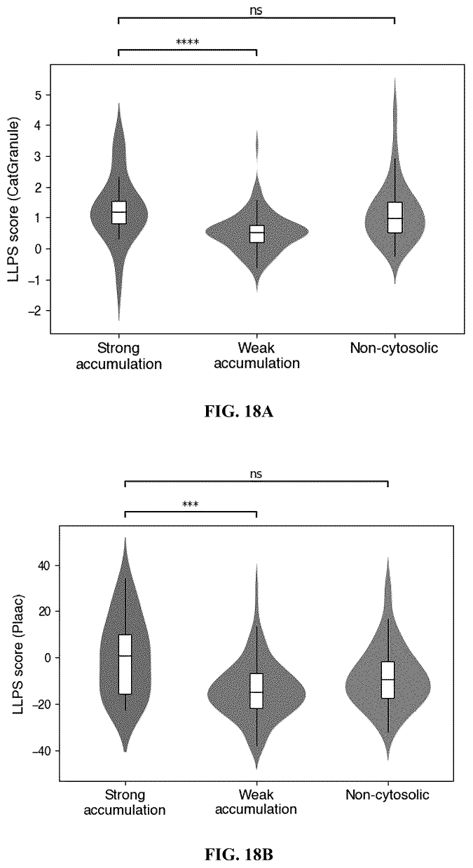

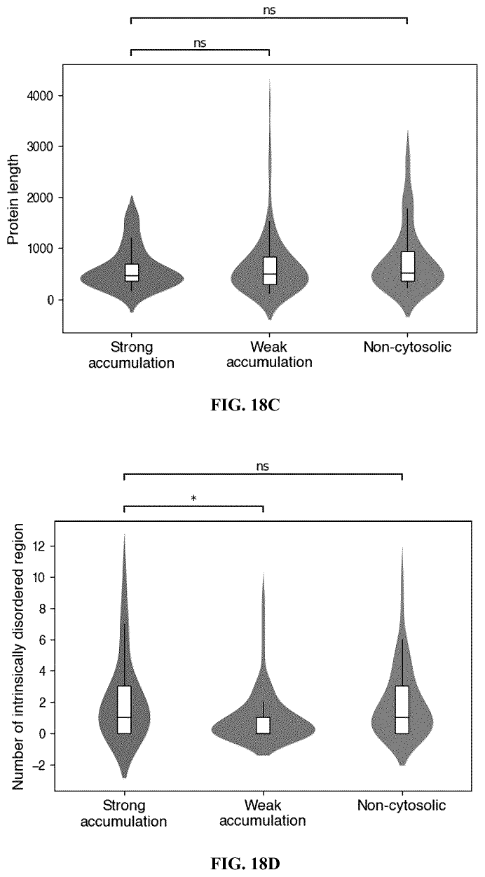

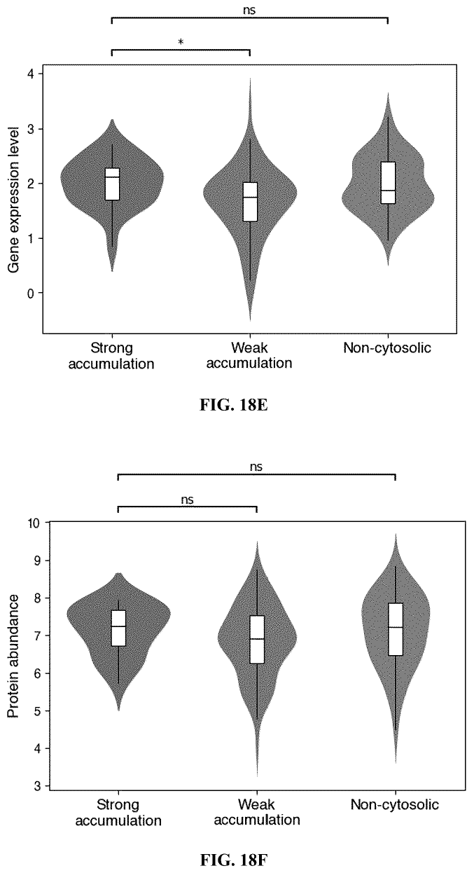

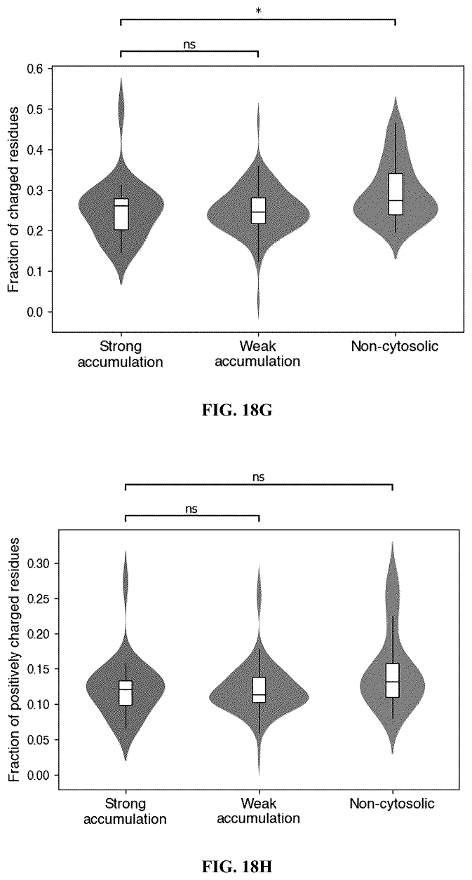

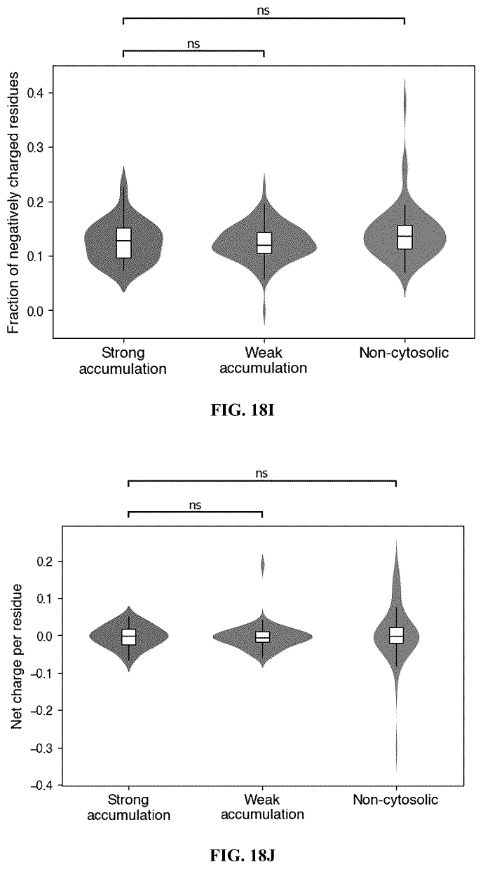

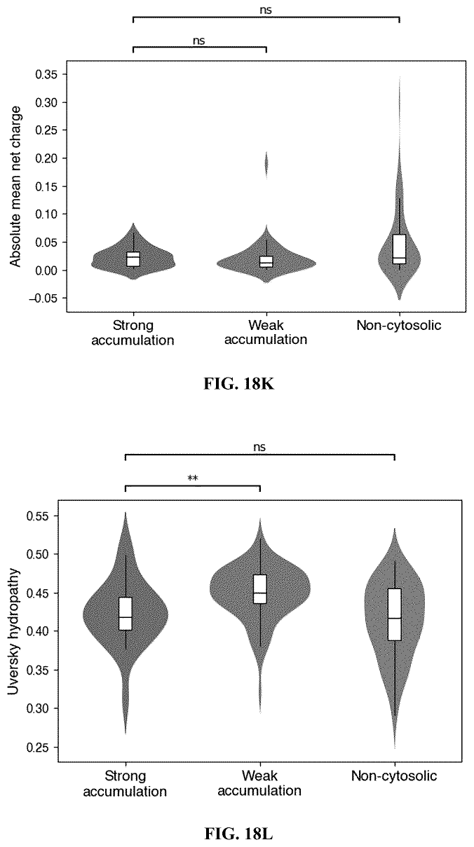

To further characterize the nature of proteins that show strong association with SGs hits were scored for a variety of features such as protein length, size of intrinsically disordered regions, abundance, and charge (FIGS. 2D and 18A-18L). Somewhat surprising features such as protein length or number of protein interaction partners showed no correlation with the strength of accumulation within SGs. Of the metrics examined, a propensity to liquid-liquid phase separate (LLPS) showed the greatest difference between strong and weak accumulators. This feature of LLPS suggests that intermolecular interactions are critical to the strong recruitment and retention of particular proteins within the SG. It also is in line with data showing that a combination of protein-protein and protein-RNA interactions are essential to drive SG formation. As a whole the findings suggest that strong recruitment to SG requires a protein to have a predominantly cytoplasmic localization, ability to interact with RNA, and an ability to phase separate. In line with these conclusions, several nuclear RNA-binding proteins were found to have high LLPS scores but poor accumulation within SG within our dataset. Yet, previous reports have shown that during times of stress some of these proteins can become localized to the cytoplasm which then enables them to robustly accumulate within SGs.

The coupling of HITAG with single-cell approaches such as pooled optical screens finds use in the isolation of clonal populations post tagging and provide a further boost to discovery throughput. To overcome the fact that lowly expressed genes are being lost due to insufficient levels of drug marker expression additional copies of the puromycin resistance marker, beyond the three currently being used, may be used. Alternatively, more efficient drug markers may be used. In some embodiments, Cas9 variants with reduced PAM requirements may increase the efficiency of targeting by allowing greater flexibility in selecting gRNA while still enabling insertions to occur near the C-terminus of target genes. In addition, directing Cas9 to cut downstream of the stop codon and relying on error-prone repair to process away the stop codon and place the tag in-frame of the protein of interest may overcome the loss or gain of nucleotides at some junctions.

### Example 5

### Selection Strategies

It was observed that there was a higher chance of successful tagging when the strength of the endogenous promoter is stronger. In addition to tagging with more copies of the maker, as described above, alternative strategies to improve selection and decrease bias in tagging were explored.

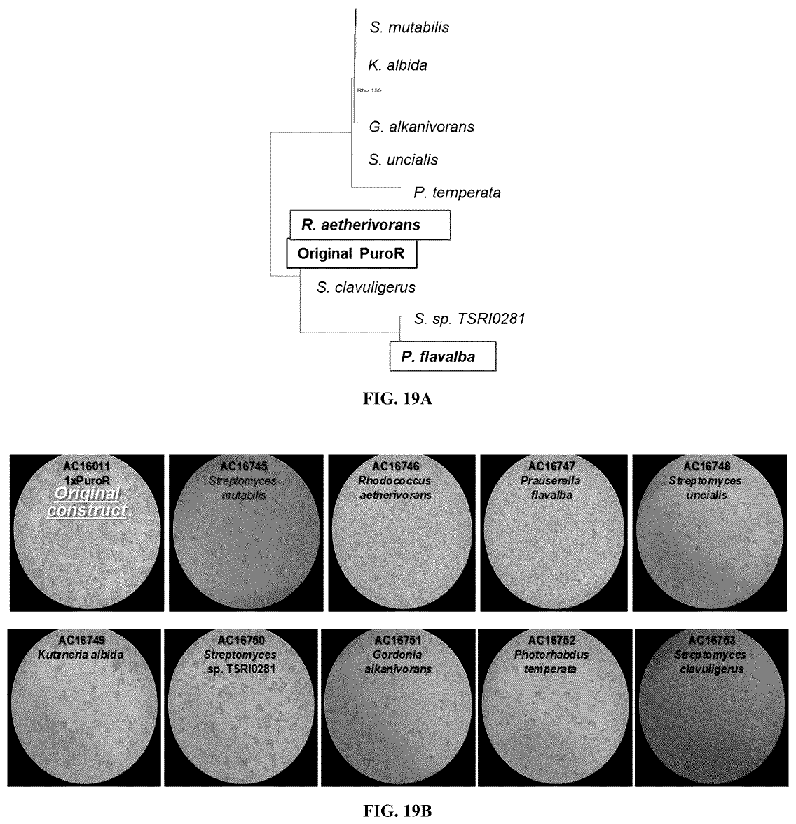

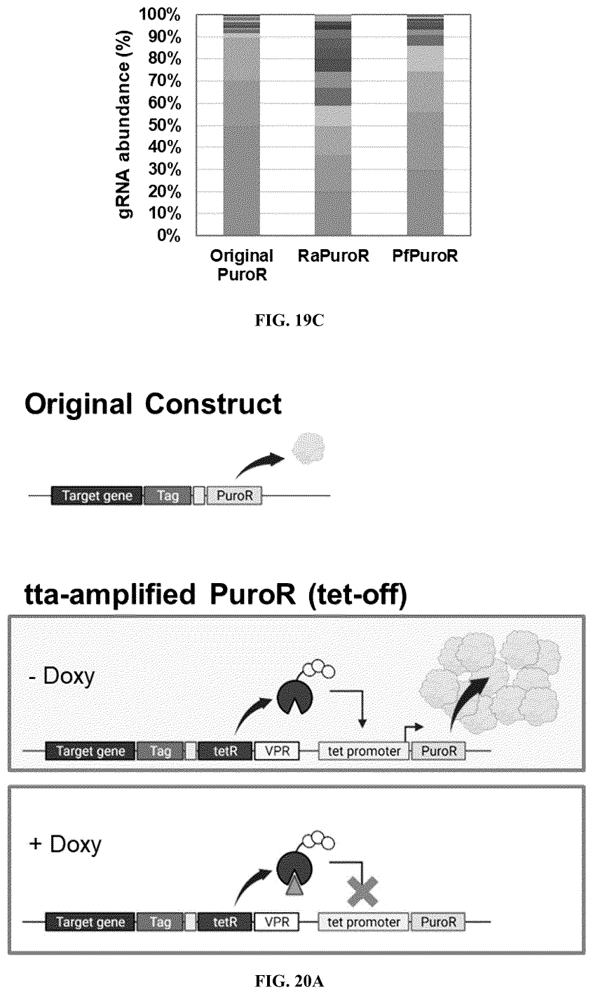

As a way to find more potent puromycin markers metagenomics sequences were searched for homologs of the puromycin resistance gene (FIG. 19A). A variety of puromycin resistance genes with different species of origin were tested for in HITAG for cell growth (FIG. 19B) and proportion of given gRNA in the tagged cells (FIG. 19C). Two top performing puromycin resistance markers Rhodococcus aetherivorans PuroR (RaPuroR) and Prauserella flavalba PuroR (PfPuroR) showed improvements in the distribution of tagged genes as determined by quantifying the relative abundance of gRNAs after applying drug selection in the HITAG approach (FIG. 19C). Only one copy of each puromycin marker was used for these studies. Overall, less bias in tagging is seen when using these puromycin resistance marker homologs.

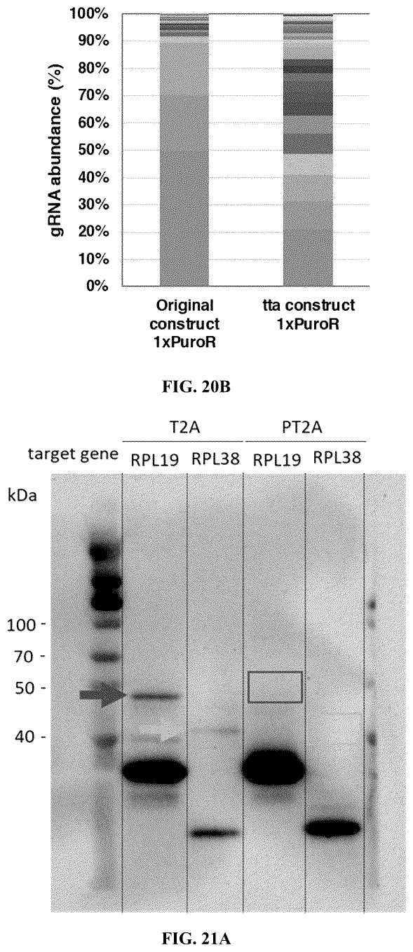

To amplify the puromycin resistance, a synthetic circuit using a transcription factor to control puromycin resistance was designed (FIG. 20A). In the original puromycin resistance construct the puromycin resistance gene is produced from the mRNA of the tagged gene. In the circuit, a transcription factor is produced from the tagged gene which then binds to a promoter driving puromycin resistance, thereby amplifying the signal. A library of targets were simultaneously tagged with either the original puromycin construct or the tta-amplified PuroR circuit configured to drive the expression of a single copy of the puromycin resistance gene. As shown in FIG. 20B, the circuit reduced the bias in tagging as compared to the original construct.

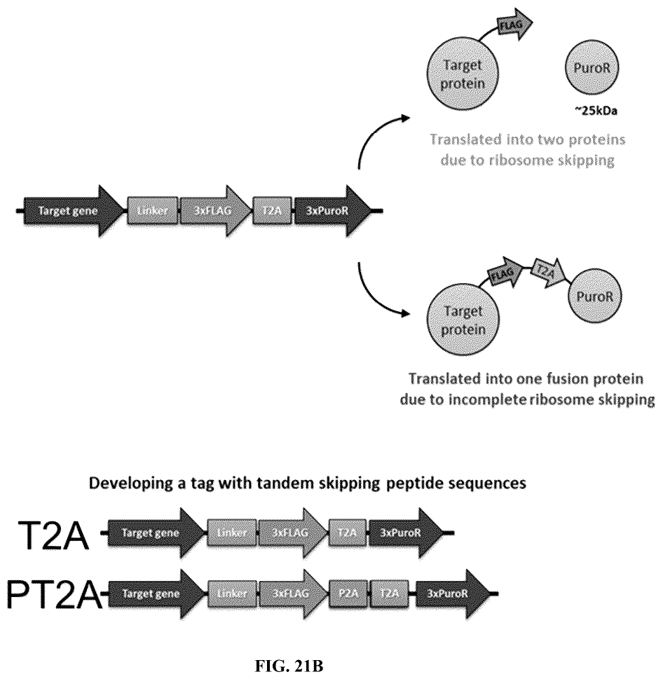

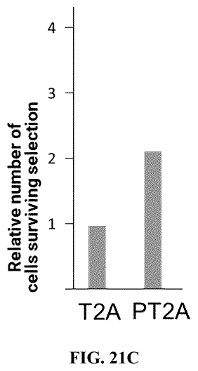

As shown in FIG. 21A, a larger protein product was observed that was likely the puromycin resistance marker fused to our target proteins (FIG. 21B). Inefficient peptide skipping was affecting drug marker stability by leading to unstable fusion proteins and poor drug marker expression. When two copies of the skipping peptide were included in the construct, presence of the fusion protein was abolished and a sharp increase in the amount of tagged protein observed (FIG. 21A), suggesting the use of multiple copies of the skipping peptide eliminated the unwanted PuroR fusions. In addition, it also resulted in improved tagging efficiency since drug marker protein was no longer unstable due to being fused to the tagged protein (FIG. 21C).

### Example 6

### Multiple Allele Tagging

To tag more than a single allele of interest or to tag more than 1 gene at a time in a large population of cells, a new tagging donor plasmid with a different drug marker, which expresses a gene giving resistance to nourseothricin instead of puromycin resistance, was developed for use in a modified tagging pipeline where multiple rounds of tagging occur (FIG. 22A). The first round fuses to one gene with a tag of interest followed by an in-frame puromycin resistance marker. In the second round of tagging, a different tag can be applied if desired or the same as in the previous round can also be used. Furthermore, to enable selection for a second round of tagging, a different drug marker is used (in this case causing resistance to nourseothricin if proper tagging occurs). To enhance nourseothricin resistance from the tagged genes, a similar 3× drug marker approach as in our previous puromycin resistance marker work was employed. Of note, it is possible to also tag different genes of interest in the same cell using this approach. This can be achieved by expressing multiple target gRNAs in one cell. To endow specificity to different target genes during each round, orthogonal Cas9 proteins can be used.

As compared to single allele tagging an increase in the bias in genes being tagged by both rounds is observed (FIG. 22B). Two allele tagging was done in HEK293T cells exposed to two rounds of tagging with the first round containing a pDNR plasmid with a puromycin resistance marker (P) and the second round of tagging containing a pDNR with a nourseothricin (N) resistance marker. To confirm dual allele tagging, PCR was performed looking for PCR junctions between the various drug markers and the C-terminus of the target gene YY1 (FIG. 22C), demonstrating the dual allele tagging occurred. Furthermore, other drug markers can be used and a few demonstrative examples of markers and homologs are listed below.

TABLE 1
Sequences

| SEQ | | |
|---|---|---|
| ID NO | Marker Description | Sequence |
| 1 | PuroR | CTGACTGAATACAAGCCTACTGTCAGGTTGGCTACAAGA |
| | | GACGACGTTCCTAGAGCCGTGAGAACTCTGGCTGCAGCC |
| | | TTCGCCGACTACCCCGCCACGAGACACACCGTTGACCCA |
| | | GATCGGCATATTGAGAGAGTGACTGAACTGCAGGAGCTG |
| | | TTTCTTACAAGAGTTGGCCTCGACATAGGCAAGGTGTGG |
| | | GTGGCGGACGACGGCGCCGCCGTGGCCGTCTGGACCACT |
| | | CCCGAATCAGTTGAGGCTGGCGCCGTATTCGCTGAGATC |
| | | GGCCCGAGAATGGCTGAGCTCAGCGGGAGTAGGCTCGCG |
| | | GCACAGCAGCAAATGGAGGGACTGCTGGCACCACACAG |
| | | GCCCAAAGAACCCGCCTGGTTCCTGGCAACCGTCGGTGT |
| | | ATCTCCCGATCATCAGGGGAAAGGTCTGGGCTCTGCCGT |
| | | AGTGCTCCCTGGCGTGGAGGCAGCTGAGAGAGCAGGAGT |
| | | ACCTGCCTTCTTGGAGACCTCCGCTCCAAGGAATCTTCCC |
| | | TTCTATGAACGGTTGGGCTTCACCGTGACAGCCGACGTG |
| | | GAAGTCCCCGAAGGCCCCCGCACTTGGTGCATGACGAGG |
| | | AAGCCTGGAGCG |
| 2 | RaPuroR Rhodococcus | ACAGAGATCAGACCTGCTGAACCCGCCGATGTGGATCGC |
| | aetherivornas-Nucleic | GCAACAAGAACACTGGCTAGAGCCTTTGCCGACTATCCT |
| | Acid | TTCACCAGACACACCGTGGACGCCCGGGACCACCTGCGG |
| | | AGAGTGGAAGAGCTGCAGCGGCTGTACCTGACCGAGATC |
| | | GGACTGCGGTGCGGCAGAGTGTGGGTCGCCGATGATGCC |
| | | TCCGCCGTGGCCGTGTGGACCACACCTGAGAGCACCGGC |
| | | ATCCCCGAGGCCTTCGAGCGGATCGCCGGCAGAGTGGCC |
| | | GAGCTGAGCGGCGACCGGGCCGACGCCGCTGCTGCCGCC |
| | | GAGGAAGCCCTGGCCCCTCTGAGACCCGTGGGACCTGTG |
| | | TGGTTCCTGGCCACAGTGGCCGTGGACCCCGACAGACAG |
| | | GGCTGGGGACTGGGCGGCGCCGTCCTGGAACCAGGACTG |
| | | AGGGAAGCCAGACAAGCTGGCGTGCCAGCCTACCTGGAA |
| | | ACCAGCAGCGAGAGAAACGTGGCTTTCTACAGAAGACTC |
| | | GGCTTCGACGTTGTGGGATCTGTGACCCTGCCTGGCGAC |
| | | GGCCCTAGAACCTGGGCCATGGTGCGGAACCACACCCAG |
| 3 | PfPuroR Prauserella | TCCGGCGTGATGAAACCTCTGATCCGGGAAGCCACCAGC |
| | flavalba-Nucleic Acid | GCCGACATCGACCCCGCCACCGAGACACTGAGAGATGCC |
| | | TTTGCCGACTACCCCTTCACCAGACACACAATCGCCGCCG |
| | | ATGACCACCTGGGCAGACTGGCCAGAATGCAGAGACTGT |
| | | TCCTGGCTAGAATCGGCCTGCCACATGGCCGGGTGTGGG |
| | | TCAGCGACGACGCCGCCGCCGTGGCCGTGTGGACCACCC |
| | | CTGCTTCTACCGGCCTGGAAAGAGTGTTCACCGAGCTGG |
| | | CCCCTGAGCTGGGCGCCATCGCAGGCGATAGAGCCGCTA |
| | | TTGCCGCTGCCACAGAGGCCGCCCTGGCCCCTCACAGAC |
| | | CTACCACCCCTAGCTGGTTCCTGGGAACCGTGGGAGTGC |
| | | GGCCCGGCCAGCAGGGCCGCGGACTGGGAAGGGCTGTTA |
| | | TCGAGCCTGGCCTGCGGGCCGCTGAGGCCGAAGGCGTGC |
| | | CAGCTTTTCTGGAAACATCTCTGGAAAGCAACGTGGCCC |
| | | TGTACCGGAGATTCGGCTTCGACGTGGTGGCCGAGATCG |
| | | AGCTCCCTCACCACGGCCCTAGAACATGGGCCATGAGCA |
| | | AGAAGCCC |
| 4 | RaPuroR Rhodococcus | TEIRPAEPADVDRATRTLARAFADYPFTRHTVDARDHLRRV |
| | aetherivornas-Amino | EELQRLYLTEIGLRCGRVWVADDASAVAVWTTPESTGIPEA |
| | Acid | FERIAGRVAELSGDRADAAAAAEEALAPLRPVGPVWFLAT |
| | | VAVDPDRQGWGLGGAVLEPGLREARQAGVPAYLETSSERN |
| | | VAFYRRLGFDVVGSVTLPGDGPRTWAMVRNHTQ |
| 5 | PfPuroR Prauserella | SGVMKPLIREATSADIDPATETLRDAFADYPFTRHTIAADDH |
| | flavalba-Amino Acid | LGRLARMQRLFLARIGLPHGRVWVSDDAAAVAVWTTPAST |
| | | GLERVFTELAPELGAIAGDRAAIAAATEAALAPHRPTTPSWF |
| | | LGTVGVRPGQQGRGLGRAVIEPGLRAAEAEGVPAFLETSLE |
| | | SNVALYRRFGFDVVAEIELPHHGPRTWAMSKKP |
| 6 | PuroR Streptomyces | CTGGACCCTCTGCCTCATGTGCGGCCTGCCGCCCAGGAC |
| | mutabilis | GACGTGCCTGCCGCTGTTAGAACACTGGCCAGAGCCTTT |
| | | GCCGATTACCCCTTCACCAGACACGTGGTGGCCGCTGAT |
| | | GGCCACCAGGAGAGAGTGCGGAGATTCCAGGAGCTGTTC |
| | | CTGACAAGGGTGGCCATGGACCACGGCAGAGCCTGGGTG |
| | | ACCGGCGACTGCAGAGCCGTCGCCGCCTGGACCACCCCT |
| | | GAGCGGGACCCCGGCCCAGCCTTCGCCGAAGTGGGACCT |
| | | CTGGTGGGCGACCTGGCTGGCGATCGGGCCGCTGCCCTG |
| | | GCATCTGCCGAGCAGGCCATGGCCCCTCACAGACCTACC |
| | | GACCCAGTGTGGTTCCTGGCCACCGTGGGCGTGGACCCT |
| | | GACGCCCAAGGCGCCGGCCTGGGCACCGCCGTGCTGAGA |
| | | CCCGGCCTGGAAGCCGCTGAACGGGCTAGATTCCCCGCT |
| | | TTTCTCGAGACAAGCGACGAGGGCAACGTGCGCTTCTAC |
| | | ACCCGGCTGGGATTCGAGGTGACAGCCGAAGTCAAGCTG |
| | | CCTGATGACGGCCCCCTGACCTGGTGTATGAGACGGGAA |
| | | CCTGGAAGA |
| 7 | PuroR Streptomyces | ACAACAGATGACAGAGTGCGGCCAGCCACCGAGGCCGA |
| | uncialis | TGTGCCCGCTGCCGTGCGCACCCTGGCCAGAGCCTTTGCC |
| | | GACTACCCCTTCACCCGGCACGTGGTGGCCGCCGACGAC |
| | | CACACAGAAAGAGTGCGGAGATTCCAGGAGCTGTTCCTG |
| | | ACCAGAGTGGGCCTCGCCCACGGCAGAGTCTGGGTGGCC |
| | | GATGATGGCCTGGGAGTGGCAGCCTGGACCACACCTGAG |
| | | CAGGACCCTGCTCCTGGACTGGCCGAGGTGGGACCTCTG |
| | | GTGACCGAACTGGCTGGCGACAGAGCCCCTGCTTTTATG |
| | | GCCGCTGAGGAAGCCCTGGCCCCTCATAGACCCACCGAG |
| | | CCTGTGTGGTTCCTGGCTACAGTGGCCGTGGACCCCGGC |
| | | ATCCAGTCTAAGGGCCTGGGCGCCGCTGTTCTGAGACCA |
| | | GGAATCGAGGCCGCCGACAGAGCCGGCCACCCCGCCTTC |
| | | CTGGAAACCGCCACAGAGCGGAACGTGAGGTTCTACGAG |
| | | AGACTGGGCTTCAGAGTCACCGCCGGCACCACCCTGCCT |
| | | GACGGCGGACCTAGAGTGTGGTGCATGAGACGGGAACCT |
| | | GCCAGC |
| 8 | PuroR Kutzneria albida | GTCGACCTGAGACTGGCTACACTGGAAGATGTGCCCAGA |
| | | GCAGTTGAGACACTGTCTGCCGCCTTCGCCGACTACGCCT |
| | | GGCTGCGCCATACCGTGGCTAGAGATAGACACGCCGAGA |
| | | GAGTGTCCGAGCTGGAACGGCTGTTCGTGGAACACGTGG |
| | | GCCTCAGACACGGCAGAGTGTGGGTCGGCGACGACGGA |
| | | GATGCCGTGGCCGTGTGGACCCACCCTGATACAGACGTG |
| | | GCCGCTGCTTTTGGCGCCATCGCCCCTAGAATGCGGGAA |
| | | CTGGCCGGAGACAGAGCCGAGTACGCCGAGCGGGCCGCT |
| | | GCCGCCCTGGCCCCTCACAGACCAACCGAGCCTGTGTGG |
| | | TTCCTGGGCAGCCTGGGAGTGCGGCCCGAGGCCCAGGGC |
| | | AAGGGCATCGGCGGCGCTATCGTGCAGCCTGGCCTGAGA |
| | | GCCGCCGAAGAGGCCGGCGTGCCAGCCTTTCTGGAAACC |
| | | AGCGAGGAAAGAAACGTGCGGTTCTACAGAAAGCTGGG |
| | | CTTCGAGGTGACCGCCGAGGTGACAATCCCCGACGGCGG |
| | | ACCTACCACCTGGTGCATGAGGCGG |
| 9 | PuroR Streptomyces | CTCCACCACCAGGACACCCCCAGCGTGCGGCCTATCACC |
| | TSRI0281 | GACGCCGATGTGCCCACCGCCGTGGAAACCCTGGCCAGA |
| | | GCCTTCGCCGATTACCCCTATACACGGCATGTGGTGGCCG |
| | | CCGACGACCACGAGGGCAGAATCAGAAGATTCCAGGAG |
| | | CTGTGTCTGACCAGAGTGGGCATGGTCTGCGGCCGGGTG |
| | | TGGGTCGCCGACGCCGGCAGAGCTGTTGCTGTGTGGGCC |
| | | ACACCCGACCAGGACCCTAGCCCTGCTTTTGCCGAAATC |
| | | GGACCTCTGCTGGGCTCTCTGGCCGGAGATAGAGCCGCC |
| | | GCCTTTGAAAGCGCCGAGCAGGCCGTGGCCCCTTACAGA |
| | | CCACAAGAGCCTGCCTGGTTCCTGAACACCGTGGGCGTG |
| | | ACCCCTGAGGCCCAGGGCCAGGGCCTGGGCTCCGCCGTG |
| | | CTGGTGCCAGGCATCGAGGCTGCTGCTAGAGCCGGCTAC |
| | | CCTGTGTTCCTGGAAACAAGCAGCGAGCGGAACGTGAAG |
| | | TTCTACGAGAGACTGGGATTCGAGGTGACAGCCGAAGTG |
| | | GTGCTGCCTGACAATGGCCCTAGAACCTGGTGCATGCGG |
| | | AAGGACCCCAGA |
| 10 | PuroR Gordonia | ACCATCAGACCTCTGATCCGGCCCGCTACACCTGCTGATG |
| | alkanivorans | TGGACGCCGCTGCCGTGACCCTGGGACAGGCCTTCGCCG |
| | | ACTACCCCTTCACAAGACACACCGTGGACAGCCACGACC |
| | | ACGGCGACAGAGTGCGGAGCCTGCAAAGACTGTTCCTGG |
| | | CTGAAATCGGCATGCGGTGCGGCCGGGTGTGGGTGTCTG |
| | | ATGATCTGGCCGCCGTGGCCGTCTGGATCACCCCTAGCTC |
| | | TAGCGGACTGGACGAGGCTTTCGGCGACATCGCCAGCAG |
| | | AGTGGTGGACCTGTACGGCGATAGGGCCGAGATTGCCGC |
| | | CAGAGCCGATGAGGCCACCGCCGGACTGAGACCAGCCG |
| | | AGCCTGTGTGGCATCTGGCCACAGTGGGCGTTGCTCCTCA |
| | | CAGCCAGGGCAGAGGCCTGGGCGCCGCCGTCCTGGAACC |
| | | CGGCCTGGCCGCAGCCCAGCTGGAAGGCCACGTGGCCTA |
| | | TCTGGAAACCAGCAGCCCCGCCAACGTGTCCTTCTACGA |
| | | GCGGCTCGGATTTGAGGTGGCCGGCAAGGTGTCCCTGCC |
| | | TGACGACGGCCCTGAGGTGTGGGCCATGACCTGTGGCAG |
| | | A |
| 11 | PuroR Photorhabdus | AATATGATCGTGCGGGAAAGCAAGGAAATCAGCGAGAT |
| | | CCAGCTGGTGAGATGTGTGCAGACCCTGACAAGAGCTTT |
| | | TGACGGCTACAGCCTGATGCGGCACTTCCTGGCCGAAGA |
| | | TGACCACCAGCAGAGAGTGAGGCGGTACCAGGAGACATT |
| | | CCTGAGAAAGGTGGGAATGACCGTGGGCCACGTGTGGGC |
| | | CGCCGACGATGGCGCTGCTGTGTCCATCTGGACCGCCCCT |
| | | GACATCGAGGACGCCGAGGCAACCTTCGCCCCTCTGTCT |
| | | ATTGAATTCGGAAAAATCGCCGGCACCAGAGAAAAGGTG |
| | | ATGAGAGCCAGCGAGAGCATCATGGCCAAGGAACGCCC |
| | | CAACTTCCCCTGCTGGTTCCTGGGCGCCGTCGCCGTTGAC |
| | | CCCGACTACCAGGGCAAAGGCCTGGGCAGAGCCGTGATC |
| | | GAGCCTGGCCTGGAAAGAGCCGAGTGCGAGGGCTTTCCA |
| | | GTGTTCCTCGAGACATCTGATGATAAGAACGTGCGGATC |
| | | TACGAGAGACTGGGATTCGAGGTGACCGCCGCTTATCAA |
| | | CTGCCTTTCGGCGGACCTATGACCTACGCCATGATCAAGC |
| | | GGGGCATC |
| 12 | PuroR Streptomyces | ACAACCCCTCCTAGCCACCCCGCCGCCGCCAGCAGCGGC |
| | clavuligerus | CCAGTGCGGCCTGCCACAGATGAGGACGTGCCAGCTGCA |
| | | GTTAGAACCCTGGCCAGAGCCTTCGCCGCTTATCCTTACA |
| | | CCCGCCACGTGATCGCTGCCGATGGCCACGAGGAACGGG |
| | | TGCGGAGACTGCAGGAGCTGTTCCTCACCAGAGTGGGCA |
| | | TGGCCTACGGCAGAGTGTGGGTCGGAGGCGAGGGCAGA |
| | | GCCGTGGCCGTGTGGACCACACCTGAGCGGGACCCCTCT |
| | | CCAGGATTCGCCGAAGTGGGACCTCAAATCGCCGAGCTG |
| | | GCCGGCGACAGAGCCGCCGCCTACGAGGCCGTGGAAAG |
| | | AGCTGTGGACCCCTACAGACCCAAGGAACCTGTGTGGTT |
| | | CCTGGGCAGCGTGGCCGTGGACCCTGCCGCCCAGGGACA |
| | | GGGCCTGGGCAGCGCCGTCATCAGACCTGGCCTGGCTGC |
| | | CGCTGATGCCGCTGGTTGTCCTGCTTTTCTGGAAACAGCC |
| | | ACCGAGAGGAACGTGCGGCTGTACGAGAGACTGGGCTTC |
| | | ACCGTGACCGCCGACCTGCCTGGCAGCGACGGCGGCCCC |
| | | AGAATCTGGTGCATGAGACGGGAACCCGGCGCCGGCGG |
| | | A |
| 13 | Puromycin resistance | ACCGCTCTCGATACAGGCGTGCGGCCTGCCGAACCCCAG |
| | marker1 | GACACCCCTCGGGCCGTCAGAACACTGGGCAGAGCCCTG |
| | | GCTGGCTACCCCGCCCTGCGGCACACAGTGGACCCTGAT |
| | | GGACGTGCCGAGAGAGTGACAGCCATCCAGGAGCTGTTC |
| | | TTCACCCGGGTGGGCCTGGAAGCTGGACGGGTGTGGGTC |
| | | GCCGACGGCGGCGACGCCGTGGCCGTGTGGACCACCCCT |
| | | CACAACGGCAATGCCGGCGCCGTGTTCGCCGAGATCGGC |
| | | CCCAGACTGGCCGAGCTGTGTGGCACAAGAGCCGCCGCT |
| | | CAGGAGGCCCTGGACACCGCCCTGGCCCCTCATAGACCA |
| | | ACAGAGCCTGTGTGGTTCCTGGCAACCGTGGGCGTGACC |
| | | CCAGAAAGACAGGGCGCCGGACTGGGCGGAGCCGTGCT |
| | | GAGACCCGGCATCGAGGCCGCCGAGGCTGAAGGCGTTAC |
| | | CGCCTTTCTGGAAACCAGCGACCCCAGAAACCTGCCTTTC |
| | | TACCAAAGACTGGGCTTTGAGATCTCTGCCGACGTGACC |
| | | CCTGCCGATGGTGGACCTAGAACCTGGTGCCTGAGGCGG |
| | | CCTGCTGCTGGCCACGCCTAA |
| 14 | Puromycin resistance | ATTCGGGAAGCCAACCCCGCTGATATCGACCCTGCCACC |
| | marker2 | GAGACACTGTGCGCCGCTTTTGCCGACTACCCCTTCACAA |
| | | GACACACCATCGCAGCCGACGACCACCTGGACCGGCTGG |
| | | CCAGAATGCAGCGGAGATTCCTGAGCAGAATCGGCCTGC |
| | | CTCACGGCCGCGTGTGGGTCTCTGATGACGCCGGCGCCG |
| | | TTGCCGTGTGGACCACCCCTGCCTCCACCGGCATCGGCA |
| | | GGGTGTTCACCGAGCTGGCCCCTGAGCTGGCCGCCATCG |
| | | CTGGCGACAGAGCCGCTATCGCCGCCGCTACAGAGGCCG |
| | | CCCTGGCTCCTCATAGACCCACCACACCTAGCTGGTTCCT |
| | | GGGCACCGTGGGAGTGCGGCCTGATAGACAGGGCAGAG |
| | | GCCTGGGACGGGCCGTGATCGAGCCCGGCCTGAGAGCCG |
| | | CTGAAGCCGAGGGCGTGCCAGCCTTTCTCGAGACAAGCC |
| | | TGGAAGGCAACGTGACCCTGTACCGGAAGCTGGGTTTCG |
| | | AGGTGGTGGCCGAAATCGAGCTGCCACACCACGGACCTA |
| | | GAACCTGGGCCATGAGCAAGGAACCTTAA |
| 15 | Puromycin resistance | ACATCTCCAGCCACAGCTGTGCGGCTGGCAACACGGGCC |
| | marker3 | GATGTGCCCAGAGCCACCGCCACACTGACCAGAGCCTTC |
| | | GCTGATTACCCATTCACCCGGCACACCGTGGCCGCCGAC |
| | | GACCACCTGCGGCGGATCGCCGAGTTCCAGGAGCTGTTT |
| | | GTGGACCGCATCGGCCTGGCCCACGGCAGAGTGTGGGTC |
| | | GGCGACGAGGGAGCCGCTGTTGCTGTGTGGACAACCCCT |
| | | GAGACCGAGGGCGCCGACGCCGTCTTTGCCGAGCTGGCT |
| | | CCTAGATTCGCCGAACTGGCCGGAGATAGAGCCCAGGCC |
| | | TTCGAGCAGGCCGAAGCCGCCCTGGAACCTCACAGACCT |
| | | CAAGGCCCTGCCTGGTTCCTGGGCAGCGTGGGCGTGGAC |
| | | CCCGCTCACCAGGGCAGAGGCCTGGGAAGAGCTGTGCTG |
| | | GCCCCTGGCATCGAGGCCGCTGAAAGAGCCGGCCTGCCC |
| | | GCCTACCTGGAAACCAGCGAGGCCAGAAACGTGGCTTTC |
| | | TACCAGAGACTGGGCTTCGCCGTGTCCGCCGAGGTGGAA |
| | | CTCCCTGGAGGCGGCCCCCTGACCTGGGCCATGACCAGA |
| | | CATGGCTAA |
| 16 | Puromycin resistance | AGCATCAGACCCGCCACCGCCGCCGATATCGACGCCGCT |
| | marker4 | GCCGTGACCCTGCGCGAGGCTTTTACCGACTACCCCTTCA |
| | | GCAGACACACCGTGGCCGCCGACGACCATGCCGCTAGAG |
| | | TGGAACGGGTGCAGCACCTGTTCCTGAGCCGGATCGGCC |
| | | TCCCACACGGCAGAGTGTGGGTGTCCGATGACGTGGCCG |
| | | CCGTCGCAGTGTGGACAACCCCTACCACCACAGACCTGA |
| | | CCGAGGTGTTCGCCGAGCTGGGACCTGAGCTGGCCGAAG |
| | | CCGCTGGCGACAGAGCCGAGGCCGCCGCTGCTGCCGAGG |
| | | CCGCCCTGGCCCCTCTGCGGCCTACAGGCCCTGCCTGGTT |
| | | CCTGGGAGTGGTGGGCGTGCGGCCTGATGCtCAGGGCAG |
| | | GGGCCTGGGCCGGGCCATCATCGAGCCAGGCCTGAGAGC |
| | | CGCCGCTGAAGCCGGCGTTGAGGCCTACCTGGAAACATC |
| | | TCTGGAAACCAACCTGGCCTTTTACAGAAAGCTGGGCTT |
| | | CGAGGTCACAGGCGAGCTGGAACTGCCTGGAGGCGGACC |
| | | TAGAACCTGGGCCATGAGAGCCGCTCCCCCCGTGAAGTA |
| | | A |
| 17 | Puromycin resistance | ACAGGAAACCACAACGGCTCCCCTGCCCCAGGCACAACC |
| | marker5 | ACCACCCCCAAGGCCGACCCCACCGCCGGCACCGCCACC |
| | | GCCCCTGAGGCTGGCCCCGAGGCCAGAGCTGTTGTGCGG |
| | | CCAGCCACAGCCGAGGACGTGCCCAGAGCCGTCAGAACC |
| | | CTGACCCAGGCCTTTGCCAACTACCCCTGGACCAGACAC |
| | | ACCGTGGACGCCGCTGATCACGCCCACCGGATGGAAAGA |
| | | TTTCAGGAGATCTTCCTGACACGGGTGGGCCTGGCTCAC |
| | | GGCAGAGTGTGGGTCGCCGACGACGGCGACGCCGTGGCC |
| | | GTGTGGACCACCCCTGAGACAGTGAACGCCGAAGCCGTG |
| | | TTCGCCGAGCTGGCCCCTGAGTTCGCCGCCCTCGCTGGAG |
| | | ATAGACTGACAGCCTACGAGGAAGCCGAGGCCGCCCTGC |
| | | TGCCCCACCGGCCTACAGAACCTGCATGGTTCCTGGGCA |
| | | CCATCGGTGTGACCCCTGATAGACAGGGCAGCGGACTGG |
| | | GCAGAGCCGTGATTAGACCTGGAATCGCCGCTGCCGAAA |
| | | GAGCCGGCGTGCCTGCTTATCTGGAAACCAGCGACGAGG |
| | | GCAACGTGCGGTTCTACGAGCGCCTGGGCTTCCAAATCA |
| | | CAGCTACCCTGCACCTGCCTGGCAATGGCCCTCGGACAT |
| | | GGAGCATGCTGAGACCACCTAGCCCCACCGCCCCTAGGC |
| | | CTATCACCATGTCTGATCATCCTTAA |
| 18 | Puromycin resistance | CCACAACACCAGGATGCTCCTGATGTGCGGCCTCTGACA |
| | marker6 | GACGCCGATGTGCCCATCGCCGTGGACACCCTGACCAGA |
| | | GCCTTCGTGGGCTACCCCTTCACCAGACATGTGATCGCCG |
| | | CCGACAACCACGAGACAAGGATCAGAAGATTCCAGGAG |
| | | CTGTGTCTGACCCGCATTGGCATGGTGTACGGCAGAGTG |
| | | TGGGTCGCCGACGCCGGCCGGGCCGTGGCCGTCTGGGCC |
| | | ACACCAGACCAGGACCCCAGCCCTGCTTTCGCCGAAATC |
| | | GGCCCTCTGCTCGGCGACCTGACAGGCGATAGAACCGCC |
| | | GCTTATGAGAGCGCCGAGCAGGCCGTGGCCCCTTACAGA |
| | | CCTCAGGAGCCTGCCTGGTTCCTGTCCACCGTGGGAGTG |
| | | ACCCCTGGCGCtCAGGGCAGAGGCCTGGGAACAGCTGTT |
| | | CTGATCCCCGGCATCGAGGAAGCCGAACACGCCGAGTGC |
| | | CCTACCTTTCTGGAAACCTCTAGCGAGAGAAACGTGACC |
| | | TTCTACGAGCGGCTGGGATTTAAGGTGACCGCTGAAGTG |
| | | CTGCTGCCTGGTAGCGGCCCCAGAACATGGTGCATGCGG |
| | | CGGGACCCTAGATAA |
| 19 | Puromycin resistance | ACCCACATCAGACTGGCCACAGCTGACGACATTGCCCCT |
| | marker7 | GCTGCCGACACCCTGGCCGAGGCCTTCGACGGCTATGCC |
| | | TTTACCAGACACACCGTGGCCGCTGATGGCCACCGCGAC |
| | | CGGCTGCGGAGATTCCAGAGACTGTTCCTGGAAAGAATC |
| | | GGCCTCCCCTACGGCAGAGTGTGGGTGGCCGATGACCAC |
| | | GCCGCCGTGGCCGTGTGGACCACACCTGCCACCGCCGCT |
| | | GCTGGAGATGTGTTCGCCGGCGTGGCTGCAGAGCTGATC |
| | | GACATCGCCGGCGACAGAGCCAGACAGCACGCCGACGC |
| | | CGAAGCCGTGATGGCCAGACATAGACCAACAGAGCCTGT |
| | | GTGGTTCCTGGGAACCATCGGAGTGCGGCCTGACAGACA |
| | | GGGCGCCGGCCTGGGCAGGGCCGTTATCGCCCCTGGCCT |
| | | GGCTGAGGCCGCCCGGGAAGGCGTCCCCGCTTTTCTGGA |
| | | AACCTCCATCCGGCGGAACGTGACATGGTACGAGAGCCT |
| | | GGGCTTCAGAGTGACCGCCGATTACGACCTGCCTGATGG |
| | | CGGACCTCACACATGGTCTATGCTGAGACCCCCCAGCGC |
| | | CGAGTAA |
| 20 | Puromycin resistance | ACACCTAGAATCCGGGAAGCCACACCTGCCGACATCGAG |
| | marker8 | CCTGCTGTTGCTACCCTGAGCGCCGCCTTTGCCGATTACC |
| | | CCTTCACCAGACACACCCTGGCTGCTGATGACCACCTGA |
| | | CACGGCTGGCCGACATGCAGAGACTGTTCATCACCCACA |
| | | TTGGACTCCCCCACGGCAGAGTGTGGGTGTCTGATAACG |
| | | CCCACGCCGTGGCCGTCTGGACCACACCAGAAAGCACAG |
| | | CCATCGCCGAAGTGTTCACCGACTTCGCCCCTCAACTGGC |
| | | CCATATCGCCGGCGATAGAGCAGCTATCAGCGCCAGAAC |
| | | CGAGTCTGCTCTGGCCCCTCACAGACCTACCACCCCCACC |
| | | TGGTTTCTGGGAACAGTGGGCGTGCACCCTGAGTCtCAGG |
| | | GCCAGGGCCTGGGAAAAGCCGTGATCGAGCCCGGCCTGC |
| | | GCGCCGCCGACGCCACCGGCACAGAGGCCTTCCTGGAAA |
| | | CCAGCCTGGCCAGCAACGTGACACTGTACCGGAAGCTGG |
| | | GCTTCGACATCGTGGCCGAGATCGACCTGCCTGACGACG |
| | | GCCCTAAGACCTGGGCCATGCGGAGAAAGCCCGCTCCTA |
| | | CCCCAGCCTAA |
| 21 | Puromycin resistance | CCAGCCACAACACCTAGCGTGCGCCCCACCCGGCACGAC |
| | marker9 | GATGTGCCTGCTGGCGTGCGGGTGCTGGCTAGAGCCTTC |
| | | GCCGACTACCCCTTCACCAGACACGTGGTCGCCGCTGAT |
| | | GATCACCCCAGAAGAGTGAGGCGGCTGCAGGAGCTGTTC |
| | | CTGGCCAGAATCGCCCTGCCTTACGGCAGATCCTGGGTC |
| | | ACCGACGACGGCCTGGCCGTGGCCGCCTGGACCACCCCT |
| | | GAGCGGGACCCAGAACCTGCCTTTGCCGAAATCGCCCCT |
| | | GTGATCGCCGAACTGGCCGGATCTAGATGGGCCGCTTAT |
| | | CAGGCCGCCGAGGAAGCCCTGGCACCACATAGACCTGCC |
| | | CACCCTGTGTGGTTCCTGGCTACAGTGGGCGTTGACCCTG |
| | | ACGCtCAGGGCCAAGGAAGAGGCGCCGCTGTGCTGAGAC |
| | | CCGGCCTGGAAGCCGCCGAGGCCGCCGGCCTGCCTGCTT |
| | | TTCTCGAGACAAGCGACCCCGGCAACGTGCGGTTCTACG |
| | | AGAGACTGGGCTTCACCGTGACCGCCGAGGTGCCCCTGC |
| | | CTGATGGCGGACCTCTGACCTGGTGCATGCTGAGAGCCC |
| | | CTGGCAGATAA |
| 22 | Puromycin resistance | TCCGTGACAATCCCTCCAACCAGAAGAACAACCCATGAT |
| | marker10 | GACGTGCCCGCCTGCGTGGAAGTTCTGACCCGGGCCTTT |
| | | GCCGACTACCCCTTCACCAGACACGTGGTGGCCGCTGAT |
| | | GACCACGAGAGAAGGGTGCGGAGACTGCAGGAGCTGTT |
| | | CCTGACCAGAGTGGCCCTGAGACACGGACGGAGCTGGGT |
| | | CACCGACGATAGACTGGCTGTGGCAGCCTGGACCACCCC |
| | | TGAACAGGACCCCAGCCCTGCCTTCGCCGAAATCGGCAG |
| | | CCTGCTGCCTGAGCTGGCCGGCGACAGAGCCGCTGCCTA |
| | | CGAGGCCGCCGAGGAAGCCCTGGCCCCTCACAGACCTAC |
| | | CCACCCTGTGTGGTTCCTGGCCACCGTGGGCGTGGCCCCT |
| | | GAGGCtCAGGGCAGAGGCCGGGGCGCCGCCGTGCTGCGG |
| | | CCTGGCCTGGAAGCCGCTGAAGCTACAGGCTTCCCCGCT |
| | | TTCCTCGAGACATCTGATGCCAGAAACGTGCGGTTCTAC |
| | | GAGCGGCTGGGCTTTACCGTCACCGCCGAGGTGCCTCTG |
| | | CCAGACGGCGGACCTCTGACATGGGGAATGACAAGAAG |
| | | CCCCGGCCGCTAA |
| 23 | Puromycin resistance | ACAACCAACGCCCCTGTGGTCAGACCTGCTACACGGGAC |
| | marker11 | GACCTGCCAAGAGCCCTGCGGACCCTGCAGAGAGCCTTT |
| | | GCCGATTACGCCTTCACCCGCCACACCATCGCCGCTGATG |
| | | GCCATCTGGACCGGCTGCACAGATTCAACGAGCTGTTCG |
| | | TGACAAGAATCGGCCTGGAACACGGCAGAGTGTGGGTGG |
| | | CCGACGGCGGCGCCGCTGTTGCTGTGTGGACCACACCTG |
| | | AGACAGCCGAGGCCGGAAGCGTGTTCGCCGAACTGGGAC |
| | | CTCTGTTTGCTGAGATCGCCGGCGACAGAGCCGAAATCT |
| | | TCGCCCAGACCGAGGCCGCtCTGGGACCTCACCGGCCCAC |
| | | CGGCCCTGTGTGGTTCCTGGGATCTGTGGGAGTGGACCCT |
| | | GATAGACAGGGCAGGGGCCTCGGCGGAGCCGTGATCAG |
| | | ACCCGGCCTGGAAGCTGCCGATGCCGCCGGCGTGCCCGC |
| | | CTTCCTGGAAACCAGCGACGAGAGAAATGTGCGGTTCTA |
| | | CGAGCGGCTGGGCTTCGAGGTGACCGCCGAGTGCGTGCT |
| | | GCCTGGCGGCGGACCTAGAACCTGGTCCATGAGCAGAAA |
| | | GCCTGTCAGCTAA |
| 24 | Puromycin resistance | ACAACCAGCACCCCTGCCGTGCGGCCCGCTACACGCGAC |
| | marker12 | GACCTGCCTAGAGCCCTGCGGACCCTGAGAAGGGCCTTC |
| | | AGCGACTACCCCTTCACTCGGCACACCATCGCCGCTGAT |
| | | GGCCACCTGGACAGACTGCACAGATTCAACGAGCTGTTC |
| | | CTGACCAGAATCGGCCTGGAACACGGCAGAGTGTGGGTC |
| | | GCCGATGGAGGCGCCGCTGTGGCCGCCTGGACCACACCT |
| | | GAAACCGCCGAGGCCGGATCTGTTTTCGCCGAGCTGGGA |
| | | CCTCTGTTTGCCGAGATCGCCGGCGACCGGGCCGAAATC |
| | | TTCGCCCAGACCGAGGCCGCtCTGGGACCTCACCGGCCTA |
| | | CAGGCCCTGTGTGGTTCCTGGGAAGCGTGGGCGTGGACC |
| | | CTGATAGACAGGGCAGAGGCCTCGGCGGAGCCGTGATCA |
| | | GACCAGGCCTGGAAGCTGCCGACGCCGCTGGCGTGCCTG |
| | | CTTTTCTGGAAACATCTGATGAGAGAAACGTGCGGTTCT |
| | | ACGAGCATCTGGGCTTCGAGGTGACCGCCGAGTGCGTGC |
| | | TGCCCGGCGGCGGACCAAGAACCTGGAGCATGAGCAGA |
| | | AAGCCTGTGTCCTAA |
| 25 | Puromycin resistance | ACCATGAGCACACCTGCCGTGCGGCCTGCTACACACGAC |
| | marker13 | GACCTGCCTAGAGCTCTGAGAACCCTGCAGAGAGCCTTC |
| | | AGCGACTACCCCTTCACCAGACACACCATCGCCGCTGAT |
| | | GACCACCTGGACCGGCTGCACAGGTTCAACGAGCTGTTC |
| | | GTGACAAGAATCGGCCTGGAACACGGCAGAGTGTGGGTG |
| | | GCCGATGGCGGAGCCGCCGTCGCCGTCTGGACCACACCT |
| | | GAGACAGCCGAAGCCGGCAGCGTGTTCGCCGAGCTGGGA |
| | | CCTCTGTTTGCTGAGATCGCCGGAGATAGAGCTGAAATC |
| | | AGCGCCCAGACCGAGGCCGCtCTGGGACCACATCGCCCC |
| | | ACCGGCCCAGTGTGGTTCCTGGGAAGCGTGGGCGTGGAC |
| | | CCCGACAGACAGGGCAGAGGCCTGGGCGGCGCCGTGATC |
| | | AGACCTGGCCTCGAAGCCGCCGACGCCGCCGGCGTTCCC |
| | | GCTTTTCTGGAAACCTCTGATGAGCGGAACGTGCGGTTCT |
| | | ACGAGCACCTGGGCTTCGAGGTGACCGCCGAGTGCGTGC |
| | | TGCCTGGCGGCGGCCCTCGGACCTGGTCCATGTCTAGAA |
| | | AGCCTGGACCTTAA |
| 26 | Puromycin resistance | ACCACCAATACCCCTGTGGTGCGGCCTGCCACCAGAGAT |
| | marker14 | GATCTGCCAAGAGCCCTGAGAACCCTGCAAAGAGCCTTC |
| | | GCCGACTACGCCTTCACACGCCACACCATCGCCGCTGAC |
| | | GGCCACCTGGACCGGCTGCACAGATTCAACGAGCTGTTC |
| | | GTGACCAGAATCGGCCTGGAACATGGAAGAGTGTGGGTC |
| | | GCCGACGACGGCGACGCCGTGGCCGTTTGGACCACACCT |
| | | GAGACAGCCGCTGCCGGCAACGTGTTCGCCGAGGTGGGA |
| | | CCTCTGTTTGCCGAGATCGCCGGAGATAGGGCTGAAATC |
| | | AGCGCCCAGGCCGAAGCTACCATGGGACCTCACCGGCCT |
| | | ACAGAGCCTGTGTGGTTCCTGGGCTCCGTGGGCGTGGAC |
| | | CCCGACAGACAGGGCAGAGGCCTGGGAGGCGCCGTGAT |
| | | CAGACCTGGACTCGAAGCCGCCGACGCCGCTGGCGTCCC |
| | | CGCCTTTCTGGAAACATCTGACGAGCGGAACGTGCGGTT |
| | | CTACGAGAGACTGGGCTTCCAGGTGACCGCCGATTACGT |
| | | GCTGCCCGGCGGCGGACCTAGAACATGGGCCATGAGCAG |
| | | AAAGCCTGGCGCTTAA |
| 27 | Puromycin resistance | AGCCAACATCAGAACGCCCCTAGCGTGCGGCCAATCACC |
| | marker 15 | GACGCCGACGTGCCCGCTGCAGTGGACACCCTGGCCAGA |
| | | GCCTTCGCCGACTACCCTTACACCAGACACGTGATCGCC |
| | | GCCGACGGCCACGAGGAACGGATTAGAAGATACCAGCA |
| | | GCTGTGCCTGACCCGGATCGGCATGGTGTACGGCAGAGT |
| | | GTGGGTCGCCGATGAGGGCAGAGCCGTCGCCGTGTGGGC |
| | | CGTTCCTGGTCAGGACCCTAGCCCTGCTTTCGCTGAACTG |
| | | GGACCTATCCTGGGCGAGCTGTCTGGCGACAGAGCCGCC |
| | | GTGTCCGCCACAGCCGATGCCGCTATGGCCCCTTATAGA |
| | | CCCAAGGAACCTGGCTGGTTCCTGGAAACAGTGGCTACA |
| | | GCCCCAGAGGCtCAGGGCAAAGGCCTGGGATCTGCCGTG |
| | | CTGATCCCCGGCATCCAGGAGGCCGAGAGAGCCGGATGT |
| | | CCTGCCTTCCTGGAAACCAGCAGCGAGGCTAATGTGCGG |
| | | TTCTACGAGAGGCTCGGATTTAAGGTGACCGCCGATGTG |
| | | CAGCTGCCTGGCAACGGCCCCAGAACCTGGTGCATGCGC |
| | | CGGGACCCCCACTAA |
| 28 | Puromycin resistance | CCCACCTCCTGCAGCCCTAGCGTGCGGCCTGCCACACGG |
| | marker16 | GCCGACCTGCCTAGAATCCTGAGAACCCTGGAAGGCGCT |
| | | TTTACCGACTACCCACTGACAAGACACACCCTGGCCGCA |
| | | GATGGCCACGCCGACAGACTGCGGAGATTCAACGAGCTG |
| | | TTCGTGACCCGGGTGGGCCTGGACCACGGCAGAGTGTGG |
| | | GTGGCCGATGGCGGCGCCGCCGTTGCCGTCTGGACAACC |
| | | CCAGAAACCGCCGAGGCCGGCGACGTGTTCGGCGAGCTG |
| | | GGACCTCGGTTCGCCGAGATCGCCGGAGATCGCGCCGAA |
| | | ATCAGCGCCCAGACCGAGGCCGCTATGGGCGTGCACAGA |
| | | CCTACAGAGCCTGTGTGGTTCCTGGGCACCGTGGGTGTG |
| | | GACCCCGGAAGACAGGGCCAGGGCCTGGGCGCCGCCGT |
| | | GATCAGACCCGGACTCGAAGCCACAGGCGCTGCTGGCGT |
| | | CCCTGCTTTTCTGGAAACCTCTGACGCCAGAAACGTGAG |
| | | GTTCTACGAGCGGCTGGGCTTCGAGGTGACCGCTGATTA |
| | | CCCCCTGCCCGGCGGCGGACCTAGAACATGGGCCATGAC |
| | | CCATAAGCCTGGCGCCTAA |
| 29 | Puromycin resistance | ACCGAGCAGGCCCCTGCTGTGCGGGCAGCCACACGGGAA |
| | marker17 | GATCTGCCAAGAGCCGTGCGGACACTGGGCAGAGCTTTT |
| | | CTGCACTACCCCCTGACCAGGCATACAATCGCCGCCGAT |
| | | GACCACGCCGCCAGACTGGAAAGATTCAACCACCTGTTT |
| | | GTCAGCAGAATCGGACTCGAGCACGGCAGAGTGTGGGTG |
| | | TCTGATGATTGCGCCGCCGTGGCCGCTTGGACCACCCCTG |
| | | CCACCGACGCCGCCGCCGTTTTCGGCGAGATCGGCCCTG |
| | | AGCTGGAAAGACTGGCCGGCGACAGAGCCCCATTCGCCG |
| | | CTCGGGCCGAGGAAACCATGCGGCCCCACAGACCTACTG |
| | | TGCCTACATGGTTCCTGGCTACAATCGGCGTGGACCCTGG |
| | | CAGACAGGGACAAGGCCTGGGAAGAGCCGTCGTGCTGCC |
| | | TGGAGTGGAAGCCGCTGAGCGCGCTGGCGTGCCCGCCTT |
| | | CCTGGAAACCAGCGACGAGCGGAACGTGCGGTTCTACCA |
| | | GGGCCTGGGCTTCGAGGTGACCGCCGACTACGCCCTGCC |
| | | TGACGGCGGCCCCAGAACCTGGGCCATGACCAGAGAGCC |
| | | TGGCGCCTAA |
| 30 | Puromycin resistance | AGCGTGACAACCCCTCCAGCCAGACCAACAACACATGAT |
| | marker18 | GATGTGCCTGCATGCGTGGAAGTGCTGACCAGAGCCTTC |
| | | GCCGATTACCCCTTCACCCGGCACGTGGTCGCCGCCGAC |
| | | GACCACAAGTGGCGGGTGCGGAGACTGCAGGAGCTGTTC |
| | | CTCGCCAGAGTGGCCCTGAGATACGGCAGGTCTTGGGTC |
| | | ACCGACGACAGACTGGCCGTTGCCGCCTGGACCACCCCT |
| | | GAGCAGGACCTGTCCCCTGCCTTTGCCGAGATCGGCAGC |
| | | CTGCTGCCTGAACTGGCCGGAGATAGAGCCGCTGCTTAT |
| | | GAGGCCGCCGAGGAAGCTCTGGCCCCTCACAGACCTACA |
| | | CACCCCGTGTGGTTCCTGGCTACAGTGGGCGTGGCTCCTG |
| | | AGGCtCAGGGCCGGGGCCGCGGCGCCGCTGTGCTGCGGC |
| | | CTGGACTGGAAGCCGCTGAGGCCGCCGGCTTCCCCGCCT |
| | | TCCTGGAAACCAGCGACGCCAGAAACGTGCGGTTCTACG |
| | | AGAGACTGGGATTTACCGTGACCGCCGAGGTGCCCCTGC |
| | | CCGACGGCGGCCCTCTGACTTGGGGCATGACCAGAAGCC |
| | | CTGGCAGATAA |
| 31 | Puromycin resistance | ATTATCAGACCCGCTACAGCCGCCGACGTGGACGCCGCC |
| | marker 19 | GTGACCACCCTGTCTATGGCCTTCGCCGATTACCCCTTCA |
| | | CCCGGCACACAATCGCCGCCGACGACCACGCCGGCAGAC |
| | | TGGCTAGAAGCCAGAGACTGTTTCTGACCAGAATCGGCC |
| | | TGCCCCACGGCAGGGTGTGGGTGTCCGACCATGCCGAGG |
| | | CCGTCGCCGCTTGGACAACCCCTGATGCCGCAGACCTGG |
| | | GCAGAGTGTTCGCCGATGTGGCCCCAGAGCTGGCCGAGC |
| | | TGGCCGGAGATAGAGCCGAAATCGCCGCTGAGAGCGAG |
| | | GCCGCTCTGGCTCCTTTTAGACCAACAGGCCCTGCCTGGT |
| | | TCCTGGGTACAGTGGGAGTGCGGCCTGGAAACCAGGGCC |
| | | GGGGCCTGGGCCGCGCCGTCATCCAACCTGGCCTCGACG |
| | | CCGCTGAAGCCGACGGCGTGCAGGCCTACCTGGAAACCA |
| | | GCACCGAGCGGAACGTGGAACTGTACCGGAAGCTGGGCT |
| | | TCGAGGTTGTGGGCGAGGTGGAACTGCCTAGAGGCGGAC |
| | | CTAAGACCTGGGCCATGCGGAGAGGCTGCTAA |
| 32 | Puromycin resistance | CGGACCGAGCAGTCTAGCCAACCTGCTCCACCTACCGTG |
| | marker20 | AGGTCCGCCACACCCGCTGATATCCCCAGAGCCACCAGA |
| | | ACaCTGGGCAGAGCCTTTGCCGACTACGCCTGGACCCGGC |
| | | ACACCATCGACGCCAGAGACCACGAACAGAGAGTGCGG |
| | | GGAATGCAGGAGCTGTTCCTGACCCACATCGGCCTCCCC |
| | | CACGGCCGCGTGTGGATCGCCGACGAGGGCGCCGCTGTG |
| | | GCCGTGTGGACAACACCTGCCACCGATGCCGGCCCTGCC |
| | | TTCGCTGAACTGGCCCCTAGATTCGCCGATCTGGCCGGCG |
| | | ACAGAGCCGCCGCCTACGCCGCTGCCGACGCCGCCCTGG |
| | | CCCCACATAGACCCGTCGAGCCTGTGTGGTTCCTGGGTAC |
| | | AGTGGGCGTGGACCCCGACAGCCAGGGCAGAGGCCTGG |
| | | GCGGCGCCGTGATCCGGCCTGGACTGGCTGCCGCCGATA |
| | | GAGCAGGCGTTCCTGCTTTTCTGGAAACCAGCGAGAAGC |
| | | GGAACGTGGGATTCTACGAGCGGCTGGGCTTCAGAGTGA |
| | | CCGCCACAGTGGACCTGCCTGACGGCGGACCTACAACCT |
| | | GGGCCATGCTGAGAGATCCTGGCGCTTAA |
| 33 | NATMX (noursethricin | ACCACTCTTGACGACACGGCTTACCGGTACCGCACCAGT |
| | resistance marker) | GTCCCGGGGGACGCCGAGGCCATCGAGGCACTGGATGGG |
| | | TCCTTCACCACCGACACCGTCTTCCGCGTCACCGCCACCG |
| | | GGGACGGCTTCACCCTGCGGGAGGTGCCGGTGGACCCGC |
| | | CCCTGACCAAGGTGTTCCCCGACGACGAATCGGACGACG |
| | | AATCGGACGACGGGGAGGACGGCGACCCGGACTCCCGG |
| | | ACGTTCGTCGCGTACGGGGACGACGGCGACCTGGCGGGC |
| | | TTCGTGGTTGTCTCGTACTCCGGCTGGAACCGCCGGCTGA |
| | | CCGTCGAGGACATCGAGGTCGCCCCGGAGCACCGGGGGC |
| | | ACGGGGTCGGGCGCGCGTTGATGGGGCTCGCGACGGAGT |
| | | TCGCCCGCGAGCGGGGCGCCGGGCACCTCTGGCTGGAGG |
| | | TCACCAACGTCAACGCACCGGCGATCCACGCGTACCGGC |
| | | GGATGGGGTTCACCCTCTGCGGCCTGGACACCGCCCTGT |
| | | ACGACGGCACCGCCTCGGACGGCGAGCAGGCGCTCTACA |
| | | TGAGCATGCCCTGCCCC |
| 34 | NATMX resistant | ACAACCGTGGACGACATGGCCTACGAGTTCAGAACCGCC |
| | marker1 | AGACCTGAGGATACCGAGGCCATTGAAGCCCTGGATGGC |
| | | AGCTTCACCACCCACACCATCTTCCAAGTGGCCGTGACA |
| | | GAAACCGGCTTCGCCCTCCAGGAGATCCCTGTGGACCCC |
| | | CCCATCCATAAGGTGTTCCCCGCTGAAAACACAGCTGAT |
| | | GCCCCTGTTGCCGAGGGAGATCCTTCTAGCAGAACCTTTG |
| | | TGGCCGTGGGAACCGACGGCAGCCTGGCTGGATTCGCTA |
| | | CAGTGTCCTACGCCAGCTGGAATCGGAGACTGGCCATCG |
| | | AGGACATCGAAGTGGTCCCCGCCCACAGAGGCCGCGGCG |
| | | TGGGCAGAGCCCTGATCGGCCACGCCGTGACCTTCGCCA |
| | | GAGAGAGCGGCGCCGGCCACATCTGGCTGGAAGTGACA |
| | | AACATCAACACCCCTGCTATCCACGCCTATCAGCGGATG |
| | | GGCTTTACCTTCTGCGGCCTGGACACAACACTGTACGAC |
| | | GGCACCCCATCTAGCGGCGAGCAGGCCCTGTATATGAGC |
| | | ATGCCTTGTCCTTAA |
| 35 | NATMX resistant | ACCACCGCCGATGAGACAACCTACGAGTTCCGGGCCGCT |
| | marker2 | AGACGGGAAGATTTCGAGGCCATCGACGCCCTGGACGGC |
| | | AGCTTCACCACCAGCACCGTGTTCAGAGTGGACGTGACA |
| | | GGCGACGGATTTGCCCTGAGAGAGGTCCCCGTGGACCCT |
| | | CCACTGACCAAGGTGTTCCCCGAGGACGAGTCTGAAGGC |
| | | GCCGACGGCGCCGACAGCGGCTCTAGAACCTTCGTGGCT |
| | | GTTGGAGCTGACGGCGAGCTGGCCGGATTCGCCGCCGTG |
| | | TCCTACAGCCCTTGGAACCGGCGGCTGACAGTGGAAGAT |
| | | ATCGAGGTGGCCCCTGGCCACAGAAATAGAGGCGTGGGC |
| | | CACGCCCTCATGGGCCACGCCGTGGACTTCGCCAGAGAA |
| | | TGTGGCGCTGGACATGTGTGGCTGGAAGTGACCAACGTC |
| | | AACGCCCCTGCTATCCACGCATATAGAAGGATGGGCTTT |
| | | GCCTTCTGCGGCCTGGATACAGCCCTGTACCAGGGCACA |
| | | GAGAGCGAGGGCGAACAGGCCATCTACATGAGCATGCCT |
| | | TGCCCCTAA |
| 36 | NATMX resistant | ACCACCATCGGAGCCATGGACTACGAGTTCAGAACAGCC |
| | marker3 | AGACCTCCCGATACCCCTGCTATGGAAGCCCTGGATGGA |
| | | TCTTTTACCACAAGAACCATCTTCCACGTGGCTGCTACAG |
| | | AGGATGGCTTCGCCCTCCAGGAGATCCCCGTGGACCCTC |
| | | CACTGCACAAGGCCTTCCCCGCTGGCGACAGCGACGCCG |
| | | ATGCCGACGACGGACTGACCACAGAAGAGGACCCCAAT |
| | | AGCAGAACCTTCGTGGCCGTGGGACCTGATGGTTGTCTG |
| | | GCCGGATTTGCCGCTGTCTCCTACGCCCCTTGGAACCGGC |
| | | GGCTGGCCATTGAGGACATCGAGGTGGCCCCTGCACATA |
| | | GAAGCCAGGGCCTGGGCAGAGCCCTGATGGCCCACGCCG |
| | | CCGACTTCGCCAGGGAAAGAGGCGCCGGCCACATCTGGC |
| | | TGGAAGTGACCAACATCAACGCCCCAGCTATCCACGCCT |
| | | ACCGGAGAATGGGCTTCACATTCTGCGGCCTGGACACCA |
| | | CACTGTACGACGGCACCCCTAGCAGCGGCGAGAGAGCCC |
| | | TGTATATGTCTATGCCTTGCCCT TAA |
| 37 | NATMX resistant | ACAACCGTTGGCGACACCGCTTACCGCTACCGGATCGCC |
| | marker4 | GCTGCTGGAGATATCGAGGCCATCAGAGCCCTGGATGAT |
| | | AGCTTCACCACACACACCGTGTTCAGAGTGACCGTGACA |
| | | GAGGAAGCCTTCGCCCTGCGGGAAATCCCCGTGGAACCC |
| | | CCCCTGACCAAGGTGTTCCCTAAGAACGAGCCTGACGAC |
| | | GAGGACGACGCCGACAGCAGAGCCTTTGTGGCCCACGGC |
| | | GCCGCTGGCGACCTGGCCGGATTTGCCGCCGTGTCCTAC |
| | | AGCGGCTGGAATAGAAGGCTGACAATCGAGAACATTGTG |
| | | GTCGCCCCTCCACATAGAGGAAGAGGCGTGGGCAGAGCC |
| | | CTGATCGAGCTGGCCAAGAAATTCGCTAGAGAGAGAGAT |
| | | GCCGGCCACCTGTGGCTGGAAGTGACCAACATCAACGCC |
| | | CCTGCAATCCACGCCTATCGGAGAATGGGCTTCGCCTTCT |
| | | GCGGCCTGGACACCACCCTGTACGAGGGCACACCTAGCA |
| | | AGGGCGAACAGGCCCTCTACATGTCTATGCCTTGTCTGTA |
| | | A |
| 38 | NATMX resistant | ACAACAGCCGGAGATACACCTTACCGCTACAGAGTGGCC |
| | marker5 | GCTCCTGAGGACACCGAGGCCGTGAGAGCCCTGGACGCC |
| | | TCCTTCACCACCGACACCGTCTTTCAGGTGACCGTTACAG |
| | | AGGAAGGCTTCGCCCTGCGGGAAATCAGAATGGAACTGC |
| | | CTCTGACAAAGGTGTTCCCCGAGGACGAGCCCGACGACG |
| | | ACGCCGAGGACGATGCTGATAGCCGGACCTTCATCGCCC |
| | | ATGATGCCGCCGGCGACCTGGCTGGCTTCGTGACAGTGG |
| | | CTTATTCTGGCTGGAATAGACGGCTGACCGTGGAAGATA |
| | | TCGCCGTGGTGCCCCAGCACAGAGGCAGGGGAGTGGGA |
| | | AGAGCCCTGGTGGGCCTGGCCAGAAAGTTCGCTAGAGAG |
| | | AGAGGCGCCGGCCACCTGTGGCTGGAAGTGACCAACATC |
| | | AACGCCCCTGCCATCCACGCCTACCGGAGAATGGGCTTT |
| | | GCCTTCTGCGGCCTGGACACCACCCTGTACGAGGGCACC |
| | | CCTAGCAGAGGCGAGCAGGCCCTGTATATGAGCATGCCA |
| | | TGTCACTAA |
| 39 | NATMX resistant | ACAACCGTGGACACCATGAACTACGAGTTCAGAACCGCC |
| | marker6 | CGACCTGAGGATACCGAGGCCATCGACGCCCTGGATGGC |
| | | AGCTTCACCACCAGAACAATCTTCCACGTGGCCGTGACA |
| | | GAAGGCGGATTCGTCCTGCAGGAGATCCCCGTTGATCCT |
| | | CCAATCCATAAGGTGTTCCCTGCTGAAGATACCGACGAC |
| | | GGCAACAGCCCAGCCGCTGGCGAGGACCCCAATTCTAGA |
| | | ACCTTCGTGGCTATCGGCGCCGACGGCGGCCTGGCCGGC |
| | | TTTGCCGCTGTGTCTTACGCCCCTTGGAACGGCAGACTGA |
| | | CAATCGAGGACATCGAGGTGGCCCCCGCTCACCGGGGAC |
| | | AGGGCGTGGGCAGAGCCCTCGTGGGCCACGCCGCCGAGT |
| | | TCGCCCGGGAAAGAGGAGCCAGACACATCTGGCTGGAA |
| | | GTGACCAACATCAACGCCCCTGCCATTCACGCCTACAGA |
| | | AGAATGGGCTTCAGCTTTTGTGGCCTGGACATGGCCCTGT |
| | | ATGACGGAACACCTAGCAGCGGCGAACAGGCACTGTATA |
| | | TGAGCCGGTCCTGCCTGTAA |
| 40 | NATMX resistant | ACCACCGCCGACGATACACCTTACGAAATCAGAATCGCC |
| | marker7 | GCCAGAGAAGATGCCGGAGCCCTGAAGGCCCTGGACGG |
| | | CTCCTTCACAACAACCACCGTGTTCCACGTGGAAACCAG |
| | | CGAGAACGGCTTCGCCCTGAGAGAGTCTCTGATTGAGCC |
| | | TCCACTGACAAAGGTGTTCCCCGAGGATGATCAGGGCGA |
| | | CAGCGACGGCGACGACGAGAGAGGCAGAGTGGACCAGA |
| | | ATAGCAGAACCTTCCTGGCtCTGGGCGCTGATGGCAGCCT |
| | | GGCTGGATTTGTGTCCGTGGCCTATGCCCCTTGGAACCGG |
| | | AGACTGACCATCGAGGACATCGAGGTGGCTCCTGAACAC |
| | | CGGGGCCGGGGCGTGGGAAGAGCCCTGGTTGGACGGGCT |
| | | GAAGGCTTCGCTAGAGAGAGAGGCGCCGGCCACATCTGG |
| | | CTGGAAGTGACCAACGTCAACGTGCCAGCCGTGCGGGCC |
| | | TACAGAAGGATGGGCTTTGTGCTGTGCGGCCTGGACACA |
| | | TCTCTGTACGAGTTCACCGCCAGCGCCGGCGAGTACGCC |
| | | CTGTATATGCGAAAACCTTGTAGACCCCACAGACCTGCC |
| | | CTCACCCCCAGCCCCACCGAGACACCTCTGACCGCCGCT |
| | | CATAGATCTGCCGAAAGCAGCACAAGCTAA |
| 41 | NATMX resistant | ACAACAGTTGACGATACAACCTACGCCCTGAGAACCGCC |
| | marker8 | CGGCCTGAGGACGCCGAAGCTATTGAGGCCCTGGACGGC |
| | | TCTTTTACAACAAGCACCGTCTTTAGAGTGGAAATCGCCG |
| | | AGAATGGCTTCACCCTGCGGGAAACCCCTGTGGACCCCC |
| | | CCCTGACAAAGGTGTTCCCAGAAGATGAGTCTGATGGCG |
| | | ACGACGAGGATGGCGGACCTGAGGACCAGGACAGCCCC |
| | | ACCTTCCTGGCtCTGGGCGCCGACGGCAGCCTGGCTGGAT |
| | | TCGTGTCCGTGTCCTACGCCCCATGGAACCGGAGACTGA |
| | | CCATCGAGGACATCGAGGTGGCCCCTGGCCACAGAGGCA |
| | | GAGGAGTGGGCAGGATGCTGATGGCCAGAGCCGAGGAA |
| | | TTCGCCAGAGAGCGGGGCGCTGGCCAGGTGTGGCTGGAA |
| | | GTGACCAACATCAACGCCCCTGCTATCCACGCCTACAGA |
| | | CGCATGGGCTTCAGCCTCTGTGGCCTGGATACCAGCCTGT |
| | | ACGAGTTCACCAGCAGCGCCGGAGAACACGCCCTGTATA |
| | | TGAGCAAGCCTTGCAGCTAA |
| 42 | NATMX resistant | CCTCCTGCTGATGATACCACCTACGAGTTCAGAACCGCC |
| | marker9 | ACCCCTGAGGACACCACACTGGTGGAAGCCCTGGACGGC |
| | | AGCTTCACCACAGCCACAGTGTTCAGAGTGGAAATGGCC |
| | | GAGAACGGCTTTACCCTGAGAGAGACACCTGTGGACCCT |
| | | CCACTGACAAAAGTGTTCCCCGAGGATGAGGGCGACGAG |
| | | GAAGATGACGGCGCTGAAGAGGACGGCGTCAAGGAAGA |
| | | AAACCCCACCTTCCTGGCCGTGGCCCCAGACGGAAGCCT |
| | | CGCCGGCTTCGTGTCCGTGGCTTATGCCAGATGGAACCG |
| | | GCGGCTGACCGTGGAAGACATCGAGGTTGCTCCCGGCCA |
| | | CAGAGGACGGGGCGTGGGCAGAGCCCTGATGAGCAGAG |
| | | CCGAGGAATTCGCCAGAGAGAGGGGCGCCGGACACATCT |
| | | GGCTGGAAGTGACCAACATCAACGCCCCTGCCATCCACG |
| | | CCTACCGCAGAATGGGATTTTCTCTGTGCGGCCTGGACAC |
| | | CAGCCTGTACGAGTTCACAGCCTCTGCCGGCGAGTACGC |
| | | CCTGTACCTGAGCAAGCCTTGTAGAGGCGCTAATAGAGA |
| | | TTAA |
| 43 | NATMX resistant | CCTCCAGCTGATGATACAACCTACGAGATCAGAATCGCC |
| | marker10 | ACACCTGAGGACACCGGCCCCGTGGAAGCtCTGGGCGGC |
| | | AGCTTCACCACAGCCACCGTGTTCAGAGTGGAAATGGCC |
| | | GAGAACGGCTTTACACTGCGGGAAACCCCTGTGGACCCT |
| | | CCACTGACCAAAGTGTTCCCCGAGGATGAGGATGACGAC |
| | | GAGGCCGAAGAGGACGGCGCCAAGGAAGGCCATCCTAC |
| | | CTTCCTGGCCGTGGCTCCCGACGGCTCTCTGGCTGGATTC |
| | | GTGTCCGTGGCCTACGCCAGATGGAATAGAAGGCTGACC |
| | | ATCGAGGACATCGAGGTGGCCCCTGGCCACAGAGGCAGA |
| | | GGCGTGGGCCGCGCTCTGATGAGCAGAGCCGAGGAATTC |
| | | GCCCGGGAAAGAGGAGCCGGCCACATCTGGCTGGAAGTT |
| | | ACAAACATCAACGCCCCTGCTATTCACGCCTATAGACGG |
| | | ATGGGATTTGCCCTCTGTGGCCTGGACACCAGCCTGTACG |
| | | AGTTCACCGCCAGCGCCGGTGAGTACGCCCTGTACCTGA |
| | | GCAAGCCCTGCAGATAA |
| 44 | NATMX resistant | CCACCTGCTGATGATACAACATACGAGATTAGAATCGCC |
| | marker11 | ACACCTGAGGACACCGGCCCCGTCGAGGCtCTGGGCGGC |
| | | AGCTTCACCACAGCCACCGTGTTCAGAGTGGAAATGGCC |
| | | GAAAACGGATTTACCCTGCGGGAAACCCCTGTGGACCCT |
| | | CCTCTGACCAAGGTGTTCCCCGAGGACGAGGACGACGAT |
| | | GAGGCCGAGGAAGATGGCGCCAAGGAAGGCCATCCTAC |
| | | ATTCCTGGCCGTGGCCCCAGACGGCAGCCTGGCCGGATT |
| | | CGTGTCCGTGGCTTATGCCAGATGGAATAGACGGCTGAC |
| | | CATCGAGGACATCGAGGTTGCACCCGGCCACAGAGGAAG |
| | | AGGCGTGGGCCGCGCCCTGATGAGCAGAGCCGAGGAATT |
| | | CGCTAGAGAACGGGGTGCTGGCCACATCTGGCTGGAAGT |
| | | GACCAACATCAACGCCCCCGCTATCCACGCCTACCGGCG |
| | | GATGGGCTTTGCCCTGTGCGGCCTGGACACCAGCCTGTA |
| | | CGAGTTCACCGCCAGCGCCGGCGAGTACGCCCTCTACCT |
| | | GTCTAAACCTTGTAGATAA |
| 45 | NATMX resistant | CCTCCAGCCGACGACACAACATACGAGATCAGAACCGCC |
| | marker12 | ACACCTGAGGACACCGCCCTGGTGGAAGCCCTGGATGGC |
| | | AGCTTCACCACCGCAACAGTTTTCCAGGTGGAAACCGCC |
| | | GAAAACGGCTTTACCCTGCGGGAAACCCCTGTGGACCCC |
| | | CCCCTGACAAAGGTGTTCCCCGAGGATGAGGAATACGAC |
| | | GAGGCCGAGGAAGATGGCGCCAACGAGGGCAACCCTAC |
| | | ATTCCTGGCCGTGACCCCAGATGGCAGCCTGGCTGGCTTT |
| | | GTGTCCGTGGCCTACGCCCGGTGGAATAGACGGCTGACC |
| | | GTCGAGGACATCGAGGTGGCTCCTGGCCACAGAGGAAGA |
| | | GGCGTGGGCAGAGCCCTGATGAGCAGAGCCGAGGAATTC |
| | | GCCAGAGAGAGAGGCGCTGGACACATCTGGCTGGAAGT |
| | | GACCAACATCAACGCCCCTGCTATCCACGCCTATAGAAG |
| | | GATGGGCTTCGCCCTGTGCGGCCTGGACACAACCCTGTA |
| | | CGAGTTCACCGCTTCTGCCGGCGAGTACGCCCTCTACCTG |
| | | AGCAAGCCCTGTCCTTAA |
| 46 | NATMX resistant | ACAACAACCCACGACACAACCTACGCCTTCAGAGTGGCT |
| | marker13 | AGACCTGAGGACGTGGAAGCCATCGCCGCCATCGACGGC |
| | | AGCTTCACAACCGGCACCGTGTTTCAGGTGGCTGTGGCC |
| | | CCTGACGGCTTCACCCTGCGGGAAGTCGCTGTTGACCCCC |
| | | CCCTGGTGAAGGTGTTCCCAGAGGACGACGGCTCTCACG |
| | | ACGCCGAGGGAGAGGATGGCGATAGAAGGACCTACGTG |
| | | GCCGTGGGCGCTGGCGGAGCCGTCGCCGGCTTCACCGCC |
| | | GTGTCCTACACCCCTTGGAACGGCAGACTGACAATCGAG |
| | | GATATCGAGGTGGCCCCTGGCCATAGAGGCAGAGGAATC |
| | | GGCCGGGGACTGATGGAACGGGCCGCTGATTTCGCCCGG |
| | | GAAAGAGGCGCAGGCCACCTGTGGCTGGAAGTGACCAAT |
| | | GTGAACGCCCCTGCCATTCACGCCTATCTGAGACTGGGCT |
| | | TTACATTCTGCGGCCTGGACACCGCCCTCTACCTGGGAAC |
| | | CGAGAGCGAGGGCGAGCAGGCCCTGTATATGAGCATGCC |
| | | CTGTCCTTAA |
| 47 | NATMX resistant | ACCACTCCACACGGCCCGGCCGACGGAATCGTCTACCGC |
| | marker14 | CTCGCCCGCCCCGAGGACGCGGGCGCCATCGAGGCCCTG |
| | | GACAGCTCCTTCACCACCCCCACCGTCTTCGAGGTGACCG |
| | | CCTCCGGCGACGGCTTCGGCTTCCTGCTCCGCGAGGTCCC |
| | | CGTCGACCCGCCCGTGCACAAGGCGTTCCCGCCGGAGGA |
| | | GCACGACGAGCAGGGGTTCGCCGGCGCCCGGGGCCCCGA |
| | | CGTGGACGCGGACGCGCGCACCTTCGTGGCCCTCGACGG |
| | | CGGCGAGCTGTGCGGGTTCGCCGCCGTCGGCTACGCCGC |
| | | GTGGAACCGGCGGCTGACCGTCGAAGACATCGAGGTCGC |
| | | GCCGGGCCACCGGGGCCGCGGGATCGGCAGCGCCCTGAT |
| | | GGAGCGTGCCGCCGAGTTCGCCCGCGAGCGGGGCGCGGA |
| | | GCACCTCTGGCTGGAGGTCAGCTCGGTCAACGCCCCCGC |
| | | CGTGCACGCCTACCGGCGCATGGGATTCACCTTCTGCGG |
| | | CCTCGACACCGCCCTCTACGGCGGCACGCCCTCCGCGGG |
| | | CGAACGGGCGCTGTTCATGAGCCGCCCCTGCCGCTAA |
| 48 | NATMX resistant | ACCACAGTGGACGACACCACCTACGAGTTCAGAACCGCC |
| | marker15 | AGACCTGAAGATGCAGAAGCTGTGGAAGCCCTGGACGG |
| | | ATCTTTCACCACCGCTACAGTGTTCAGAGTGGAAATCGCC |
| | | GAAAACGGCTTTACCCTCAGAGAGACACCTGTGGACAGA |
| | | CCCCTGACCAAGGTGTTCCCAGAGGATGAGAGCGACGGC |
| | | GACGACGACGAGGATGACGGCGGCAGCGAGGACCCTGA |
| | | TTCCCCTACCTTTCTGGCtCTGGGCGCTGATGGCACACTG |
| | | GCTGGCTTCGTTAGCGTGTCCTACGCCCCTTGGAATAGAC |
| | | GGCTGACAATCGAGGACATCGAGGTGGCCCCAGGCCACA |
| | | GAGGAAGAGGCGTGGGCAGGATGCTGATGGCCCGGGCC |
| | | GAGGAATTCGCCCGCGGAAGAGGCGCCGGCCACGTGTGG |
| | | CTGGAAGTGACCAACATCAACGCCCCTGCCATCCACGCC |
| | | TACAGACGGATGGGATTCAGCCTGTGTGGCCTGGACACC |
| | | AGCCTGTACGAGTTCACAAGCAGCGCCGGCGAGTACGCC |
| | | CTGTATATGTCTAAGCCCTGCCCCTAA |
| 49 | NATMX resistant | ACCGCGAACCATGGCACGACGTACGAGTTCCGCACCGCA |
| | marker16 | CGCCCCGAGGACACCGGGGCCATCGAAGCCCTCGACGGG |
| | | TCCTTCACCACCGGCACCGTCTTCGAGGTGGCCGTCACCG |
| | | GCGAGGGGTTCTCCCTGCGCGAGGTCCCGGTGGACCCCC |
| | | CGCTGGTCAAGGTGTTCCCCGAGGACGACGGCAGCGACG |
| | | AGGAGGACGGCGCGGAGGGCGGGGACGGCGACAGCCGC |
| | | ACGTTCGTGGCCGTCTGCGCCGGAGGCGGCCTCGCCGGC |
| | | TTCGCCGCCGTGTCCTACTCGCCGTGGAACCGGCGGCTG |
| | | ACCATCGAGGACATCGAGGTCGCCCCCGACCACCGGGGC |
| | | CGGGGCATCGGCCGTACGCTGATCCGGCACGCCGTGGAC |
| | | TTCGCCCGCGAACGCGGCGCCGGACACCTGTGGTTGGAA |
| | | GTGACCAACGTCAACGCCCCCGCCATCCACGCCTACCGC |
| | | CGCATGGGCTTCGCCTTCTGCGGCCTGGACACCGCCCTGT |
| | | ACCAGGGCACCGAGTCCGAGGGCGAGCACGCGCTCTACA |
| | | TGAGCATGCCCTGCCCCTAA |
| 50 | NATMX resistant | ACAACCGCCCATGGCCCTGCCGACGGCATCGTGTACCGG |
| | marker17 | CTGGCCAGGCCTGAAGATGCCGGCGCCGTCGCCGCTCTG |
| | | GACAGCAGCTTCACAACAAGAACCGTTTTCGAGGTGGCA |
| | | GTGAGCGGCGACGGCAGCGGATTTCTGCTGCGCGAGGTG |
| | | CCCGTGGACCCCCCAGTGCGGAGAGCCTTCCCTCCTGAG |
| | | GAACACGACGAGCAGGGCATCGCCGGCCCAAGAGGAGC |
| | | TGATGTGGACGCCGATACCAGAACCTTCGTGGCTCTGGA |
| | | TTCTGGAGAGCTGTGCGGCTTCGCCGCCGTGGGCTACGC |
| | | CGCCTGGAACCGGCGGCTGACAGTGGAAGACATCGAGGT |
| | | CGCTCCTGGCCACAGAGGAAGAGGCATCGGAAGCGCCCT |
| | | GATGGGTTGTGCCGCTGAATTCGGCAGAGAGCGGGGCGC |
| | | CGAGCACATCTGGCTGGAAGTGTCCAGCGTGAACGCCCC |
| | | TGCCGTGCACGCCTATAGAAGAATGGGCTTTACCTTCTGC |
| | | GGCCTGGACACCGCCCTCTACGGCGGCACCCCTGCCGCT |
| | | GGCGAGCAGGCCCTGTTCATGTCTAGACCCTGCAGATAA |
| 51 | NATMX resistant | ACCACCGCACCCGGCTCCGCCGACGGCATCGTCTACCGC |
| | marker18 | CCGGCCCGCCCCGAGGACGCCGGCGCCATCGAGGCCCTG |
| | | GACAGCTCCTTCACCACCGCCACCGTCTTCGAGGTGACC |
| | | GTCCACGCCACGGGCTTCACCGTGCGCGAGGTCCCGGTG |
| | | GACCCGCCCCTGCGCAAGGTGTTCCCGCCCGAGGAGCAC |
| | | GACGAGCAGGCGCTCGGCGGCGGCGCCCCGGACTCGGAC |
| | | GGCGACGCGCGCACGTACGTGGCCCTCGACGGCGGCCGG |
| | | GTCTGCGGCTTCGCCGCCGTCGGCTACACCCCCTGGAACC |
| | | GCCGGCTGACCGTCGAGGACATCGAGGTCGCGCCCGGCC |
| | | ACCGCGGGCGCGGCATCGGCCGCGCGCTGATGGAGCACG |
| | | CCGCCGACTTCGCCCGCGAGCGCGGCGCCCGGCACCTGT |
| | | GGCTGGAGGTCAGCACGGTCAACGCCCCGGCCGTGCACG |
| | | CCTACCGGCGGATGGGGTTCACCCTCTGCGGGCTCGACA |
| | | CCACGCTGTACGACGCCACCCCGGCCGCGGGGGAGCGCG |
| | | CGCTGTACATGAGCCGGCCCTGCGGCTAA |
| 52 | NATMX resistant | ACCACCCCACACGGCCCGGGCGGCGCAGTCGTCTACCGC |
| | marker 19 | CTCGCCCGCCCCGAGGACGCCGGCGCCATCGAGGCCCTG |
| | | GACAGCTCCTTCACCACCCCCACCGTCTTCGAGGTGGAC |
| | | GCCTCCGGCGACGGCTGGGGGTTCCTGCTCCGGGAAGTC |
| | | CCCGTCGACCCGCCCCTGTACAAGGTGTTCCCGCCCGAG |
| | | GAGCACGGCGAGCAGGGGTACGCCGGCGCCCGGGGGCC |
| | | CGACGTGGACGCGGACACGCGCACCTTCGTGGCCCTCGA |
| | | CGGCGGCGAGCTGTGCGGGTTCGCCGCCCTCGGCTACGC |
| | | CGCCTGGAACCGGCGGCTGACCATCGAGGACATCGCGGT |
| | | CGCGCCCGGCCACCGGGGCCGGGGGATCGGCAGCGCCCT |
| | | GATGGAGCGTGCCGCCGACTTCGCCCGTGAACGGGGCGC |
| | | GGAACACCTCTGGTTGGAGGTCAGCTCGGTCAACGCCCC |
| | | CGCCGTGCACGCCTACCGGCGCATGGGATTCACCCTGTG |
| | | CGGCCTCGACACGGACCTCTACGGCGGCACGCCCTCGGC |
| | | GGGCGAGCGGGCCCTGTTCATGAGCCGCCCCTGCCGCTA |
| | | A |
| 53 | NATMX resistant | CCTAGCGCCGACGACACCACCTACGAGATCAGAACAGCC |
| | marker20 | ACCCCTGAGGACGCCGGACTCGTGGAAGCCCTGGACGGC |
| | | AGCTTCACCACAGCAACCATTTTCCAGGTGGAAACCGCT |
| | | GAGAATGGATTTACCCTGCGGGAAACCGCTGTGGACCCT |
| | | CCCCTGACCAAGGTGTTCCCCGACGAGGAAGATGAGAAC |
| | | GTTAGCGCCGATGAAGGCGACCAGGAACCTCAGGGCGCT |
| | | CCTACCTTCCTGGCTCTGGCCCCAGATGGCAGCCTGGCCG |
| | | GCTTCGTGTCCGTGGCCTACGAGAGATGGAACAGACGGC |
| | | TGACAATCGAGGACATCGAGGTGGCCCCTGGCCACCGGG |
| | | GACGCGGCGTGGGCAGAGCCCTGATGAGCAGAGCCGAG |
| | | GAATTCGCCAGAGAGCGGGGAGCCGGCCACATCTGGCTG |
| | | GAAGTGACAAACATCAACGCCCCTGCCATCCACGCCTAT |
| | | AGAAGAATGGGCTTTGCCCTGTGCGGCCTGGATACATCT |
| | | CTGTACGAGTTCACAGCTTCTGCCGGCGAGTACGCCCTGT |
| | | ACCTGAGCAAGCCATGTAGAGGCGCCAACCGGGACTAA |
| 54 | Blasticidin marker 1 | CCACTGACCCCTGAAGAGACAGCCCTGGTGGACGCCGCC |
| | | ACATCTATCATCACCAGCATCCCCATCAGCGACACCTAC |
| | | AGCGTGGCCAGCGCCGCTAGATCCACAGACGGCAGAATC |
| | | TTCACAGGCGTGAACGTGTTCCACTTCACCGGCGGACCTT |
| | | GTGCCGAGCTGGTTGTGCTCGGCTCTGCCGCTGCTGCCGG |
| | | CGCCACCCAGCTGACCCACATCGTGGCCGTGGCTAATGA |
| | | GAACCGGGGAATCCTGAGCCCCTGCGGCCGGTGCAGACA |
| | | GACCCTGATCGACCTGCAGCCTGGCATTAAGGTGATCGT |
| | | GCTGGATAGAGGCGAGCCTAGAGGAGTGCGGGTGGAAG |
| | | AACTGCTGCCTTTTGCCTACTTCGCCGATTAA |
| 55 | Blasticidin marker 2 | ACCACCCTGAGCTTCGTGGCCGCCACCGAGCTCGCCGCT |
| | | ACACTGGGAGATGACCCTAACCACACCGTGGCCGCCGCT |
| | | GCtCTGGGCCTGAACGGCAATATCTACGCCGGCGTGAACA |
| | | ACCACCATTTCAACGGCGGACCTTGCGCCGAACTGGTCG |
| | | TGCTGGGCGTTGCAGCTAGAGCCCACGCCGGAAATCTGG |
| | | CCACAATGGTGGCCGTGGGCGACGGCGGCAGAGGCGTG |
| | | ATCTCTCCATGTGGCCGGTGCAGACAGGTGATGCTGGAT |
| | | CAGCACCCCGACATCTGCGTGCTGGTGCCTCTGGACGAC |
| | | TAA |
| 56 | Blasticidin marker 3 | CCATTCGAGCCTCTGTCCGCCACAGGCCAGAACCTGATC |
| | | GACACCGCCACCACCGTGATCAACAACATCCCCGTGTCC |
| | | GATTTCTACAGCGTGGCCAGCACAGCCATCTCTGATGAC |
| | | GGCAGAGTGTTCAGCGGCGTGAACGTGTACCACTTCACC |
| | | GGCGGACCTTGTGCCGAGCTGGTGACtCTGGGCGTTGCAG |
| | | CCGCCGCTGGTGCTCAGAAGCTGACCCACATCGTGGCCG |
| | | TGGCTAATCAAAATAGAGGCATCCTGAGCCCTTGCGGCC |
| | | GGTGCAGACAGGTGCTGACCGACCTGCACCCTGGAATCA |
| | | AGGTGGTCGTCGTGGGCAAAGAGGGCGCCCTGATGCTGT |
| | | GGCCTAGGCTGTACGCCCAGTGGAAGCACGCCGGCAAGC |
| | | CCCCCTACCTGATTGGATCTAGCCACTTTGGCTGCCAGGG |
| | | CAACAGCGTGTATAAGCACACCCATGTGAAACTGACAAC |
| | | AAGCCTGCTGAGCCTGAGAGTGAAGGCCACCCTGACACC |
| | | TCGGACCAAGGAACTCTACTGCGAGGAATAA |
| 57 | Blasticidin marker 4 | CCATTCGAGCCTCTGTCCGCCACAGGCCAGAACCTGATC |
| | | GACACCGCCACCACCGTGATCAACAACATCCCCGTGTCC |
| | | GATTTCTACAGCGTGGCCAGCACAGCCATCTCTGATGAC |
| | | GGCAGAGTGTTCAGCGGCGTGAACGTGTACCACTTCACC |
| | | GGCGGACCTTGTGCCGAGCTGGTGACtCTGGGCGTTGCAG |
| | | CCGCCGCTGGTGCTCAGAAGCTGACCCACATCGTGGCCG |
| | | TGGCTAATCAAAATAGAGGCATCCTGAGCCCTTGCGGCC |
| | | GGTGCAGACAGGTGCTGACCGACCTGCACCCTGGAATCA |
| | | AGGTGGTCGTCGTGGGCAAAGAGGGCGCCCTGATGCTGT |
| | | GGCCTAGGCTGTACGCCCAGTGGAAGCACGCCGGCAAGC |
| | | CCCCCTACCTGATTGGATCTAGCCACTTTGGCTGCCAGGG |
| | | CAACAGCGTGTATAAGCACACCCATGTGAAACTGACAAC |
| | | AAGCCTGCTGAGCCTGAGAGTGAAGGCCACCCTGACACC |
| | | TCGGACCAAGGAACTCTACTGCGAGGAATAA |
| 58 | Blasticidin marker 5 | GCAACCATCTACAGCCATCTGTCTGAGGCCGAACAAAAT |
| | | CTGATCGAGGTGGCCGCTAAAACAATCGAGGCCATCCCC |
| | | GTGTCCGAGGATTATAGCGTTGGATCTGCCGCCCTGGCC |
| | | GAGGACGGCAGAATCTTCACCGGCATCAACGTGTACCAC |
| | | TTCACCGGCGGCCCTTGTGCCGAGCTGGTGGTGCTGGGA |
| | | GTGGCTGCTATGGCCGGACCTCCAAAGCTGACCCACATC |
| | | GTGGCCGTCGGCAACCAGGGCAGAATGATCCTGAGCCCT |
| | | TGCGGCCGGTGCAGACAGGTGCTCGGCGACCTGCACCCC |
| | | GACATCAAGGCCATTGTGCGGGACGCCGATGGCAGCGTG |
| | | AAGGTGGAAAAGGTCCAGGACCTGCTGCCTGCCAGATAC |
| | | GTGATCCCTGATGCCACAGTGGAAAGCATGTAA |
| 59 | Blasticidin marker 6 | TCTAGCGCCGCAGATCAGGCCCTGATCGAGCGGGCCAGA |
| | | GCCCTGATTGAGTCCCTGCCCGACGACGAGAACCACACA |
| | | GTGGCTGCTGTGGCCCTGGACACCGCCGGCCGCCACTTT |
| | | GACGGCGTTAATCTGTATCACTTCACCGGCGGACTGTGC |
| | | GCCGAGCCTGTGGTCCTCGCCGTGGCCGCCGCCCAGCAG |
| | | GCCGCTCCTCTGGAAGTGGTGGTGGCCGTGGGCAACCGG |
| | | GGCAGAGGCGTGCTGGCCCCATGTGGCCGGTGCAGACAG |
| | | ATCCTGTTCGACTACCATCCTGATATCCAGGTGCTGGTGC |
| | | CCCACGGACCTCAGATCAGAAGAGTGGGCATCCGGGAAC |
| | | TGCTGCCTTACACCTACAACTGGCACGCCCAAACAGATA |
| | | GAGAGCACGGCGAGGCCAGCAGACAGGCTGAATAA |
| 60 | Blasticidin marker 7 | GCCACCATCTACAGCCACCTGAGCAAGGCCGAGCAGAAC |
| | | CTGATCGAGGTGGCCACAAAGACCATCGAGGCCATTCCT |
| | | GTGTCCGAGGACTATTCTGTCGGAAGCGCCGCCCTGGCC |
| | | GAGGACGGCAGAATCTTCACCGGCATCAATGTGTACCAC |
| | | TTCACCGGCGGTCCTTGCGCCGAACTGGTGGTGCTGGGC |
| | | GTGGCCGCTATGGCCGGCCCCCCCAAGCTGACACACATC |
| | | GTGGCTGTTGGCAACCAGGGCCGCATGATCCTGTCTCCTT |
| | | GTGGCAGATGCAGACAAGTGCTCGGAGATCTGCATCCAG |
| | | ACATCAAGGCCATCGTGCGGGTCGCTGATGGCAGCGTGC |
| | | GGGTGGAAAAAGTGCAGGACCTGCTGCCTGCCAGATACG |
| | | TGATCCCTGACACCACAGTGGAAAGCATCTAA |
| 61 | Blasticidin marker 8 | GACCTGACACCTGAGGAAATCAAGCTCGTGGAAGTGGCC |
| | | AAGGCCACCATCCAGTCCATTTCTACAAGCGACACCTAC |
| | | AGCGTGGCTTCTGCCGCCCTGAGCGCCGACGGAAGAACA |
| | | TTCAGCGGCGTGAACGTGTTCCACTTTACCGGCGGACCTT |
| | | GTGCCGAGCTGGTGGTGCTGGGCAGCGCCGCTGGCGCTA |
| | | ACGCCCAGAAACTGAAGACCATCGTGGCCGTGGGAGATG |
| | | ACGGCGAGAAGGGCGTGGTGCTGAACCCCTGCGGCCGGT |
| | | GCAGACAGGTGCTGAGAGATCTGCAACCTAGCATCAATG |
| | | TGGTCGTCGTTAAGGGCGGCAAGCTGAAAAGCATCTCCA |
| | | TCAACGAGCTGCTGCCATACGCCTACGACACCCGGGAAT |
| | | AA |
| 62 | Blasticidin marker 9 | GCCGATCTGCGGGACCTGTCTGATGCCGACTTCGCCCTGA |
| | | TCGAGCACGCCAGACAGATCGTGGAAAGCAACGGCGAC |
| | | GGCTCTATCAGCACCATGGGCAGCGCCGCTAGGTCCACC |
| | | ACCGGCGAGATCTTTGGCGCCATTAACCTGTACCATTTCA |
| | | CCGGAGGACCTTGTGCCGAGCTGGTGGTCCTGGGCGTGG |
| | | CTGCCGCCCACGGCGTGCGGAGCCTGGAAACAATCGTGG |
| | | CCGTGGGTGACGAGGGCAGAGGCCCTGTGGGACCTTGCG |
| | | GCCGGTGCAGACAAGTGCTGTTCGACTACCACCCCCAGA |
| | | TCAGAGTGCTCCTGCCTACCGGCGCTGAGGGAGTTAAGA |
| | | GCGTGGCCATCGGCGATCTGCTGCCATACGGCGGCAGAT |
| | | GGGACGTGGAACTGGGAACACAGCCTTATGAGCCTACAT |
| | | AA |
| 63 | Blasticidin marker 10 | GGACTGAACGCCAAGGAAACCAAGCTGGTTGACATCGCC |
| | | AGAGATACCATCAACGCCATCCCCCGGAGCTCTACACAC |
| | | AGCGTGTCCAGCGCCGCTCTGAGCATCAGCGGCCAGGTC |
| | | TTTACCGCCGTGAACGTGTTCCATTTCACCGGCGGCCCTT |
| | | GCGCCGAGCTGGTGGTGCTCGGCGTGGCTGCTGGCGCCG |
| | | GAACACCTCGGCTGTCTCACATCGTGGCCGTGGGCGAAG |
| | | ATGGCCACGACGGCATCATCCTGAATCCTTGTGGCAGAT |
| | | GCAGACAGGTGCTGTACGACCTGCACCCCGGCATTAGAG |
| | | TGATCGTGCAGAAAGGCGGAAAGGCCGAGAGCGTGCTG |
| | | ATCGACGAGCTGCTGCCATACGCCTACGAGCCTCGCGAA |
| | | TAA |
| 64 | Blasticidin marker 11 | GATCTGACACCTGAGGAAACCAACCTGATCGAGATCGCC |
| | | AGAACCACCATCAATGCCATCCCCAAGTCTGATACCTAC |
| | | AGCGTGGCCAGCGCCGCTCTGAGCGTGGACGGCAGAATC |
| | | TTCACCGGCGTGAACGTGTACCACTTCACAGGAGGACCT |
| | | TGCGCCGAACTGGTGGTGCTGGGCGTTGCTGCTGGCGCC |
| | | GGAACACCAAGACTGAGCCACATCGTGGCCATCGGCGAA |
| | | GATGGCCAGGACGGCGTGGTCCTCAACCCCTGCGGCAGG |
| | | TGTAGACAGGTGCTGCACGACCTGCATCCTGGCATCAGA |
| | | GCCATTGTGCGGAAGGACGGCGAGGCCAAATGCGTGTCC |
| | | ATCAACGAGCTGCTGCCTTGGGGCTACGGCCCTCGGGAC |
| | | TAA |
| 65 | Blasticidin marker 12 | CCCCTCCACGACTCCGAGGTCCGGCTGATCGACGCGGCC |
| | | GAGGCGCTCGCCCGGACGCTCGGCGCGGACCCGGACCAC |
| | | ACCATGGCGGCCGCGGCCCTCGACGCCGCCGGCCGGATC |
| | | CACGTCGGCGTCAACGTCCTGCACTTCACGGGCGGCCCG |
| | | TGCGCGGAGCTCGTCGCGCTGGGTGCCGCGGCCGCCGCG |
| | | AATGCGGGACCGCTCGTGGCGATGGCGGCGGTGGGCGAC |
| | | GGCGGCCGGGGGATCGCCCCGCCCTGCGGCCGGTGCCGC |
| | | CAGGTGATGCTGGATCTCCAGCCCGGCATCCGCGTGGCC |
| | | GTGCCCGGCGCCGACGGGCCCGAGATCGTCGCGATCCGC |
| | | GACCTGCTGCCGGTCTCGTACGCCCGACCCGACGCGTAA |
| 66 | Blasticidin marker 13 | ACGCGACGCCACGAGCCCGGTCCTCGGCCCGAGCCCGGC |
| | | TCCGATCCTGGCCCTGAGCCCGAGCCCGGCCCCGGACCC |
| | | GAGCCCGGACCCGGACCCGAGCCCGGACCCGAGCCCGGC |
| | | CCCCGCCCCACCCCTCAGCCCGACCCCGGTCCCGACCCC |
| | | GCCCTCGTGCGGGCCGCCGCCGCGCTCGCCGCGCGGCTC |
| | | GGGGCGGACGACAACCACTCGGTGGCCGCGGCGGCCCG |
| | | GGACGCCGGGGGCCGGGTGGTCACCGGTGTGAACGTGTA |
| | | CCACTTCACCGGCGGCCCCTGTGCCGAACTCGTCGTGCTG |
| | | GGCGCCGCCGTGGCCGAGGGCGCGGGACCGCTCGTGCGG |
| | | ATCGTGGCGGTGGGTGACCGGGGGCGCGGGGTGATGCCG |
| | | CCGTGCGGTCGATGCCGACAGGCGCTGCTCGACCTGTGG |
| | | CCGGGTATCGAGGTGCTGGTGCCGGGCGCCGAGGGCGGG |
| | | GTGCGTGGCGTGCCCGTGCGCGAACTGCTCCCTCATACAT |
| | | ACGTGTAA |
| 67 | Blasticidin marker 14 | ACCACCCTAACCCCCCAAGAAGCCTCCCTCCTCGAAACC |
| | | GCCACAAAAACAATAACCAGCATCAAACCCTCCAACACG |
| | | CACAGCGTCGCCAGCGCCGTCCTCGCCTCCGACGGCCGC |
| | | GTCTTCTCCGCCGTAAACGTCTACCACTTTACCGGCGGCC |
| | | CTTGTGCCGAACTCGTCGCCCTcGGGAATGCTGCCGCGGC |
| | | CGGGGCCGAGGAGCTCACCCATATCGTGGCCGTCGAGGA |
| | | TACCCGGCGTATCTTGAGTCCCTGTGGACGGTGTCGGCA |
| | | GGTTTTGTTGGACTTGTGGCCTGGCATTAGGGTTATTGTT |
| | | TTGGGGGAAGAGGGGCCtAGGGTTGTTGGCATTGCGGAG |
| | | TTGTTGCCTTTTGCTTATTCGTGGCCTGGGGAGGAGTAA |
| 68 | Blasticidin marker 15 | CCACTGCACGACAGCGAGGCCAGACTGATCGACGCCGCT |
| | | GAAGCCCTGGCCCGGACCCTcGGCGCTGATCCTGATCACA |
| | | CAATGGCCGCCGCTGCCCTGGATGCCGGAGGCAGAATCC |
| | | ACGTGGGCGTGGACGTGCTGCATTTCACCGGCGGCCCTT |
| | | GTGCCGAGCTGGTTGCCCTCGGCGCCGCCGCTGCAGAAA |
| | | ACGCCGGCCCCCTGGTGGCCATGGCCGCTGTGGGAGATG |
| | | GCGGAAGAGGCATCGTGCCCCCCTGCGGCCGGTGCAGAC |
| | | AGGTGATGCTGGACCTGCAGCCTGGCATCCGGGTGGCCG |
| | | TGTCTGGCGCCGACGGCCCTGAGATGGTCGGAATCGGCG |
| | | ACCTGCTGCCTGTGAGCTACGCCAGACCTGACGAGTAA |
| 69 | Blasticidin marker 16 | CCCCTCACATCTTCTGAAACCAACCTCGTAAACCTAGCCA |
| | | TCAAGGCAATAACCCAAATCCCCAAATCAGAAGACTACA |
| | | GTGTCTCCAGCGCCGCTCTCTCAGAAGACGGCCAGATCTT |
| | | CACCGGAATAAATGTCTACCACTTCACCGGCGGCCCCTG |
| | | TGCGGAACTCGTCACACTGGGCGTCGCTGCGCTCGCCGG |
| | | ACCCCCGAAACTCACTCTTATCGTCGCTGTTAGCAATGAT |
| | | GGCAGGATCCTCAGCCCCTGTGGAAGATGCAGACAGGTG |
| | | CTAAGGGATTTGCATCCGGGTATTAAAGTTATTGTTCCTA |
| | | AGGAAGGGGGCCCGGAAGTGGTGGGGATTGATGATTTGT |
| | | TGTAA |
| 70 | Blasticidin marker 17 | TTAGATTATAAGGATATGGAACTTATTGAAAAGGCAAGT |
| | | GAAATATTAAAGAAAAATTATGATAGAGAAAATTACAAT |
| | | CATACAGTTGCAGCAGCAGTTAAATGTAGTAGTGGTAAT |
| | | ATATATTTGGGGATTAACGTATTTTCTCTACATGGGGCAT |
| | | GTGCTGAACAAGTGGCAATAGGTACTGCTGTTACAAACG |
| | | GAGAAAAGGATTTTAAATGTATTGTTGCAATAAGAGGTG |
| | | AGAATGGGGATGAAGTATTATCACCATGTGGAAATTGTA |
| | | GACAAATGTTATCAGACTATTGTCCTAACTGTGAAGTGAT |
| | | TATACAAACAAATGATGGATTGCAAAAGGTATTAGCTAA |
| | | AGATTTAATACCTTTTGCATATAAATCTGAAAGTTAA |
| 71 | Blasticidin marker 18 | ACCACCGGGATCCACCCCGTCGACCACGAACTCGTCCGT |
| | | GCCGCGACCGACGTCGCGCGCACCCGGTGCCGGGGCGAC |
| | | AACCACACCATGGCGGCAGCGGCCCGTGCCCGGGACGGC |
| | | CGCGTCATCACGGCCGTGAACGCCTACCACTTCACCGGA |
| | | GGCCCGTGCGCCGAACTGGTTCTCATCGGCACGGCcGCCG |
| | | CgCAGGGAGCCTACGAACTGGACACCGTCGTCGCCGTGG |
| | | GCGACCGCGACAGGGGAGTGGTGCCGCCCTGCGGCCGCT |
| | | GCCGCCAGGTCCTGCTCGACTACTTCCCCTCCCTCCGGGT |
| | | CATCGTCGGGTCCGGCGACCGCCTCCGCGCCGTCCCCGT |
| | | GACGGATCTGCTGCCGGACAGCTACGTCTGGGCCGACCA |
| | | CCAGCCGGACACCGACTAA |
| 72 | Blasticidin marker 19 | GATGCTGCGGAAACCCTGGCGCGAAGCCTCGGCGACAAC |
| | | GACAATCACACCGTTGCAGCAGCGGCGATGGACGTTGAT |
| | | GGACGCATTCACCAAGCAGCGAATGTCTACCACTTCACC |
| | | GGTGGTCCGTGCGCCGAACTCGTTGCCTTAGGAGTTGCG |
| | | GCCGCTGCGGGAGCAAAGCAGCTTTTGACTATTGCCGCT |
| | | GCTGGTGACCGAGGGCGGGGTTTGATTCCTCCATGTGGT |
| | | CGATGCCGACAAGTTCTCCTCGATCATCATCCGGATATTC |
| | | TTGTCGCGGTCCCTGCGGAGAAGGGCCCTGTCGTTCGGC |
| | | CCGTCCGGAAGCTCCTGCCAGTAGTGCCCCGTAGAGTGG |
| | | TGTAA |
| 73 | Blasticidin marker 20 | GACGTGGACGGCAGAATCCACCAGGCCGTGAACGTGTAT |
| | | CACTTCACCGGCGGACCTTGTGCCGAGCTGGTGGCCCTcG |
| | | GCGTTGCTGCCGCCGCTGGCGCCAAGCAGCTGCTCACCA |
| | | TCGCCGCCGCTGGAGATAGAGGAAGAGGCCTGATCCCTC |
| | | CTTGCGGCCGGTGCAGACAGGTGCTGCTGGAACACCACC |
| | | CCGACATCCTGGTCGCCGTGCCAGCCGAGAAAGGCCCTG |
| | | CCGTGCGCCCCGTGCGGAAACTGCTGCTGGACACCTACT |
| | | TCTACCCTGACGCCCAAGGCAGGAGAATCTTTAGATTCA |
| | | ACAAGCGGTACCACGACGCCGTGATCTCTGGCGAAAAGA |
| | | CAACCACAATTAGATGGGACGAGAGCGTCGAGGCCGGCC |
| | | CCGCTACATTTGTGTTCGAGGACCACCCTGAGTTCGCCCA |
| | | TGTGGAAGGCGAGATCATCAGCGTGGGTCAGACCAGACT |
| | | GCAGGACCTGGATGCCGAACGGGCCAGAGGCCTGAAGG |
| | | CCCACTACCCCAGCATGCCTGATGATGCTGAACTGAGCA |
| | | GAGTGTCCTTCCGGGTGCACGGCGTGCGGTAA |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A system for modifying a plurality of target nucleic acids comprising:
a donor nucleic acid comprising a cargo sequence encoding one or more selectable markers, wherein one or more nucleic acid sequences encoding the one or more selectable markers are optionally: adjacent, individually or as a group, to one or more nucleic acid sequences encoding an internal ribosome entry site (IRES) or a ribosome skipping peptide;
and/or operably linked to a promoter;
a first guide RNA complementary to at least a portion of the donor nucleic acid or a nucleic acid encoding the first guide RNA;
a plurality of second guide RNAs each of which is complementary to at least a portion of one of the plurality of target nucleic acids, or one or more nucleic acids encoding the plurality of second guide RNAs;
a first RNA-guided endonuclease, or a nucleic acid encoding thereof, configured to bind to the first guide RNA; and
a second RNA-guided endonuclease, or a nucleic acid encoding thereof, configured to bind to the plurality of second guide RNAs.

2. The system of claim 1, wherein the donor nucleic acid further encodes an insert.

3. The system of claim 2, wherein the insert is a tag, a binding protein or domain thereof, an effector protein or domain thereof, a localization signal, a regulatory element, or a combination thereof.

4. The system of claim 1, wherein the cargo sequence encodes two or more selectable markers.

5. The system of claim 1, wherein one or more nucleic acid sequences encoding the one or more selectable markers are each individually adjacent to one or more nucleic acid sequences encoding an internal ribosome entry site (IRES) or a ribosome skipping peptide.

6. The system of claim 1, wherein the cargo sequence further encodes a transcription factor configured to activate the promoter operably linked to the one or more selectable markers.

7. The system of claim 1, wherein the plurality of second guide RNAs are in a plurality of cells, wherein each cell expresses a single second guide RNA complementary to at least a portion of one of the plurality of target nucleic acids.

8. The system of claim 1, wherein the first and/or second RNA-guided endonuclease is a Cas nuclease.

9. The system of claim 8, wherein the first RNA-guided endonuclease and second RNA-guided endonuclease are orthogonal Cas nucleases.

10. The system of claim 8, wherein the Cas nuclease is Cas9.

11. The system of claim 1, wherein the first and second RNA-guided endonucleases are encoded on a single nucleic acid.

12. A method for modifying one or more or all of a plurality of target nucleic acids comprising contacting a plurality of target nucleic acids with:
a donor nucleic acid comprising a cargo sequence encoding one or more selectable markers and optionally an insert,
wherein one or more nucleic acid sequences encoding the one or more selectable markers are optionally: adjacent, individually or as a group, to one or more nucleic acid sequences encoding an internal ribosome entry site (IRES) or a ribosome skipping peptide; and/or operably linked to a promoter, and
wherein the insert is a tag, a binding protein or domain thereof, an effector protein or domain thereof, a localization signal, a regulatory element, or a combination thereof;
a first guide RNA complementary to at least a portion of the donor nucleic acid or a nucleic acid encoding the first guide RNA;
a plurality of second guide RNAs each of which is complementary to at least a portion of one of the plurality of target nucleic acids, or one or more nucleic acids encoding the plurality of second guide RNAs;
a first RNA-guided endonuclease, or a nucleic acid encoding thereof, configured to bind to the first guide RNA; and
a second RNA-guided endonuclease, or a nucleic acid encoding thereof, configured to bind to the plurality of second guide RNAs.

13. The method of claim 12, wherein the plurality of target nucleic acids are within a cell or cell population and contacting a plurality of target nucleic acids comprises introducing into the cell or cell population.

14. The method of claim 12, wherein one or more or all of the plurality of target nucleic acids encodes a gene or gene product.

15. The method of claim 14, wherein each cell in the cell population comprises a single second guide RNA.
